SEQUENCE LISTING (2 sequences)

SEQ ID NO: 1 - DNA, 6051 bp, Homosapiens
  SCN5A
atggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcgtatgtatcagaaaatataaaactaggcaatttgtcggctcttcgaactttcagagtcctgagagctctaaaaactatttcagttatcccagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtga

SEQ ID NO: 2 - protein, 2016 aa, Homosapiens
  hNav1.5
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQQESQPVSGGPEAPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

(19) United States
(12) Patent Application Publication
(10) Pub. No.: US 2018/0068053 A1
(43) Pub. Date: Mar. 8, 2018

(54) SYSTEMS AND METHODS OF SELECTING COMPOUNDS WITH REDUCED RISK OF CARDIOTOXICITY USING CARDIAC SODIUM ION CHANNEL MODELS

(71) Applicant: THE GOVERNORS OF THE UNIVERSITY OF ALBERTA, Edmonton (CA)

(72) Inventors: Khaled BARAKAT, Edmonton (CA); Marawan AHMED, Edmonton (CA); Aravindhan GANESAN, Edmonton (CA); Michael HOUGHTON, Edmonton (CA); Lorne TYRRELL, Edmonton (CA)

(21) Appl. No.: 15/669,843

(22) Filed: Aug. 4, 2017

Related U.S. Application Data
(60) Provisional application No. 62/371,675, filed on Aug. 5, 2016.

(51) Int. Cl. G06F 19/12; G06F 19/24; G06F 19/16; G01N 33/50; G01N 33/68; G06F 19/00

(52) U.S. Cl. CPC G06F 19/12; G06F 19/24; G06F 19/16; G01N 2333/705; G01N 33/5061; G01N 33/6872; G06F 19/701; G01N 33/5014

(57) ABSTRACT

Provided herein are systems and methods for selecting compounds that have reduced risk of cardiotoxicity or which are not likely to be cardiotoxic. As an example, a system and method can include a computational dynamic model combined with a high throughput screening in silico that mimics an important ion channels associated with cardiotoxicity, namely the human cardiac sodium ion (hNaᵥ1.5) channel). In certain embodiments, steered molecular dynamics simulations are used to identify key residues of the channel's permeation pathways that are then used in the high throughput screening. Also provided herein are systems and methods for redesigning compounds that are predicted to be cardiotoxic based on the model and the high throughput screening.

This application claims the benefit of and priority to U.S. Provisional Application No. 62/371,675, filed Aug. 5, 2016, the entire content of which is incorporated by reference herein in its entirety.

### 1. TECHNICAL FIELD

This application relates generally to compounds and cardiotoxicity and more generally to processor-implemented systems and methods for analyzing compounds with respect to cardiotoxicity using models of the human cardiac sodium ion channel.

### 2. BACKGROUND

Cardiotoxicity is a leading cause of attrition in clinical studies and post-marketing withdrawal. Recent investigations suggest that the human cardiac sodium ion channel (also referred to as the hNaᵥ1.5 channel) is implicated in cardiotoxicity, and screening of candidate drugs for activity against this cardiac ion channels is therefore recommended to minimize any potential cardiac adverse effects.

In particular, the hNaᵥ1.5 channel is shown to be responsible for initiating the myocardial action potential (Roden et al., 2002, “Cardiac ion channels,” Annu. Rev. Physiol., 64, 431-475). Thus, mutations in hNaᵥ1.5 have been associated with a wide range of cardiac diseases, such as long QT syndrome 3 (LQT3), Brugada syndrome 1 (BRGDA1) and sudden infant death syndrome (SIDS) (Campuzano et al., 2010, “Genetics and cardiac channelopathies,” Genet. Med., 12, 260-267; Remme, 2013, “Cardiac sodium channelopathy associated with SCN5A mutations: electrophysiological, molecular and genetic aspects,” J. Physiol., 591, 4099-4116).

Generally, the hNaᵥ1.5 channel is part of a larger family of voltage gated sodium ion channels (VGSCs) that are presumed to be one of the most important macromolecules in the human body. VGSCs regulate several physiological functions and disorders related to these channels have been implicated in many diseases (Yu et al., 2003, “Overview of the voltage-gated sodium channel family,” Genome Biol., 4, 207). VGSCs are responsible for initiating the action potential in all excitable cells and many isoforms of VGSCs are spread all over the body organs. VGSCs are usually present with high propensities on certain organs, forming tissue-specific distributions. The hNaᵥ1.5 channel represents the major VGSC isoform on the human heart (Roden et al., 2002, “Cardiac ion channels,” Annu. Rev. Physiol., 64, 431-475). Architecturally, all eukaryotic VGSCs have been shown to possess a similar overall folding pattern. A functioning unit of a normal VGSC is called the α-subunit and is usually expressed together with one or more auxiliary subunits defined in the literature as the β-subunits.

The α-subunit (the pore-forming subunit) is capable of forming a fully functional channel and can generate electrical pulses independent from the β-subunits. Topologically similar to other VGSCs, the α-subunit of hNaᵥ1.5 is a 220-kDa protein is organized into four domains (DI-DIV) and is covalently or non-covalently attached to the β-subunits through the N and C-termini (Brackenbury et al., 2011, “Na Channel beta Subunits: Overachievers of the Ion Channel Family,” Front. Pharmacol., 2, 53).

The β-subunits are responsible for hNaᵥ1.5 localization and interaction with cell adhesion biomolecules as well as the intracellular and extracellular matrix (Catterall et al., 2005, “International Union of Pharmacology. XLVII. Nomenclature and structure-function relationships of voltage-gated sodium channels,” Pharmacol. Rev., 57, 397-409). Incorporating a β-subunit within the hNaᵥ1.5 channel can modify the channel's gating kinetics and voltage dependence.

Molecular modeling techniques have provided some guidance in screening drug candidates for their blocking ability to block cardiac channel proteins (PCT Publ. No. WO2015-085432; Stansfeld et al., 2007, “Drug Block of the hERG Potassium Channel: Insight from Modeling,” Proteins: Struct. Funct. Bioinf. 68, 568-580; Masetti et al., 2007, “Modeling the hERG Potassium Channel in a Phospholipid Bilayer: Molecular Dynamics and Drug Docking Studies, J. Comp. Chem., 29(5), 795-808; Zachariae et al., 2009, “Side Chain Flexibilities in the Human Ether-a-go-go Related Gene Potassium Channel (hERG) Together with Matched-Pair Binding Studies Suggest a New Binding Mode for Channel Blockers,” J. Med. Chem., 52, 4266-4276; Boukharta et al., 2011, “Computer Simulations of Structure—Activity Relationships for hERG Channel Blockers,” Biochemistry, 50, 6146-6156; Durdagi et al., 2011, “Combined Receptor and Ligand-Based Approach to the Universal Pharmacophore Model Development for Studies of Drug Blockade to the hERG1 Pore Domain,” J. Chem. Inf. Model., 51, 463-474, Barakat et al., 2014, “A Human Ether-á-go-go-related (Herg) Ion Channel Atomistic Model Generated by Long Supercomputer Molecular Dynamics Simulations and its Use in Predicting Drug Cardiotoxicity,” Toxicol. Lett. 230(3), 382-392, Durdagi et al., 2012, “Modeling of open, closed, and open-inactivated states of the hERG1 channel: structural mechanisms of the state-dependent drug binding,” J. Chem. Inf. Model. 52(10), 2760-74). For example, the structure of the NaᵥAB bacterial sodium channel (2.7 Å resolution) has recently been determined (Payandeh et al., 2011, “The crystal structure of a voltage-gated sodium channel,” Nature 475, 353-359).

With respect to the hNav1.5 channel in particular, several receptor-based models of hNav1.5-drug interactions have been published (O'Reilly et al., 2012, “Bisphenol A binds to the local anesthetic receptor site to block the human cardiac sodium channel,” PLoS ONE, 7, e41667; Moreau et al., 2015, “Gating pore currents are defects in common with two Nav1.5 mutations in patients with mixed arrhythmias and dilated cardiomyopathy. J. Gen. Physiol., 145, 93-106). These models, however, did not include the extracellular loops, the intracellular domains as well as the voltage sensing domains (VSDs).

However, the lack of a complete 3D structure model of the hNaᵥ1.5 channel currently prevents screening drug candidates for activity against this cardiac ion channels. Although computational modeling methods may be employed to develop further insights into the structure-function relationships of this channel, the scarcity of X-ray crystal and/or NMR structures that can be used as templates employed in such modeling efforts has been a significant limiting factor in studying hNaᵥ structures.

In comparison, the recommended in vitro drug screening process of cardiac ion channels includes traditional patch clamp techniques, radiolabeled drug binding assays, 86RB-flux assays, and high-throughput cell-based fluorescent dyes and stably transfected hERG1 ion channels from Chinese hamster ovary (CHO) cells (Stork et al., 2007, “State Dependent Dissociation of HERG Channel Inhibitors,” Br. J. Pharmacol., 151, 1368-1376) and HEK 293 cells (also known as 293T cells) (Diaz et al., 2004, “The [³H]Dofetilide Binding Assay is a Predictive Screening Tool for hERG Blockade and Proarrhythmia: Comparison of Intact Cell and Membrane Preparations and Effects of Altering [K⁺]ₒ,” J. Pharmacol. Toxicol. Methods., 50(3), 187-199). Although elaborate nonclinical tests display a reasonable sensitivity and establish safety standards for novel therapeutics, the screening of all of potential candidates remains very time-consuming and thus increases the final cost of drug design.

What is therefore needed is an improved atomistic approach to the screening of drug candidates for their ability to block hNaᵥ1.5, including a complete structural model for the hNaᵥ1.5 channel, that addresses the state-dependent mechanism of action of blockers, without adding unnecessary complexity and/or costs.

### 3. SUMMARY

The benefits of simplified screening calculations centered around identified key residues in the permeation pore of hNaᵥ1.5 channel include, for example, faster and more accurate screening of potential drug candidates with respect to their ability to block hNaᵥ1.5.

Accordingly, provided herein are computational dynamic models of a membrane-bound ion channel, the human cardiac sodium ion channel (hNaᵥ1.5), that provide atomistic detailed sampling of the physiologically relevant conformational states of this channel. In certain embodiments, the model is combined with an atomistic detailed high throughput screening algorithm of test compounds in silico to predict cardiotoxicity or risk of cardiotoxicity and to select for compounds with reduced risk of cardiotoxicity. In certain preferred embodiments, the model of the hNaᵥ1.5 channel is the closed state of the channel.

In certain embodiments, the model and methods disclosed herein can be used to screen a standardized panel of drugs showing that cardiotoxic compounds are blockers of hNaᵥ1.5, whereas proven safe drugs do not block these channels. In certain embodiments, blockers are identified by comparing their binding energies to the binding energy of known blockers of the hNaᵥ1.5 channel. In certain embodiments, the model and methods disclosed herein can be used to screen thousands of new candidate drugs in silico, which greatly accelerates drug development and renders it safer and cheaper rather than having to test all compounds in biological assays.

In certain embodiments, the model and methods disclosed herein can be used to identify key residues of the hNaᵥ1.5 channel's ion permeation pathways. In certain embodiments, the model and methods includes steered molecular dynamics simulations of the hNaᵥ1.5 channel and an ion to identify these key residues. In certain embodiments of the steered molecular dynamics simulation, the ion is pulled through the permeation pathways by using an external force. In certain embodiments, dominant conformations of this channel around these key residues are identified which may be physiologically relevant in blocking the ion channel. In certain embodiments, the identified dominant conformations are selected for docking studies of compounds that cardiotoxic or are potentially cardiotoxic.

In certain embodiments, a key residue of the hNaᵥ1.5 channel's ion permeation pathways is selected from the group consisting of Phe892, Phe934, Phe1418, Phe1459, Phe1465, and Phe1760. In certain embodiments, the key residue is Phe1760. In certain embodiments, the key residue is a phenylalanine residue.

In certain embodiments, the model and methods disclosed herein can be used to predict compounds that are cardiotoxic or are potentially cardiotoxic, or to identify which chemical moieties of the compounds may be implicated in the toxicity, so that drug developers may avoid using the molecule, or may structurally modify the molecule to address the toxicity concerns. In certain embodiments, a plurality of preferred binding conformations is identified for each combination of the hNaᵥ1.5 channel and a compounds, the conformers of which are docked to the selected dominant conformations of the hNaᵥ1.5 channel.

In certain embodiments, the hNaᵥ1.5 channel used in the computational dynamic model is a transmembrane protein, surrounded by a membrane, ions, solvent or physiological fluid molecules, and optionally, other components of an in vivo system, to simulate the realistic environment of the channel. In certain embodiments, the duration of the computational dynamical model is of sufficient length (e.g., greater than 100 ns) to allow sampling of the dominant conformations of the hNaᵥ1.5 channel around the identified key residues. In certain embodiments, the coordinates of the model of the hNaᵥ1.5 channel are provided, including, for example, the coordinates listed in Table A.

In certain embodiments, the atomistic detail afforded by the computational dynamical model and high throughput screening algorithm allows a determination of whether a test compound has a lower or higher binding energy compared to the binding energy of a known blocker in the preferred binding conformations of the hNaᵥ1.5 channel. In certain embodiments, a test compound that has a lower, or equal, binding energy compared to the binding energy of a known blocker in the preferred conformations of the hNaᵥ1.5 channel is cardiotoxic. In certain embodiments, the calculation of binding energies uses MD simulations and molecular mechanics based on generalized Born and surface area solvation (MM-GBSA).

Certain embodiments, provided herein, include a system and methods for selecting a compound with reduced risk of cardiotoxicity. In certain embodiments, the system and method include a computational dynamic model combined with a high throughput screening in silico that mimics the closed conformational states of the hNaᵥ1.5 channel. Certain embodiments, also provided herein, include processor-implemented systems and methods for redesigning compounds that are predicted to be cardiotoxic based on the model and the high throughput screening.

Certain embodiments, provided herein, include providing an IC₅₀ values for each of the combination of the cardiac ion channel protein and the compound using the calculated binding energy of the compound and a function correlating IC₅₀ values and binding energies of compounds known to bind to the cardiac ion channel protein.

Certain embodiments, if the compound is predicted to be cardiotoxic, further include using a molecular modeling algorithm to chemically modify the compound such that the calculated binding energy of the modified compound in the preferred binding conformations exceeds the binding energy of the known blocker of the cardiac ion channel protein.

In certain embodiments, a processor-implemented system and methods include the steps of: a) building a plurality of homology models of a closed state of a cardiac ion channel protein from structural information of the cardiac ion channel protein; b) performing a molecular dynamics (MD) simulation of the plurality of homology models; c) selecting one or more structures from the MD simulation; d) performing a steered molecular dynamics (SMD) simulation of the one or more selected structures and an ion to identify key residues of the cardiac ion channel protein's ion permeation pathways; e) using a clustering algorithm to identify dominant conformations of the selected structure around the identified key residues of step d); f) selecting the dominant conformations identified from the clustering algorithm; g) providing structural information describing conformers of the compound; h) using a docking algorithm to dock the conformers of the compound of step f) to the dominant conformations of step e); i) identifying a plurality of preferred binding conformations for each of the combinations of the selected structure and the compound; and j) calculating a binding energy of the compound using constrained MD simulations of the preferred binding conformations of step i); wherein if the calculated binding energy of the compound in the preferred binding conformations is equal or less than the binding energy of a known blocker of the cardiac ion channel protein, the compound is predicted to be cardiotoxic; or wherein if the calculated binding energy of the compound in the preferred binding conformations exceeds the binding energy of the known blocker of the cardiac ion channel protein, the compound is predicted to have reduced risk of cardiotoxicity; and wherein based on the prediction that the compound has reduced risk of cardiotoxicity, the compound is selected.

In certain embodiments, steps a) through j) are executed on one or more processors. In certain embodiments, one or more of the steps a) through j) are not necessarily executed in the above recited order. In certain embodiments, one or more of the steps a) through j) of the method are performed in the above recited order. In certain embodiments, steps a) through j) are executed for a chemically modified compound.

In certain embodiments, the step a) includes performing Iterative Threading Assembly Refinement (I-Tasser) to comparative model individual subdomains of the homology models. In certain embodiments, step a) further includes assembling models of the individual subdomains using the Smith-Waterman local alignment algorithm. In certain embodiments, step a) includes refining the homology models using fragment-guided MD simulations.

In certain embodiments, the structural information of step a) is a three-dimensional (3D) structure. In certain embodiments, the structural information of step a) is an X-ray crystal structure, an NMR solution structure, or a model determined using a protein structure prediction algorithm. In certain embodiments, step a) includes providing a X-ray crystal structure, an NMR solution structure, or a model determined using a protein structure prediction algorithm. In certain embodiments, step a) includes providing a X-ray crystal structure or an NMR solution structure, wherein one or more of the homology models is entirely based on the X-ray crystal structure or the NMR solution structure. In certain embodiments, the protein structure prediction algorithm comprises application of I-Tasser.

In certain embodiments, the structural information of step a) is subjected to energy minimization (EM) prior to performing the MD simulation of step b) or the SMD simulation of step d). In certain embodiments, the EM includes restraining selected atoms of the homology models to positions prior to the EM. In certain embodiments, the structural information of step a) is subjected to one or more restrained MD simulations to equilibrate the homology models.

In certain embodiments, the cardiac ion channel protein is a membrane-bound protein or the cardiac ion channel protein is a voltage-gated or the cardiac ion channel protein is a sodium ion channel protein, and the sodium ion channel protein is hNaᵥ1.5.

In certain embodiments, the MD simulation of step b) or the SMD simulation of step d) incorporates implicit or explicit solvent molecules and ion molecules. In certain embodiments, the MD simulation of step b) or the SMD simulation of step d) incorporates a hydrated lipid bilayer with explicit phospholipid, solvent and ion molecules. In certain embodiments, the MD simulation of step b) or the SMD simulation of step d) uses an AMBER force field, a CHARMM force field, or a GROMACS force field.

In certain embodiments, the duration of the MD simulation of step b) or the SMD simulation of step d) is greater than 100 ns. In certain embodiments, the duration of the MD simulation is 100, 150, 200, 250, 300, 350, 400, 450 or 500 ns. In certain embodiments, the duration of the MD simulation of step b) is 580 ns and/or the duration of the SMD simulation of step d) is 200 ns. In certain embodiments, the MD simulation of step b) or SMD simulation of step d) uses NAMD software.

In certain embodiments, the one or more selected structures of step c) are selected from structures among a trajectory of the MD simulation.

In certain embodiments, the SMD simulation of step d) uses an external force to pull the ion trough the permeation pathways. In certain embodiments, the external force depends on a force constant and a pulling speed of the ion. In certain embodiments, the force constant is 1 kcal/mol, 2 kcal/mol, 3 kcal/mol, 4 kcal/mol, or 5 kcal/mol, and in certain embodiments, the pulling speed is constant and 0.25 Å/ps, 0.3 Å/ps, 0.35 Å/ps, 0.4 Å/ps, 0.45 Å/ps, or 0.5 Å/ps.

In certain embodiments, the docking algorithm of step h) is selected from DOCK, AutoDock, or Glide-SP.

In certain embodiments, based on the prediction that a compound has reduced risk of cardiotoxicity, the compound is selected for further development or possible use in humans, or to be used as a compound for further drug design. In certain embodiments of the methods disclosed herein, if the compound is predicted cardiotoxic, the method further comprises the step of using a molecular modeling algorithm to chemically modify or redesign the compound such that it has reduced risk of cardiotoxicity in any of the preferred binding conformations. In another aspect, provided herein is a method for chemically modifying a compound that is predicted to be cardiotoxic.

In another aspect, provided herein are biological methods for testing the cardiotoxicity of the compound or modified compound in an in vitro biological assay or in vivo in a wild type animal or a transgenic animal model.

In certain embodiments, the method further comprises testing the cardiotoxicity of the compound or modified compound in an in vitro biological assay. In certain embodiments, the in vitro biological assay comprises high throughput screening of ion channel and transporter activities. In certain embodiments, the in vitro biological assay comprises high throughput screening of sodium ion channel and transporter activities. In certain embodiments, the in vitro biological assay is a hNav1.5 channel inhibition assay. In certain embodiments, the in vitro biological assay comprises electrophysiology measurements in single cells. In certain embodiments, the electrophysiology measurements in single cells comprise patch clamp measurements.

In another aspect, provided herein is a processor-implemented system is provided for designing a compound in order to reduce risk of cardiotoxicity. The system includes one or more computer-readable mediums, a grid computing system, and a data structure. The one or more computer-readable mediums are for storing protein structural information representative of a cardiac ion channel protein and for storing compound structural information describing conformers of the compound. The grid computing system includes a plurality of processor-implemented compute nodes and a processor-implemented central coordinator, said grid computing system receiving the stored protein structural information and the stored compound structural information from the one or more computer-readable mediums. Said grid computing system uses the received protein structural information to perform molecular dynamics simulations for determining configurations of target protein flexibility over a simulation length of greater than 100 ns, and to perform steered molecular dynamics simulations for identifying key residues of the cardiac ion channel protein's ion permeation pathways. The molecular dynamics simulations and the steered molecular dynamics simulations involve each of the compute nodes determining forces acting on an atom based upon an empirical force field that approximates intramolecular forces, where numerical integration is performed to update positions and velocities of atoms. The central coordinator forms trajectories of molecular dynamics or steered molecular dynamics simulations based upon the updated positions and velocities of the atoms as determined by each of the compute nodes. Said grid computing system configured to: the molecular dynamic trajectories into one or more dominant conformations of the cardiac ion channel protein around the identified key residues, execute a docking algorithm that uses the compound's structural information in order to dock the compound's conformers to the one or more dominant conformations of the protein, and identify a plurality of preferred binding conformations for each of the combinations of protein and compound based on information related to the docked compound's conformers. The data structure is stored in memory which includes information about the one or more of the identified plurality of preferred binding conformations of the closed state of the cardiac ion channel protein. Based on a binding energy calculated from the information about the one or more of the identified plurality of preferred binding conformations, the compound is redesigned in order to reduce risk of cardiotoxicity.

In another aspect, provided herein, is a computer-implemented system for selecting a compound with reduced risk of cardiotoxicity which includes one or more data processors and a computer-readable storage medium encoded with instructions for commanding the one or more data processors to execute certain operations. The operations include: a) building a plurality of homology models of a closed state of a cardiac ion channel protein from structural information of the cardiac ion channel protein; b) performing a molecular dynamics (MD) simulation of the plurality of homology models; c) selecting one or more structures from the MD simulation; d) performing a steered molecular dynamics (SMD) simulation of the one or more selected structures and an ion to identify key residues of the cardiac ion channel protein's ion permeation pathways; e) using a clustering algorithm to identify dominant conformations of the selected structure around the identified key residues of step d); f) selecting the dominant conformations identified from the clustering algorithm; g) providing structural information describing conformers of one or more compounds; h) using a docking algorithm to dock the conformers of the one or more compounds of step f) to the dominant conformations of step e); i) identifying a plurality of preferred binding conformations for each of the combinations of the selected structure and compound; and j) calculating a binding energy of the compound using constrained MD simulations of the preferred binding conformations of step i). If the calculated binding energy of the compound in the preferred binding conformations is equal or less than the binding energy of a known blocker of the cardiac ion channel protein, the compound is predicted to be cardiotoxic. If the calculated binding energy of the compound in the preferred binding conformations exceeds the binding energy of the known blocker of the cardiac ion channel protein, the compound is predicted to have reduced risk of cardiotoxicity. Based on a prediction that the compound has reduced risk of cardiotoxicity, the compound is selected.

In another aspect, provided herein, is a non-transitory computer-readable storage medium is provided for storing a compound-selection program. The compound-selection program includes instructions, which when executed by a processor of a data processing system, cause the data processing system to: a) build a plurality of homology models of a closed state of a cardiac ion channel protein from structural information of the cardiac ion channel protein; b) perform a molecular dynamics (MD) simulation of the plurality of homology models; c) select one or more structures from the MD simulation; d) perform a steered molecular dynamics (SMD) simulation of the one or more selected structures and an ion to identify key residues of the cardiac ion channel protein's ion permeation pathways; e) use a clustering algorithm to identify dominant conformations of the selected structure around the identified key residues of step d); f) select the dominant conformations identified from the clustering algorithm; g) provide structural information describing conformers of one or more compounds; h) use a docking algorithm to dock the conformers of the one or more compounds of step f) to the dominant conformations of step e); i) identify a plurality of preferred binding conformations for each of the combinations of the selected structure and compound; and j) calculate a binding energy of the compound using constrained MD simulations of the preferred binding conformations of step i). If the calculated binding energy of the compound in the preferred binding conformations is equal or less than the binding energy of a known blocker of the cardiac ion channel protein, the compound is predicted to be cardiotoxic. If the calculated binding energy of the compound in the preferred binding conformations exceeds the binding energy of the known blocker of the cardiac ion channel protein, the compound is predicted to have reduced risk of cardiotoxicity. Based on a prediction that the compound has reduced risk of cardiotoxicity, the compound is selected.

In certain embodiments, the cardiac ion channel protein is a membrane-bound protein. In certain embodiments, the cardiac ion channel protein is voltage gated. In certain embodiments, the cardiac ion channel protein is a sodium ion channel protein. In certain embodiments, the sodium ion channel protein is hNav1.5.

In certain embodiments, the computer-implemented or processor-implemented systems and methods includes instead the step a) of using the coordinates of Table A describing a structure of a closed state of a cardiac ion channel protein.

### 4. BRIEF DESCRIPTION OF THE FIGURES

FIGS. 1A and 1B illustrate system block diagrams for selecting a compound that has reduced risk of cardiotoxicity, according to some embodiments of the present invention. Processes illustrated in the system block diagrams (1A) and (1B) are: Target Preparation (includes, e.g., combined de novo/homology protein modeling of hNaᵥ1.5), Ligand Collection Preparation (includes, e.g., translation of the 2D information of the ligand into a 3D representative structure), Ensemble Generation (includes, e.g., Molecular Dynamics simulations and clustering analysis), Identification of Key Permeation Pathway Residues (includes, e.g., Steered Molecular Dynamics simulations), Docking (includes, e.g., docking and scoring of docked ligand poses), MD Simulations on Top Scoring Poses (includes, e.g., Molecular Dynamics simulations), Rescoring using MM-PBSA (includes, e.g., binding free energy calculation and rescoring of top scoring ligand poses), and Experimental Testing (includes, e.g., a hNaᵥ1.5 channel inhibition assay). The top scoring ligand poses from the Rescoring step can act as positive controls for the next phase screening. The Ensemble Generation, Identification of Key Permeation Pathway Residues, Docking, MD Simulations on Top Scoring Poses, and Rescoring using MM-PBSA steps may be performed on a supercomputer, for example, the “IBM Blue Gene/Q” supercomputer system at the Health Sciences Center for Computational Innovation, University of Rochester (e.g., as shown in the block diagram (FIG. 1B)).

FIG. 2 illustrates a cartoon representation of the α and β-subunits of a complete VGSC, depicting all the four domains (Domain I-Domain IV), with each domain including a voltage-sensing domain (VSD) and a pore domain (PD), the positively charged S4 helical segments of the VSDs, which connect with the respective PD, and the location of inactivation gate, according to some embodiments of the present invention.

FIGS. 3A-3D illustrate the Ramachandran reports for the three models, according to some embodiments of the present invention.

FIGS. 4A and 4B illustrate a 3D structure for the complete hNaᵥ1.5 generated homology model, including the four domains, DI, DII, DIII and DIV, the location of the selectivity filter is shown as a wireframe surface, and the positioning of the domains/sub-domains relative to the cell membrane, in side view (FIG. 3A) and top view (FIG. 3B) views, according to some embodiments of the present invention.

FIG. 5 illustrates a top view of a 3D structure of a relaxed MD snapshot for the generated model of Naᵥ1.5, showing a sodium ion trapped within the inner selectivity filter in a region of negative potential, according to some embodiments of the present invention.

FIGS. 6A-6D illustrate 3D structures of the four domains (DI-DIV) forming the hNaᵥ1.5 ion channel, according to some embodiments of the present invention.

FIG. 7 illustrates a carton representation of the VSDI domain of the hNaᵥ1.5 model, including the S1-S4 helices, R222 forming a salt bridge with E161 and R225 pointing toward the central axis of the VSD (thereby likely effecting the gating current of hNaᵥ1.5), and the lower cluster of interacting residues, Y168, D179 and K225, which form a septum preventing the outward movement of waters and ions through the VSD pore, according to some embodiments of the present invention.

FIG. 8 illustrates the fluctuations of the atomic distances between sodium ion and the carboxylate carbon atoms of the negatively charged residues (Asp and Glu) in the outer selectivity filters during the 200-300 ns of classical MD simulations, according to some embodiments, of the present invention.

FIG. 9 illustrates a side view of a 3D structure of hNav1.5 channel model in unit cell of the MD simulation with phospholipid bilayer, waters of hydration, and ions, according to some embodiments of the present invention.

FIG. 10 illustrates an atomic RMSD plot for the Ca atoms of the trans-membrane region of hNav1.5 channel over the 580 ns MD simulation trajectory, according to some embodiments of the present invention.

FIGS. 11A-11C illustrate average electron density profiles of water molecules, lipid heads and lipid tails over the last 50 ns of the MD simulation (FIG. 11A), of water molecules at simulation times of 100 ns, 300 ns and 600 ns (FIG. 11B), and of sodium and chloride ions over the last 50 ns (FIG. 11C), across the membrane bilayer, according to some embodiments of the present invention.

FIGS. 12A and 12B illustrate top views of a 3D structure of the hNaᵥ1.5 model showing the arrangements of the selectivity filters (FIG. 8A) and the solvated sodium ion captured within the pore region (FIG. 8B), according to some embodiments of the invention.

FIG. 13 illustrates superimposed structures of the 20 dominant conformations for the hNaᵥ1.5 channel, selected through the RMSD clustering of binding site residues of the hNaᵥ1.5 MD trajectory, according to some embodiments of the present invention.

FIG. 14 illustrates pulling of Na+ ion out of the central cavity of the hNaᵥ1.5 model through a first permeation pathway, according to some embodiments of the invention.

FIGS. 15A and 15B illustrate the time-evolution of H-bond distances between the side chain oxygen atom of Asn1463 and the two hydrogen atoms of the side chain methyl groups in Leu938, according to some embodiment of the present invention.

FIG. 16 illustrates pulling of Na+ ion out of the central cavity of the Nav1.5 model through a first permeation pathway, according to some embodiment of the present invention.

FIG. 17 illustrates pulling of Na+ ion out of the central cavity of the Nav1.5 model through a first permeation pathway, according to some embodiment of the present invention.

FIG. 18 illustrates pulling of Na+ ion out of the central cavity of the hNaᵥ1.5 model through a second permeation pathway, according to some embodiments of the invention.

FIG. 19 illustrates pulling of Na+ ion out of the central cavity of the Nav1.5 model through a second permeation pathway, according to some embodiment of the present invention.

FIG. 20 illustrates pulling of Na+ ion out of the central cavity of the Nav1.5 model through a second permeation pathway, according to some embodiment of the present invention.

FIGS. 21A and 21B illustrate pulling of Na+ ion out of the central cavity of the hNaᵥ1.5 model through a first and second permeation pathway, according to some embodiments of the invention.

FIG. 22 illustrates RMSD clustering for binding site residues of the hNaᵥ1.5 model over the 580 MD production simulations, according to some embodiments of the present invention.

FIG. 23 illustrates a 2D scatter plot comparing the calculated AMBER-MMGBSA binding free energies against the in vitro measured pIC₅₀ data from CHEMBL, according to some embodiments of the present invention.

FIGS. 24A-24H illustrates 2D and 3D ligand interactions for four hNaᵥ1.5 blockers: (24A,24B) Ranolazine, (24C,24D) Lidocaine (24E,24F) Flecainide and (24G,24H) CHEMBL2012299, according to some embodiments of the present invention.

FIG. 25 illustrates a block diagram depicting an environment wherein users can interact with a grid computing environment, according to some embodiments of the present invention.

FIG. 26 illustrates a block diagram depicting hardware and software components for the grid computing environment, according to some embodiments of the present invention.

FIG. 27 illustrates a schematics of data structures utilized by a compound-selection system, according to some embodiments of the present invention.

FIG. 28 illustrates a block diagram depicting a compound-selection system provided on a stand-alone computer for access by a user, according to some embodiments of the present invention.

### 5. DETAILED DESCRIPTION

### 5.1 Definitions

As used herein, the term “cardiotoxic” or “cardiotoxicity” refers to having a toxic effect on the heart, for example, by a compound having a deleterious effect on the action of the heart, due to poisoning of the cardiac muscle or of its conducting system. In certain embodiments, long Q-T syndrome or “LQTS” is an aspect of cardiotoxicity.

As used herein, the term “reduced cardiotoxicity” refers to a favorable cardiotoxicity profile with reference to, for example, one or more ion channel proteins disclosed herein. In certain embodiments, a “ligand,” “compound” or “drug,” as defined herein, has reduced cardiotoxicity or regular physiological ion channel activity if it does not inhibit one or more ion channel proteins (e.g., sodium ion channel proteins, such as hNaᵥ1.5) disclosed herein. In certain embodiments, a ligand, compound or drug has reduced cardiotoxicity if it does not inhibit “hNaᵥ1.5.” In certain embodiments, a ligand, compound or drug has reduced cardiotoxicity if it does not inhibit the closed state of “hNaᵥ1.5.” In certain embodiments, a ligand, compound or drug has reduced cardiotoxicity if it is not a “blocker,” as defined herein. In certain embodiments, a ligand, compound or drug has reduced cardiotoxicity if it does not block, obstruct, or partially obstruct, the hNaᵥ1.5 channel, as defined herein. In certain embodiments, a ligand, compound or drug has reduced cardiotoxicity if it does not stabilize, block, obstruct, or partially obstruct, the closed state of hNaᵥ1.5 channel, as defined herein. In certain embodiments, a ligand, compound or drug has reduced cardiotoxicity if it is not a blocker of hNaᵥ1.5. In certain embodiments, a ligand, compound or drug has reduced cardiotoxicity if it is not a blocker of the closed state of hNaᵥ1.5.

As used herein, the terms “reducing risk” or “reduced risk” as it applies to cardiotoxicity (e.g., “reduced risk of cardiotoxicity”) refers to observable results which tend to demonstrate an improved cardiotoxicity profile with reference to, for example, the hNaᵥ1.5 channel. In certain embodiments, a ligand, compound or drug has a reduced risk of cardiotoxicity if it does not stabilize, block, obstruct, or partially obstruct, the hNaᵥ1.5 channel. In certain embodiments, a ligand, compound or drug has a reduced risk of cardiotoxicity if it does not stabilize, block, obstruct, or partially obstruct, the closed state of the hNaᵥ1.5 channel. In certain embodiments, a ligand, compound or drug has a reduced risk of cardiotoxicity if it does not stabilize, block, obstruct, or partially obstruct, the flow of Na⁺ ions through the hNaᵥ1.5 channel. In certain embodiments, a ligand, compound or drug has a reduced risk of cardiotoxicity if it is not a blocker of hNaᵥ1.5, particularly the closed state of the hNaᵥ1.5 channel. In certain embodiments, risk is reduced if there is at least about 10%, 20%, 30%, 40%, 50%, 60%, 70%, 80%, 90% or 100% decrease (as measured, e.g., by IC₅₀ data from in vitro biological assays) in the ability of the ligand, compound or drug to inhibit the hNaᵥ1.5 channel. In certain embodiments, a reduction in the risk of cardiotoxicity by at least about 90% indicates that cardiotoxicity has been eliminated with respect to the hNaᵥ1.5 channel. In certain embodiments, a ligand, compound or drug has a reduced risk of cardiotoxicity if its calculated binding energies, as defined herein, to the hNaᵥ1.5 channel, disclosed herein, compare to physiologically relevant concentrations of greater than or equal to 100 μM. In certain embodiments, a ligand, compound or drug has a reduced risk of cardiotoxicity if its “selectivity index (SI),” as defined herein, is greater than about 100, about 1000 or about 10,000.

As used herein, the term “LQTS” as used herein refers to long Q-T syndrome, a group of disorders that increase the risk for sudden death due to an abnormal heartbeat. The QT of LQTS refers to an interval between two points (Q and T) on the common electrocardiogram (ECG, EKG) used to record the electrical activity of the heart. This electrical activity, in turn, is the result of ions such as sodium and potassium passing through ion channels in the membranes surrounding heart cells. A prolonged QT interval indicates an abnormality in electrical activity that leads to irregularities in heart muscle contraction. One of these irregularities is a specific pattern of very rapid contractions (tachycardia) of the lower chambers of the heart called torsade de pointes, a type of ventricular tachycardia. The rapid contractions, which are not effective in pumping blood to the body, result in a decreased flow of oxygen-rich blood to the brain. This can result in a sudden loss of consciousness (syncope) and death.

As used herein, the term “lipid bilayer” refers to the basic structure of a cell membrane comprising a double layer of phospholipid molecules. Lipid bilayers are generally impermeable to ions (such as sodium ions).

As used herein, the term “hydrated lipid bilayer” refers to a lipid bilayer in the presence of water molecules.

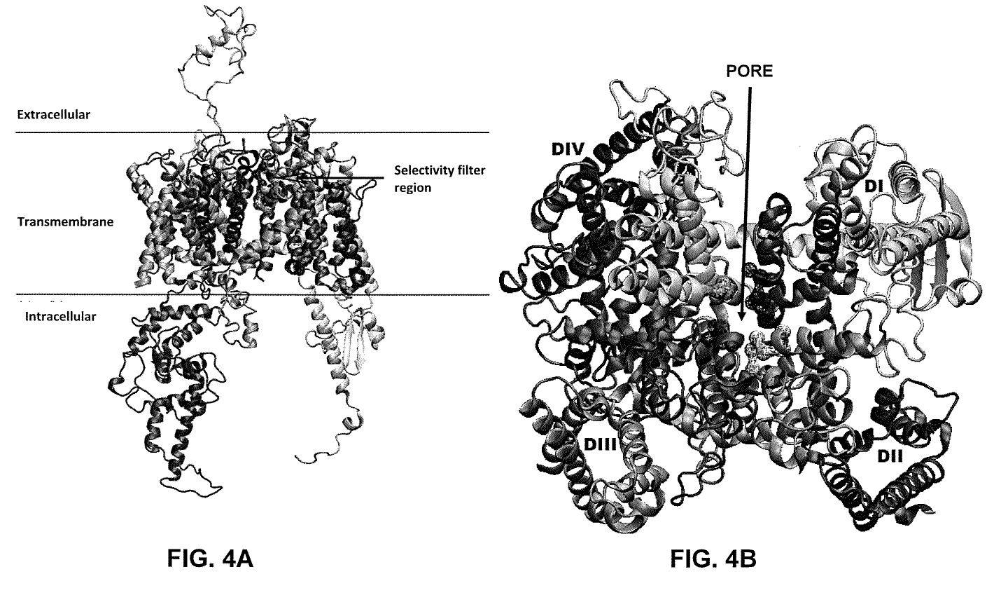

As used herein, the term “ion channel” or “ion channel protein,” refers to a membrane bound protein that acts as a pore (e.g., permeation pore) in a cell membrane and permits the selective passage of ions (such as sodium ions), by means of which electrical current passes in and out of the cell. Such ion channel proteins include, for example, sodium ion channel proteins, such as hNaᵥ1.5. In certain embodiments, an ion channel or ion channel protein comprises an inner cavity and a selectivity filter (see, e.g., FIGS. 4A and 4B) through which the ions pass. In certain embodiments, the terms “permeation pore,” “pore” and “channel” are used interchangeably.

As used herein, the term “gating,” when used in relation to an “ion channel” or “ion channel protein,” refers to the conformation change between closed, open and inactivated of the channel.

As used herein, the term “transporter activity,” when used in relation to an “ion channel” or “ion channel protein,” refers to the movement of an ion across a cell membrane.

As used herein, the term “sodium ion channel” or “sodium ion channel protein,” refers to an ion channel that permits the selective passage of sodium ions (Na⁺).

As used herein, the term “membrane bound protein” refers to any protein that is bound to a cell membrane under physiological pH and salt concentrations. In certain embodiments, binding of the membrane bound protein can be either by direct binding to the phospholipid bilayer or by binding to a protein, glycoprotein, or other intermediary that is bound to the membrane.

As used herein, the term “voltage-gated channel” or “voltage-gated ion channel” or “VGC” refers to a class of transmembrane ion channels that are activated by changes in electrical potential difference near the channel. In certain embodiments, the voltage-gated ion channel is a voltage-gated sodium channel.

As used herein, the term “voltage-gated sodium channel,” “voltage-gated sodium ion channel” or “voltage-gated sodium ion (Na⁺) channel” is a transmembrane channel specific for sodium and sensitive to voltage changes in the cell's membrane potential.

As used herein, the term “human Naᵥ1.5” or “hNaᵥ1.5” or refers to the sodium ion channel protein that in humans is encoded by the SCN5A gene. It will be known to those of ordinary skill in the art the functional hNaᵥ1.5 channel is comprised of single pore forming a subunit and ancillary β subunits, where the α subunit consists of four structurally homologous transmembrane domains designated DI-DIV, as disclosed herein.

As used herein, the term “protein structure” refers to the three-dimensional structure of a protein. The structure of a protein is characterized in four ways. The primary structure is the order of the different amino acids in a protein chain, whereas the secondary structure consists of the geometry of chain segments in forms such as helices or sheets. The tertiary structure describes how a protein folds in on itself; the quaternary structure of a protein describes how different protein monomers or monomer subunits fold in relation to each other.

As used herein, the term “monomer” or “monomer subunit” refers to one of the proteins making up the quaternary structure of a macromolecule.

As used herein, the term “hetero-tetramer” refers to a macromolecule, for example, a protein macromolecule, made up of four different monomer subunits or domains. An example of a hetero-tetramer is the hNaᵥ1.5 tetramer comprised of four domains (DI-IV). Tetrameric assembly into a quaternary structure is required for the formation of the functional hNaᵥ1.5 channel.

As used herein, the term “structural information” refers to the three dimensional structural coordinates of the atoms within a macromolecule, for example, a protein macromolecule such as hNaᵥ1.5.

As used herein, the term “three-dimensional (3D) structure” refers to the Cartesian coordinates corresponding to an atom's spatial relationship to other atoms in a macromolecule, for example, a protein macromolecule such as hNaᵥ1.5. Structural coordinates may be obtained using NMR techniques, as known in the art, or using x-ray crystallography as is known in the art. Alternatively, structural coordinates can be derived using molecular replacement analysis or homology modeling. Various software programs allow for the graphical representation of a set of structural coordinates to obtain a three dimensional representation of a molecule or molecular complex.

As used herein, the term “dynamics,” when applied to macromolecule and macromolecular structures, refers to the relative motion of one part of the molecular structure with respect to another. Examples include, but are not limited to: vibrations, rotations, stretches, domain motions, hinge motions, sheer motions, torsion, and the like. Dynamics may also include motions such as translations, rotations, collisions with other molecules, and the like.

As used herein, the term “flexible” or “flexibility,” when applied to macromolecule and macromolecular structures defined by structural coordinates, refers to a certain degree of internal motion about these coordinates, e.g., it may allows for bond stretching, rotation, etc.

As used herein, the term “molecular modeling algorithm” refers to computational approaches for structure prediction of macromolecule. For instance, these may comprise comparative protein modeling methods including homology modeling methods or protein threading modeling methods, and may further comprise ab initio or de novo protein modeling methods, or a combination of any such approaches.

As used herein, the term “computational dynamic model” refers to a computer-based model of a system that provides dynamics information of the system. In certain embodiments, when the system is a biological system, for example, a macromolecule or macromolecular structure, the computational dynamic model provides information of the vibrations, rotations, stretches, domain motions, hinge motions, sheer motions, torsion, translations, rotations, collisions with other molecules, and the like, exhibited by the system in the relevant time scale examined by the model.

As used herein, the term “molecular simulation” refers to a computer-based method to predict the functional properties of a system, including, for example, thermodynamic properties, thermochemical properties, spectroscopic properties, mechanical properties, transport properties, and morphological information. In certain embodiments, the molecular simulation is a molecular dynamics (MD) simulation.

As used herein, the term “molecular dynamics simulation” (MD or MD simulation) refers to computer-based molecular simulation methods in which the time evolution of a set of interacting atoms, groups of atoms or molecules, including macromolecules, is followed by integrating their equations of motion. The atoms or molecules are allowed to interact for a period of time, giving a view of the motion of the atoms or molecules. Thus, the MD simulations may be used to sample conformational space over time to predict the lowest energy, most populated, members of a conformational ensemble. Typically, the trajectories of atoms and molecules are determined by numerically solving the Newton's equations of motion for a system of interacting particles, where forces between the particles and potential energy are defined by molecular mechanics force fields. However, MD simulations incorporating principles of quantum mechanics and hybrid classical-quantum mechanics simulations are also available and may be contemplated herein.

As used herein, the term “scalable molecular dynamics” (scalable MD) refers to computational simulation methods which are suitably efficient and practical when applied to large situations (e.g., a large input data set, a large number of outputs or users, or a large number of participating nodes in the case of a distributed system). In certain embodiments, the methods disclosed herein use scalable MD for simulation of the large systems disclosed herein, for example, the hNaᵥ1.5 channel in a hydrated lipid bilayer with explicit phospholipid, solvent and ion molecules, free, or bound to ligand.

As used herein, the term “non-equilibrium steered molecular dynamics simulation” or “steered molecular dynamics simulation” (SMD or SMD simulation) refers to computer-based molecular simulation methods in which all the parameters and preparation for the SMD simulation are the same as those employed in a regular MD simulation, except for the introduction of an artificial external force in SMD, e.g., to pull an ion through the cavity of an ion channel. In the SMD simulation, a dummy atom is attached to the center of mass of a pulled atom included in the set of interacting atoms, groups of atoms or molecules in the SMD simulation using a virtual spring (with a spring constant of k). The dummy atom is subsequently pulled with a constant velocity v and in a selected direction \vec{n}. The numerical value of the pulling force at a given time F(t) can be estimated by the following equation:

F(t)=k[vt−(\vec{rₓ}−\vec{r₀})·\vec{n}] (1)

In equation (1), time t denotes the simulation time in pico seconds (ps), \vec{rₓ} is the current position of the pulled atom, e.g., the sodium ion within the central cavity of the hNaᵥ1.5 channel, and \vec{r₀} is the previous position of the pulled atom.

As used herein, the term “energy minimization” (EM) refers to computational methods for computing stable states of interacting atoms, groups of atoms or molecules, including macromolecules, corresponding to global and local minima on their potential energy surface. Starting from a non-equilibrium molecular geometry, EM employs the mathematical procedure of optimization to move atoms so as to reduce the net forces (the gradients of potential energy) on the atoms until they become negligible.

As used herein, the term “umbrella sampling” refers to computational methods used to improve the sampling of a system where an energy barrier separates two or more regions of conformational space. For example, umbrella sampling can be used to flatten the energy barrier between two regions of conformational space, allowing adequate sampling of the system.

As used herein, the term “potential of mean force” (PMF) is the potential that gives an average force over all the configurations of a given system.

As used herein, the term “potential of mean force simulation” (PMF or PFM simulation) is a type of simulation which is often used in Monte Carlo or MD simulations to examine how a system's energy changes as a function of some specific reaction coordinate parameter. For example, PMF simulations may be used to examine how the system's energy changes as a function of the distance between two residues, or as a protein is pulled through a lipid bilayer. One of ordinary skill in the art will understand that PMF simulations are used in conjunction with umbrella sampling because the PMF simulation will typically fail to adequately sample the system space as it proceeds.

As used herein, the term “ligand,” “compound” and “drug” are used interchangeably, and refer to any small molecule which is capable of binding to a target receptor, such as an ion channel protein, for example, hNaᵥ1.5. In certain embodiments, the ligand, compound or drug is a “blocker,” as defined herein. In some embodiments, a small molecule is a molecule having a molecular weight of less than 1,000 Da.

As used herein, the term “dock” or “docking” refers to using a model of a ligand and receptor to simulate association of the ligand-receptor at a proximity sufficient for at least one atom of the ligand to be within bonding distance of at least one atom of the receptor. The term is intended to be consistent with its use in the art pertaining to molecular modeling. A model included in the term can be any of a variety of known representations of a molecule including, for example, a graphical representation of its three-dimensional structure, a set of coordinates, set of distance constraints, set of bond angle constraints or set of other physical or chemical properties or combinations thereof. In certain embodiments, the ligand is a compound, for example a small molecule, and the receptor is a protein macromolecule, for example, hNaᵥ1.5.

As used herein, the term “docking algorithm” refers to computational approaches for predicting the energetically preferred orientation of a ligand to a receptor when bound or docked to each other to form a stable ligand-receptor complex. Knowledge of the preferred orientation in turn may be used to predict the strength of association or binding affinity between ligand and receptor using, for example, scoring functions. In certain embodiments, the ligand is a compound, for example a small molecule, and the receptor is a protein macromolecule, for example, hNaᵥ1.5.

As used herein, the term “drug design” or “rational drug design” refers to methods of processes of discovering new drugs based on the knowledge of a biological target. In certain embodiments of the methods disclosed herein, the biological target is a protein macromolecule, for example, hNaᵥ1.5. Those of ordinary skill in the art will appreciate that drug design that relies on the knowledge of the three-dimensional structure of the biomolecular target is also known as “structure-based drug design.” Those of ordinary skill in the art will also understand that drug design may rely on computer modeling techniques, which type of modeling is often referred to as “computer-aided drug design.” As used herein, the term “binding conformations” refers to the orientation of a ligand to a receptor when bound or docked to each other.

As used herein, the term “dominant conformation” or “dominant conformations” refers to most highly populated orientation(s) of a ligand to a receptor when bound or docked to each other. In certain embodiments, when applied to the trajectories of the MD simulations disclosed herein, a clustering algorithm is used to determine the “dominant conformation” or “dominant conformations.”

As used herein, the term “clustering algorithm,” when applied to a trajectory of the MD simulations disclosed herein, refers to computational approaches for grouping similar conformations in the trajectory into clusters.

As used herein, the term “preferred binding conformation” refers to the energetically preferred orientation of a ligand to a receptor when bound or docked to each other to form a stable ligand-receptor complex.

As used herein, the term “optimized preferred binding conformation” refers to the energetically preferred orientation of a ligand to a receptor when bound or docked to each other to form a stable ligand-receptor complex, following optimizing the preferred binding conformations using MD.

As used herein, the term “binding energies” is understood to mean the “free energy of binding” (ΔG^{o}) of a ligand to a receptor. Under equilibrium conditions, this binding energy is equal to ΔG^{o}=ΔH^{o}−T ΔS^{o}=−R T Log (K_{eq}), where the symbols have their customary meanings. In certain embodiments, the methods disclosed herein allow calculation of binding energies for various ligand-receptor complexes, for example, various compounds bound to hNaᵥ1.5.

As used herein, the terms “IC₅₀” refer to the concentration of a compound that reduces (e.g., inhibits) the enzyme activity of a target by 50%. The term “IC₅₀” generally describes the inhibitory concentration of the compound. Typically, measurements of IC₅₀ are made in vitro. In certain embodiments, where the target is a secondary biological target, for example, a membrane-bound ion channel implicated in cardiac cytotoxicity (e.g., hNaᵥ1.5), IC₅₀ is the concentration at which 50% inhibition is observed. IC₅₀'s can be measured according to any method known to one of ordinary skill in the art.

As used herein, the term “blocker” refers to a compound that blocks, obstructs, or partially obstructs, an ion channel, for example, the hNaᵥ1.5 ion channel. In certain embodiments, a blocker is a cardiotoxic compound.

As used herein, the term “non-blocker” refers to a compound that does not block, obstruct, or partially obstruct, an ion channel, for example, the hNaᵥ1.5 ion channel.

As used herein, “high throughput screening” refers to a method that allows a researcher to quickly conduct chemical, genetic or pharmacological tests, the results of which provide starting points for drug design and for understanding the interaction or role of a particular biochemical process in biology. In certain embodiments, the high throughput screening is through virtual in silico screening, for example, using computer-based methods or computer-based models.

As used herein, the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video discs (DVD), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

### 5.2 Embodiments

Provided herein is a computational dynamic model of the human cardiac sodium ion channel (hNaᵥ1.5) channel that provides an atomistic detailed sampling of the physiologically relevant conformational states of this channel, including the closed state. In certain embodiments, the model is combined with an atomistic detailed high throughput screening algorithm of test compounds in silico to predict cardiotoxicity and to select for compounds with reduced cardiotoxicity. In certain embodiments, key residues of the hNaᵥ1.5 channel's ion permeation pathways are identified and used to calculate in silico binding energies of the test compounds, which are compared to the binding energy of a known blocker of the hNaᵥ1.5 channel to predict their cardiotoxicity.

Human Nav1.5 (hNaᵥ1.5) is one of the most important cation channel that is responsible for initiating the myocardial action potential3. Diseases related mutations in Nav1.5 have been implicated in a wide range of cardiac diseases, such as long QT syndrome 3 (LQT3), Brugada syndrome 1 (BRGDA1) and sudden infant death syndrome (SIDS) (Campuzano et al., 2010; Remme, 2013). The α-subunit of hNav1.5 is a 220-kDa protein. Structurally, the hNaᵥ1.5 channel is arranged into four domains (DI-DIV) and is encoded by the SCN5A gene located on chromosome 3p21. Previously, no complete structural model for the human Nav1.5 channel existed due to the lack of experimental crystal and/or NMR structures that can be used as good templates.

In the computer-based molecular simulations and molecular models of hNaᵥ1.5 disclosed herein, the hNaᵥ1.5 structure is surrounded by a membrane, ions, and water molecules to simulate the realistic environment of the channel. Further, the computer-based molecular simulations disclosed herein are of sufficient length (e.g., greater than 100 ns) to allow sampling of physiologically relevant conformational states of the hNaᵥ1.5 channel, particularly the closed state. This robust molecular simulation of the hNaᵥ1.5 channel allows an atomistically detailed high throughput screening in silico to test compounds and determine if their calculated binding energies are equal or less to the one of a known blocker of the channel in any or all of the physiologically relevant conformational states, and therefore are likely to exhibit cardiotoxicity. The atomistic details of the molecular simulation also allow a chemical modification or redesign of those compounds found to be cardiotoxic. The redesigned compound may then be re-tested in an iterative fashion using the methods disclosed herein.

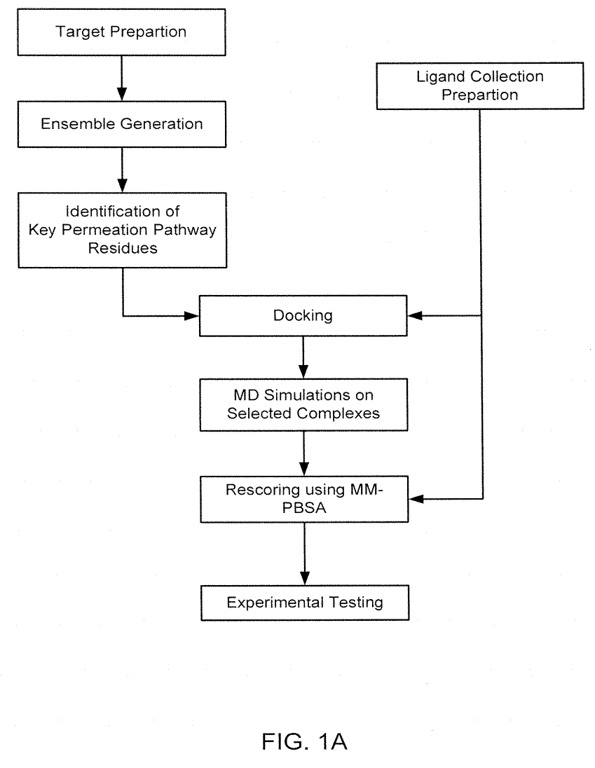

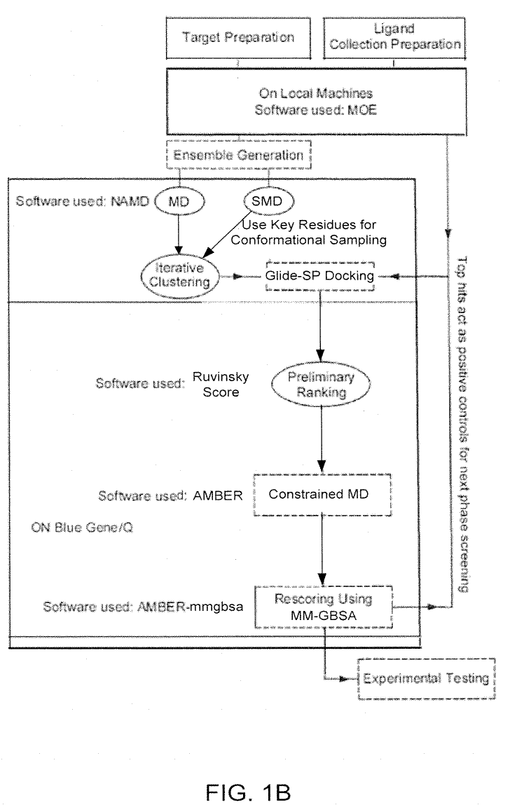

An overview of the methods disclosed herein, including computer-based molecular simulations and molecular models, is provided in FIGS. 1A and 1B. As an example, the methods can include: building a plurality of homology models of a closed state of a cardiac ion channel protein from structural information of the cardiac ion channel protein; performing a molecular dynamics (MD) simulation of the plurality of homology models; performing a molecular dynamics (MD) simulation of the plurality of homology models; selecting one or more structures from the MD simulation; performing a steered molecular dynamics (SMD) simulation of the one or more selected structures and an ion to identify key residues of the cardiac ion channel protein's ion permeation pathways; using a clustering algorithm to identify dominant conformations of the selected structure around the identified key residues; selecting the dominant conformations identified from the clustering algorithm; providing structural information describing conformers of the compound; using a docking algorithm to dock the compound conformers to the identified dominant conformations; identifying a plurality of preferred binding conformations for each of the combinations of the selected structure and the compound; and calculating a binding energy of the compound using constrained MD simulations of the preferred binding conformations of the previous step.

In certain embodiments, if the calculated binding energy of the compound in the preferred binding conformations is equal or less than the binding energy of a known blocker of the cardiac ion channel protein, the compound is predicted to be cardiotoxic. In certain embodiments, if the compound is predicted to be cardiotoxic, the compound is not selected, for example, for further clinical development or for use in humans. In certain embodiments, the compound may be structurally modified or redesigned to address cardiotoxicity.

In certain embodiments, if the calculated binding energy of the compound in the preferred binding conformations exceeds the binding energy of the known blocker of the cardiac ion channel protein, the compound is predicted to have reduced risk of cardiotoxicity. In certain embodiments, if the compound is predicted to have reduced risk of cardiotoxicity, the compound is selected, for example, for further development or possible use in humans, or to be used as a compound for further drug design.

Individual elements and steps of the methods disclosed herein are now described.

5.2.1 Ion Channels

In certain embodiments, the method comprises the step of using structural information describing the structure of a target receptor, for example, an ion channel protein. In certain embodiments, the target receptor is an ion channel that regulates cardiac function, for example, a cardiac ion channel disclosed herein. In certain embodiments, the cardiac ion channel is a membrane-bound protein. In certain embodiments, the cardiac ion channel is voltage-gated. In certain embodiments, the cardiac ion channel is a sodium ion channel.

Those of ordinary skill in the art will appreciate that ion channels, for example, a cardiac ion channel disclosed herein, may have two fundamental properties, ion permeation and gating. Ion permeation describes the movement through the open channel. The selective permeability of ion channels to specific ions is a basis of classification of ion channels (e.g., Na⁺ channels). Gating is the mechanism of opening and closing of ion channels. Voltage-dependent gating is the most common mechanism of gating observed in ion channels.

In certain embodiments, the ion channel is the hNaᵥ1.5 voltage gated sodium channel, as described below.

5.2.2 Human Naᵥ1.5 Voltage Gated Sodium Channel

The hNaᵥ1.5 channel as part of voltage gated sodium ion channels (VGSCs) is responsible for initiating the myocardial action potential and blocking Naᵥ1.5 through either mutations or its interactions with small molecule drugs or toxins have been associated with a wide range of cardiac diseases. These diseases include long QT syndrome 3 (LQT3), Brugada syndrome 1 (BRGDA1) and sudden infant death syndrome (SIDS). The DNA and amino acid sequences for hNaᵥ1.5 are provided as SEQ ID NO: 1 and SEQ ID NO: 2, respectively.

A detailed atomic structure of the hNaᵥ1.5 gene product based on X-ray cystallography or NMR spectroscopy is not yet available, so structural details for hNaᵥ1.5 are based on analogy with other ion channels, computer homology models, pharmacology, and mutagenesis studies. The structural information useful for the methods described herein is provided, for example, as a homology model, including wherein the homology model is represented by coordinates for a sodium ion channel protein (e.g., hNaᵥ1.5), as in Table A.

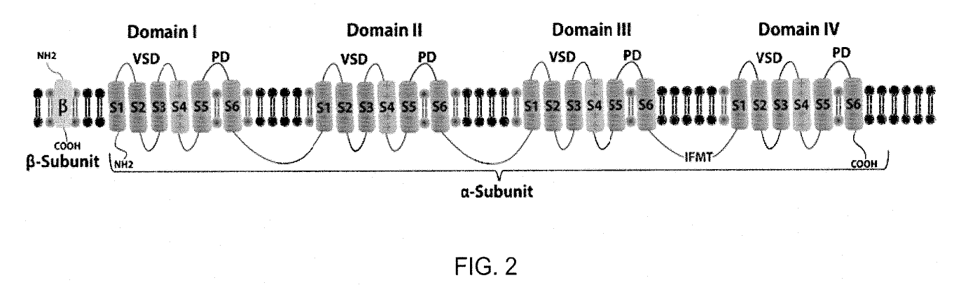

Eukaryotic VGSCs are hetero-tetramers in which the four domains (DI-IV; see FIG. 2) are different. DI comprises CYT1 (N-terminus) and TRM1, DII comprises TRM2, DIII comprises TRM3 and CYT4 (the inactivation gate), and DIV comprises TRM4 and CYT5 (C-terminus). The selectivity filter region as well as the selectivity specific residue in each TRM sub-domain are oriented inward toward the channel. Each TRM sub-domain is composed of six long helical segments (S1-S6). The first four segments (S1-S4) are grouped together in one side and are named as the voltage-sensing domain (VSD). The S4 segment is a 3₁₀ helix and is characterized by a highly conserved amino acid propensity of positively charged residues (Lys and Arg), usually called the “gating charges.” Some of these positively charged residues on S4 are held stabilized in the trans-membrane region through the formation of salt bridges with the negatively charged residues of S1-S3 (Asp and Glu) (Tiwari-Woodruff et al., 2000, “Voltage-Dependent Structural Interactions in the Shaker K(+) Channel,” J Gen Physiol 115: 123-138).

VGSCs generally share a common activation mechanism. A change in the membrane potential results in a conformational change and an outward movement of S4, allowing the activation of the channel and the passage of the captions through the channel's pore (Catterall, 2014, “Structure and Function of Voltage-Gated Sodium Channels at Atomic Resolution,” Exp Physiol 99: 35-51″). The last two helical segments from each domain (S5-S6) are usually referred to as the pore forming segments. The S5 helical segment is a long segment that extends horizontally from S4, through a linker, and then vertically through the trans-membrane region. A loop then connects S5 to two short helices named as the pore helices (P1 and P2). The S6 segment is connected to P2 through a short turn and extends vertically toward the intracellular part of the channel. A short turn connecting P1 and P2 contains the selectivity specific residues, which is uniquely conserved among VGSCs with the following arrangement (DEKA) splayed across the four domains and is known as the selectivity filter (D372, E898, K1419 and A1711). This DEKA selectivity filter is responsible for introducing the sodium selectivity over other mono/di-valent cations as has been shown previously by several experimental and computational mutational analyses (Lipkind et al., 2008, “Voltage-Gated Na Channel Selectivity: The Role of the Conserved Domain III Lysine Residue,” J Gen Physiol 131: 523-529). It has been shown that mutating the selectivity filter's residues not only affect the selectivity of the channel, but also the gating kinetics of the as well (Hilber, et al., 2005, “Selectivity Filter Residues Contribute Unequally to Pore Stabilization in Voltage-Gated Sodium Channels,” Biochemistry 44: 13874-13882).

Without being limited by any theory, in one aspect of the disclosure, the blocking of the central pore cavity or channel of hNaᵥ1.5 by a drug is a predictor of the cardiotoxicity of the drug. Undesired drug blockade of Na⁺ ion flux in hNaᵥ1.5 can lead to long QT syndrome, eventually inducing fibrillation and arrhythmia. Blockage of hNaᵥ1.5 is a significant problem experienced during the course of many drug discovery programs.

5.2.3 Computational Aspects

In certain aspects, provided herein are computational methods for selecting a compound that is not likely to be cardiotoxic.

In certain embodiments, the computational methods comprise a computational dynamic model. In certain embodiments, the computational dynamic model comprises a molecular simulation that samples conformational space over time. In certain embodiments, the molecular simulation is a molecular dynamics (MD) simulation.

In certain embodiments, the method comprising the steps of: a) building a plurality of homology models of a closed state of a cardiac ion channel protein from structural information of the cardiac ion channel protein; b) performing a molecular dynamics (MD) simulation of the plurality of homology models; c) selecting one or more structures from the MD simulation; d) performing a steered molecular dynamics (SMD) simulation of the one or more selected structures and an ion to identify key residues of the cardiac ion channel protein's ion permeation pathways; e) using a clustering algorithm to identify dominant conformations of the selected structure around the identified key residues of step d); f) selecting the dominant conformations identified from the clustering algorithm; g) providing structural information describing conformers of the compound; h) using a docking algorithm to dock the conformers of the compound of step f) to the dominant conformations of step e); i) identifying a plurality of preferred binding conformations for each of the combinations of the selected structure and the compound; and j) calculating a binding energy of the compound using constrained MD simulations of the preferred binding conformations of step i).

In certain embodiments, if the calculated binding energy of the compound in the preferred binding conformations is equal or less than the binding energy of a known blocker of the cardiac ion channel protein, the compound is predicted to be cardiotoxic.

In certain embodiments, if the calculated binding energy of the compound in the preferred binding conformations exceeds the binding energy of the known blocker of the cardiac ion channel protein, the compound is predicted to have reduced risk of cardiotoxicity

In certain embodiments, based on the prediction that the compound has reduced risk of cardiotoxicity, the compound is selected;

In certain embodiments, one or more of the steps a) through j) are not necessarily executed in the recited order. In certain embodiments, one or more of the steps a) through j) of the method are performed in the recited order.

In certain embodiments, step a) of the method instead of building a plurality of homology models of a closed state of a cardiac ion channel protein comprises using the coordinates of Table A describing a structure of a closed state of a cardiac ion channel protein.

In certain embodiments, steps b) through j) comprise a high-throughput screening of the compounds to determine if they are “cardiotoxic” or “have reduced risk of cardiotoxicity.”

In certain embodiments, steps a) through j) of the method are executed on one or more processors.

5.2.3.1 Structural Information of the hNaᵥ1.5 Channel

In certain embodiments, the method comprises the step of building a plurality of homology models of a closed state of a cardiac ion channel protein from structural information of the cardiac ion channel protein. In certain embodiments, the cardiac ion channel protein is the hNaᵥ1.5 channel. In certain embodiments, the cardiac ion channel protein is also referred to as a “receptor” or “target” and the terms “protein,” “receptor” and “target” are used interchangeably.

In certain embodiments, the method comprises the step of using the coordinates of Table A describing a structure of a closed state of a cardiac ion channel protein instead of building a plurality of homology models.

In certain embodiments, the structural information of the cardiac ion channel protein is from an NMR solution structure. Multidimensional heteronuclear NMR techniques for determination of the structure and dynamics of macromolecules are known to those of ordinary skill in the art (see, e.g., Rance et al., 2007, “Protein NMR Spectroscopy: Principles and Practice,” 2nd ed., Boston: Academic Press).

In certain embodiments, the structural information of the cardiac ion channel protein is from an X-ray crystal structure. X-ray crystallographic techniques for determination of the structure of macromolecules are also known to those of ordinary skill in the art (see, e.g., Drenth et al., 2007, “Principles of Protein X-Ray Crystallography,” 3rd ed., Springer Science).

In certain embodiments, a detailed atomic structure based on X-ray cystallography or NMR spectroscopy of the hNaᵥ1.5 channel is not yet available. Accordingly, structural details are based on analogy with other ion channels, computer homology models, pharmacology, and mutagenesis studies.

In certain embodiments, the step of building a plurality of homology models may comprise comparative protein modeling methods including homology modeling methods (see, e.g., Marti-Renom et al., 2000, Annu. Rev. Biophys. Biomol. Struct. 29, 291-325) performable without limitation using the “Modeller” computer program (Fiser and Sali, 2003, Methods Enzymol. 374, 461-91) or the “Swiss-Model” application (Arnold et al., 2006, Bioinformatics 22, 195-201); or protein threading modeling methods (see, e.g., Bowie et al., 1991, Science 253, 164-170; Jones et al., 1992, Nature 358, 86-89) performable without limitation using the “Hhsearch” program (Soding, 2005, Bioinformatics 21, 951-960), the “Phyre” application (Kelley and Sternberg, 2009, Nature Protocols 4, 363-371) or the “Raptor” program (Xu et al., 2003, J. Bioinform. Comput. Biol. 1, 95-117); may further comprise ab initio or de novo protein modeling methods using various algorithms, performable without limitation using the publically distributed “ROSETTA” platform (Simons et al., 1999, Genetics 37, 171-176; Baker 2000, Nature 405, 39-42; Bradley et al., 2003, Proteins 53, 457-468; Rohl 2004, Methods Enzymol. 383, 66-93), the “I-TASSER” application (Wu et al., 2007, BMC Biol. 5, 17), or using physics-based prediction (see, e.g., Duan and Kollman 1998, Science 282, 740-744; Oldziej et al., 2005, Proc. Natl. Acad. Sci. USA 102, 7547-7552); or a combination of any such approaches. Computational approaches applicable herein for structure prediction of biomolecules are evaluated annually within the Critical Assessment of Techniques for Protein Structure (CASP) experiment as published in the CASP Proceedings (http://predictioncenter.org/). Advantageously, data holding information about computationally predicted conformations and structures of many biomolecules such as peptides, polypeptides and proteins are available through respective publically available repositories (see, e.g., Kopp and Schwede, 2004, Nucleic Acids Research 32, D230-D234).

In certain embodiments, the methods disclosed herein work best with complex membrane-bound systems that are not susceptible to structure determination by X-ray crystallographic or NMR spectroscopic methods.

In certain embodiments, the structural information of the cardiac ion channel protein is selected from one or more of the closed, and/or open-inactivated structures of the bacterial NavAB bacterial sodium channel (Remme, 2013). In certain embodiments, existing X-ray crystal structures of the bacterial NavAB bacterial sodium channel were used as templates for the construction of TRM domains of the hNaᵥ1.5 channel, including, for example, crystal structures for the closed state (PDB IDs: 3RVY, 3RVZ and 3RWO) and inactivated state (PDB ID: 4EKW) of NavAB channel reported in the PDB database (Payandeh et al., 2011; Remme, 2013; McCusker et al., 2012, “Structure of a bacterial voltage-gated sodium channel pore reveals mechanisms of opening and closing,” Nat. Commun. 3, 1102; Bagneris et al., 2014, “Prokaryotic NavMs channel as a structural and functional model for eukaryotic sodium channel antagonism,” Proc. Natl. Acad. Sci. USA 111, 8428-8433).

5.2.3.2 Structural Information of the Compound (Ligand)

In certain embodiments, the method comprises providing structural information describing conformers of the compound. As used herein, the terms “compound” and “ligand” are interchangeable. In some embodiments, the structural information includes structural information generated from two-dimensional (2-D) description of the compound used to generate the structural information of the compound. An example of a 2-D description includes a SMILES string of the compound. In some embodiments, the structural information includes multiple conformations generated from the 2-D description of the compound.

One of ordinary skill in the art will understand that a chemical compound can adopt differing three-dimensional (3-D) shapes or “conformers” due to rotation of atoms about a bond. Conformers can thus interconvert by rotation around a single bond without breaking. A particular conformer of a ligand may provide a complimentary geometry to the pore (e.g., permeation pore) of an ion channel protein, and promote binding.

In certain embodiments, the structural information of describing conformers of one or more compounds or ligands is obtained from the chemical structure of a compound or ligand.

In certain embodiments, the structural information of the compound is based upon a viral compound being studied or developed by universities, pharmaceutical companies, or individual inventors. Typically, the compound will be a small organic molecule having a molecular weight under 900 atomic mass units. Structural information of the compound may be provided in 2D or 3D, using ChemDraw or simple structural depictions, or by entry of the compound's chemical name. Computer-based modeling of the compound may be used to translate the chemical name or 2D information of the compound into a 3D representative structure.

The software LigPrep from the Schrödinger package (Schrödinger Release 2013-2: LigPrep, version 2.7, Schrödinger, LLC, New York, N.Y., 2013) may be used to translate the 2D information of the compound (ligand) into a 3D representative structure which provides the structural information. LigPrep may also be used to generate variants of the same compound (ligand) with different tautomeric, stereochemical, and ionization properties. All generated structures may be conformationally relaxed using energy minimization protocols included in LigPrep.

In certain embodiments, the compound is selected from a list of compounds that have failed in clinical trials, or were halted in clinical trials due to cardiotoxicity.

In certain embodiments, the compound is selected from Table 1, below:

TABLE 1
Compounds Used For Cardiotoxicity Screening.

| Com- | | | |
|---|---|---|---|
| pound | | | CHEMBL |
| No. | Structure | Name | ID |
| 1 | 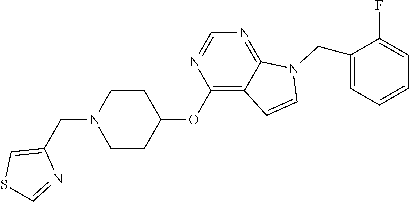 [SMILES: Fc1ccccc1Cn1ccc2c(OC3CCN(Cc4cscn4)CC3)ncnc21] | 4-((4-((7-(2- fluorobenzyl)-7H- pyrrolo[2,3- d]pyrimidin-4- yl)oxy)piperidin-1- yl)methyl)thiazole | CHEMBL 2012259 |
| 2 | 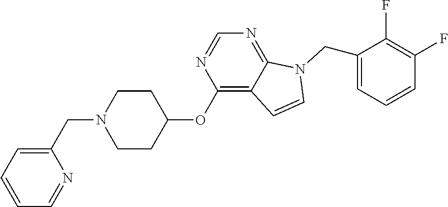 [SMILES: Fc1cccc(Cn2ccc3c(OC4CCN(Cc5ccccn5)CC4)ncnc32)c1F] | 7-(2,3- difluorobenzyl)-4- ((1-(pyridin-2- ylmethyl)piperidin- 4-yl)oxy)-7H- pyrrolo[2,3- d]pyrimidine | CHEMBL 2012299 |
| 3 | 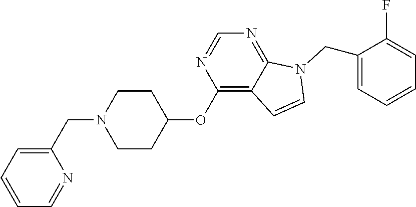 [SMILES: Fc1ccccc1Cn1ccc2c(OC3CCN(Cc4ccccn4)CC3)ncnc21] | 7-(2-fluorobenzyl)- 4-((1-(pyridin-2- ylmethyl)piperidin- 4-yl)oxy)-7H- pyrrolo[2,3- d]pyrimidine | CHEMBL 2012298 |
| 4 | 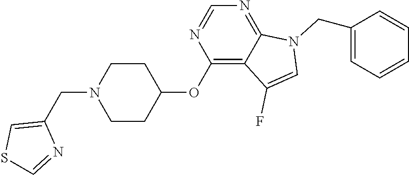 [SMILES: Fc1cn(Cc2ccccc2)c2ncnc(OC3CCN(Cc4cscn4)CC3)c12] | 4-((4-((7-benzyl-5- fluoro-7H- pyrrolo[2,3- d]pyrimidin-4- yl)oxy)piperidin-1- yl)methyl)thiazole | CHEMBL 2012180 |
| 5 | 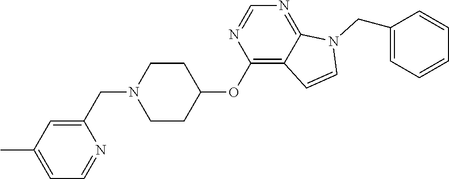 [SMILES: Cc1ccnc(CN2CCC(Oc3ncnc4c3ccn4Cc3ccccc3)CC2)c1] | 7-benzyl-4-((1-((4- methylpyridin-2- yl)methyl)piperidin- 4-yl)oxy)-7H- pyrrolo[2,3- d]pyrimidine | CHEMBL 2012292 |
| 6 | 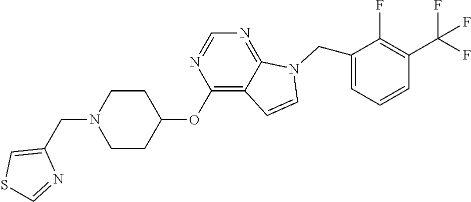 [SMILES: Fc1c(Cn2ccc3c(OC4CCN(Cc5cscn5)CC4)ncnc32)cccc1C(F)(F)F] | 4-((4-((7-(2-fluoro- 3- (trifluoromethyl) benzyl)-7H-pyrrolo [2,3-d]pyrimidin-4- yl)oxy)piperidin-1- yl)methyl)thiazole | CHEMBL 2012273 |
| 7 | 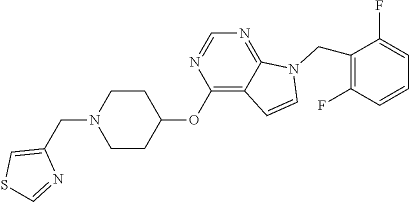 [SMILES: Fc1cccc(F)c1Cn1ccc2c(OC3CCN(Cc4cscn4)CC3)ncnc21] | 4-((4-((7-(2,6- difluorobenzyl)-7H- pyrrolo[2,3- d]pyrimidin-4- yl)oxy)piperidin-1- yl)methyl)thiazole | CHEMBL 2012274 |
| 8 | 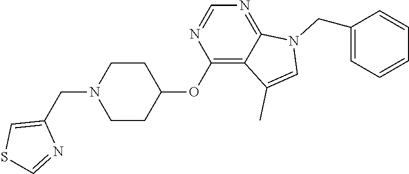 [SMILES: Cc1cn(Cc2ccccc2)c2ncnc(OC3CCN(Cc4cscn4)CC3)c12] | 4-((4-((7-benzyl-5- methyl-7H- pyrrolo[2,3- d]pyrimidin-4- yl)oxy)piperidin-1- yl)methyl)thiazole | CHEMBL 2012179 |
| 9 | 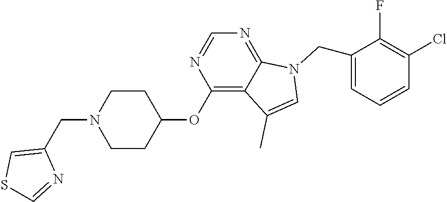 [SMILES: Cc1cn(Cc2cccc(Cl)c2F)c2ncnc(OC3CCN(Cc4cscn4)CC3)c12] | 4-((4-((7-(3-chloro- 2-fluorobenzyl)-5- methyl-7H- pyrrolo[2,3- d]pyrimidin-4- yl)oxy)piperidin-1- yl)methyl)thiazole | CHEMBL 2012272 |
| 10 | 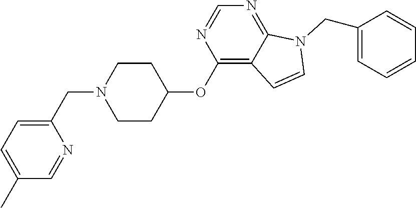 [SMILES: Cc1ccc(CN2CCC(Oc3ncnc4c3ccn4Cc3ccccc3)CC2)nc1] | 7-benzyl-4-((1-((5- methylpyridin-2- yl)methyl)piperidin- 4-yl)oxy)-7H- pyrrolo[2,3- d]pyrimidine | CHEMBL 2012291 |
| 11 | 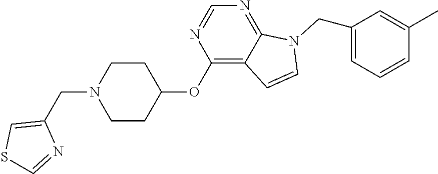 [SMILES: Cc1cccc(Cn2ccc3c(OC4CCN(Cc5cscn5)CC4)ncnc32)c1] | 4-((4-((7-(3- methylbenzyl)-7H- pyrrolo[2,3- d]pyrimidin-4- yl)oxy)piperidin-1- yl)methyl)thiazole | CHEMBL 2012266 |
| 12 | 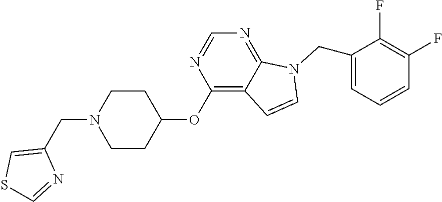 [SMILES: Fc1cccc(Cn2ccc3c(OC4CCN(Cc5cscn5)CC4)ncnc32)c1F] | 4-((4-((7-(2,3- difluorobenzyl)-7H- pyrrolo[2,3- d]pyrimidin-4- yl)oxy)piperidin-1- yl)methyl)thiazole | CHEMBL 2012271 |
| 13 | 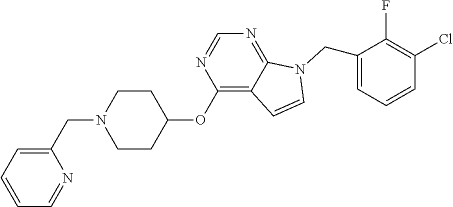 [SMILES: Fc1c(Cl)cccc1Cn1ccc2c(OC3CCN(Cc4ccccn4)CC3)ncnc21] | 7-(3-chloro-2- fluorobenzyl)-4-((1- (pyridin-2- ylmethyl)piperidin- 4-yl)oxy)-7H- pyrrolo[2,3- d]pyrimidine | CHEMBL 2012300 |
| 14 | 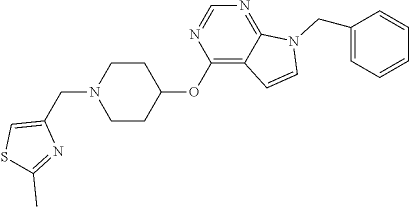 [SMILES: Cc1nc(CN2CCC(Oc3ncnc4c3ccn4Cc3ccccc3)CC2)cs1] | 4-((4-((7-benzyl- 7H-pyrrolo[2,3- d]pyrimidin-4- yl)oxy)piperidin-1- yl)methyl)-2- methylthiazole | CHEMBL 2012285 |
| 15 | 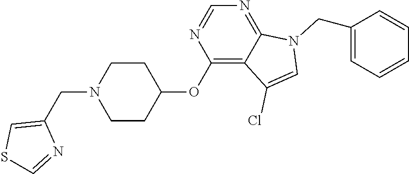 [SMILES: Clc1cn(Cc2ccccc2)c2ncnc(OC3CCN(Cc4cscn4)CC3)c12] | 4-((4-((7-benzyl-5- chloro-7H- pyrrolo[2,3- d]pyrimidin-4- yl)oxy)piperidin-1- yl)methyl)thiazole | CHEMBL 2012181 |
| 16 | 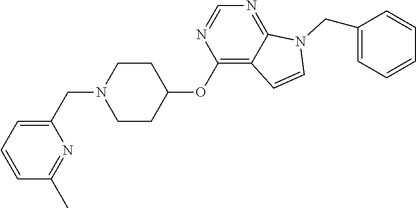 [SMILES: Cc1cccc(CN2CCC(Oc3ncnc4c3ccn4Cc3ccccc3)CC2)n1] | 7-benzyl-4-((1-((6- methylpyridin-2- yl)methyl)piperidin- 4-yl)oxy)-7H- pyrrolo[2,3- d]pyrimidine | CHEMBL 2012290 |
| 17 | 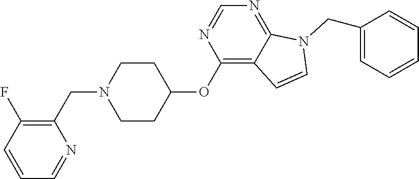 [SMILES: Fc1cccnc1CN1CCC(Oc2ncnc3c2ccn3Cc2ccccc2)CC1] | 7-benzyl-4-((1-((3- fluoropyridin-2- yl)methyl)piperidin- 4-yl)oxy)-7H- pyrrolo[2,3- d]pyrimidine | CHEMBL 2012296 |
| 18 | 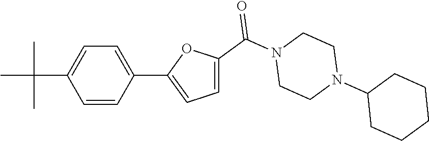 [SMILES: CC(C)(C)c1ccc(-c2ccc(C(=O)N3CCN(C4CCCCC4)CC3)o2)cc1] | (5-(4-(tert- butyl)phenyl)furan- 2-yl)(4- cyclohexyl- piperazin- 1-yl)methanone | CHEMBL 486495 |
| 19 | 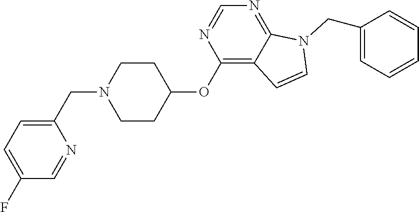 [SMILES: Fc1ccc(CN2CCC(Oc3ncnc4c3ccn4Cc3ccccc3)CC2)nc1] | 7-benzyl-4-((1-((5- fluoropyridin-2- yl)methyl)piperidin- 4-yl)oxy)-7H- pyrrolo[2,3- d]pyrimidine | CHEMBL 2012295 |
| 20 | 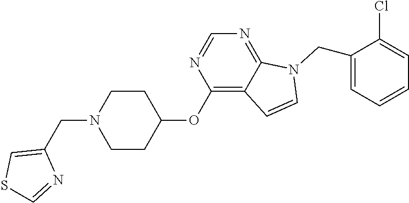 [SMILES: Clc1ccccc1Cn1ccc2c(OC3CCN(Cc4cscn4)CC3)ncnc21] | 4-((4-((7-(2- chlorobenzyl)-7H- pyrrolo[2,3- d]pyrimidin-4- yl)oxy)piperidin-1- yl)methyl)thiazole | CHEMBL 2012262 |
| 21 | 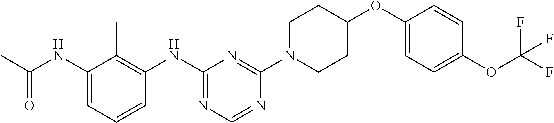 [SMILES: CC(=O)Nc1cccc(Nc2ncnc(N3CCC(Oc4ccc(OC(F)(F)F)cc4)CC3)n2)c1C] | N-(2-methyl-3-((4- (4-(4- (trifluoromethoxy) phenoxy)piperidin-1- yl)-1,3,5-triazin-2- yl)amino)phenyl) acetamide | CHEMBL 1813050 |
| 22 | 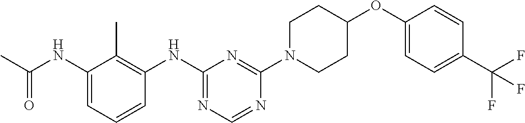 [SMILES: CC(=O)Nc1cccc(Nc2ncnc(N3CCC(Oc4ccc(C(F)(F)F)cc4)CC3)n2)c1C] | N-(2-methyl-3-((4- (4-(4- (trifluoromethyl) phenoxy)piperidin-1- yl)-1,3,5-triazin-2- yl)amino)phenyl) acetamide | CHEMBL 1813049 |
| 23 | 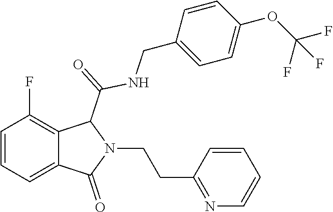 [SMILES: O=C(NCc1ccc(OC(F)(F)F)cc1)C1c2c(F)cccc2C(=O)N1CCc1ccccn1] | 7-fluoro-3-oxo-2-(2- (pyridin-2-yl)ethyl)- N-(4- (trifluoromethoxy) benzyl)isoindoline-1- carboxamide | CHEMBL 2164368 |
| 24 | 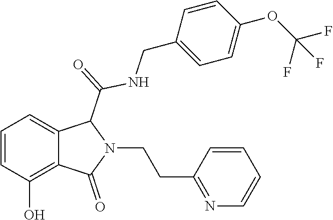 [SMILES: O=C(NCc1ccc(OC(F)(F)F)cc1)C1c2cccc(O)c2C(=O)N1CCc1ccccn1] | 4-hydroxy-3-oxo-2- (2-(pyridin-2- yl)ethyl)-N-(4- (trifluoromethoxy) benzyl)isoindoline-1- carboxamide | CHEMBL 2164041 |
| 25 | 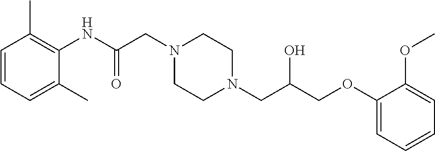 [SMILES: COc1ccccc1OCC(O)CN1CCN(CC(=O)Nc2c(C)cccc2C)CC1] | N-(2,6- dimethylphenyl)-2- (4-(2-hydroxy-3-(2- methoxyphenoxy) propyl)piperazin-1- yl)acetamide (Ranolazine) | |
| 26 | 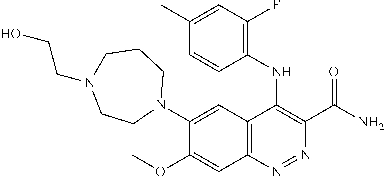 [SMILES: COc1cc2nnc(C(N)=O)c(Nc3ccc(C)cc3F)c2cc1N1CCCN(CCO)CC1] | 4-((2-fluoro-4- methylphenyl)amino)- 6-(4-(2- hydroxyethyl)-1,4- diazepan-1-yl)-7- methoxycinnoline- 3-carboxamide | CHEMBL 2409402 |
| 27 | 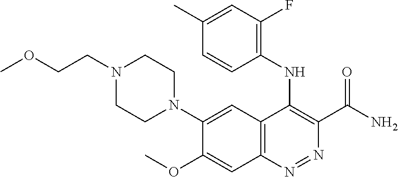 [SMILES: COCCN1CCN(c2cc3c(Nc4ccc(C)cc4F)c(C(N)=O)nnc3cc2OC)CC1] | 4-((2-fluoro-4- methylphenyl)amino)- 7-methoxy-6-(4- (2- methoxyethyl)piper- azin-1-yl)cinnoline- 3-carboxamide | CHEMBL 2409401 |
| 28 | 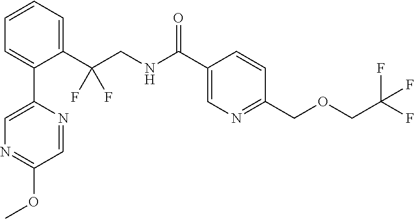 [SMILES: COc1cnc(-c2ccccc2C(F)(F)CNC(=O)c2ccc(COCC(F)(F)F)nc2)cn1] | N-(2,2-difluoro-2- (2-(5- methoxypyrazin-2- yl)phenyl)ethyl)-6- ((2,2,2- trifluoroethoxy) methyl)nicotinamide | CHEMBL 2147316 |
| 29 | 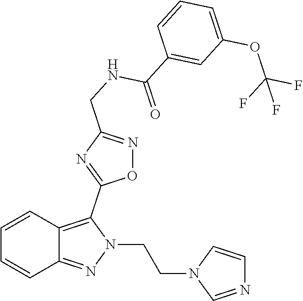 [SMILES: O=C(NCc1noc(-c2c3ccccc3nn2CCn2ccnc2)n1)c1cccc(OC(F)(F)F)c1] | N-((5-(2-(2-(1H- imidazol-1- yl)ethyl)-2H- indazol-3-yl)-1,2,4- oxadiazol-3- yl)methyl)-3- (trifluoromethoxy) benzamide | CHEMBL 3236329 |
| 30 | 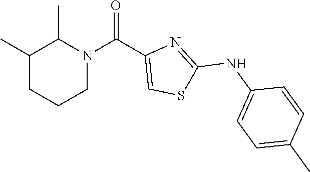 | (2,3- dimethylpiperidin-1- yl)(2-(p- tolylamino)thiazol- 4-yl)methanone | CHEMBL 2418806 |
| 31 | 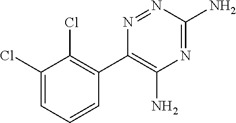 [SMILES: Nc1nnc(-c2cccc(Cl)c2Cl)c(N)n1] | 6-(2,3- dichlorophenyl)- 1,2,4-triazine-3,5- diamine | CHEMBL 741 |
| 32 | 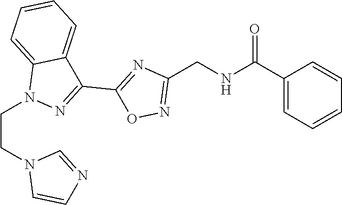 [SMILES: O=C(NCc1noc(-c2nn(CCn3ccnc3)c3ccccc23)n1)c1ccccc1] | N-((5-(1-(2-(1H- imidazol-1- yl)ethyl)-1H- indazol-3-yl)-1,2,4- oxadiazol-3- yl)methyl) benzamide | CHEMBL 3236343 |
| 33 | 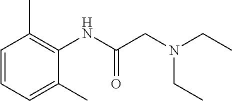 [SMILES: CCN(CC)CC(=O)Nc1c(C)cccc1C] | 2-(diethylamino)-N- (2,6- dimethylphenyl) acetamide (Lidocaine) | CHEMBL 79 |
| 34 | 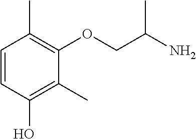 | 3-(2- aminopropoxy)-2,4- dimethylphenol | CHEMBL 2018453 |
| 35 | 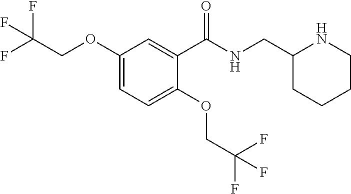 [SMILES: O=C(NCC1CCCCN1)c1cc(OCC(F)(F)F)ccc1OCC(F)(F)F] | N-(piperidin-2- ylmethyl)-2,5- bis(2,2,2- trifluoroethoxy) benzamide (Flecainide) | |

In certain embodiments, the compound is a known blocker of cardiac ion channel protein. In certain embodiments, the compound is a known blocker of the hNaᵥ1.5 channel. In certain embodiments, the compound is selected from the group consisting of: Ranolazine, Lidocaine, Flecainide and CHEMBL2012299 (Compound No. 2).

In certain embodiments, the compound is an anticancer agent, such as anthracyclines, mitoxantrone, cyclophosphamide, fluorouracil, capecitabine and trastuzumab. In certain embodiments, the compound is an immunomodulating drug, such as interferon-alpha-2, interleukin-2, infliximab and etanercept. In certain embodiments, the compound is an antidiabetic drug, such as rosiglitazone, pioglitazone and troglitazone. In certain embodiments, the compound is an antimigraine drug, such as ergotamine and methysergide. In certain embodiments, the compound is an appetite suppressant, such as fenfulramine, dexfenfluramine and phentermine. In certain embodiments, the compound is a tricyclic antidepressant. In certain embodiments, the compound is an antipsychotic drug, such as clozapine. In certain embodiments, the compound is an antiparkinsonian drug, such as pergolide and cabergoline. In certain embodiments, the compound is a glucocorticoid. In certain embodiments, the compound is an antifungal drugs such as itraconazole and amphotericin B. In certain embodiments, the compound is an NSAID, including selective cyclo-oxygenase (COX)-2 inhibitors.

In certain embodiments, the compound is selected from the group consisting of an antihistamine, an antiarrhythmic, an antianginal, an antipsychotic, an anticholinergic, an antitussive, an antibiotic, an antispasmodic, a calcium antagonist, an inotrope, an ACE inhibitor, an antihypertensive, a beta-blocker, an antiepileptic, a gastroprokinetic agent, an alphal-blocker, an antidepressant, an aldosterone antagonist, an opiate, an anesthetic, an antiviral, a PDE inhibitor, an antifungal, a serotonin antagonist, an antiestrogen, and a diuretic.

In certain embodiments, the compound is an active ingredient in a natural product. In certain embodiments, the compound is a toxin or environmental pollutant.

In certain embodiments, the compound is an antiviral agent.

In certain embodiments, the compound is selected from the group consisting of a protease inhibitor, an integrase inhibitor, a chemokine inhibitor, a nucleoside or nucleotide reverse transcriptase inhibitor, a non-nucleoside reverse transcriptase inhibitor, and an entry inhibitor.

In certain embodiments, the compound is selected from the group consisting of Abacavir, Aciclovir, Acyclovir, Adefovir, Amantadine, Amprenavir, Ampligen, Arbidol, Atazanavir, Balavir, Boceprevirertet, Cidofovir, Darunavir, Delavirdine, Didanosine. Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Imiquimod, Indinavir, Inosine, Interferon type III, Interferon type II, Interferon type I, Interferon, Lamivudine, Lopinavir, Loviride, Maraviroc, Moroxydine, Methisazone, Nelfinavir, Nevirapine, Nexavir, Oseltamivir (Tamiflu), Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

5.2.3.3 Energy Minimization

In certain embodiments, the X-ray crystal structure, NMR solution structures, homology models, molecular models, or generated structures disclosed herein are subjected to energy minimization (EM) prior to performing an MD simulation.

The goal of EM is to find a local energy minimum for a potential energy function. A potential energy function is a mathematical equation that allows the potential energy of a molecular system to be calculated from its three-dimensional structure. Examples of energy minimization algorithms include, but are not limited to, steepest descent, conjugated gradients, Newton-Raphson, and Adopted Basis Newton-Raphson (Molecular Modeling: Principles and Applications, Author A. R. Leach, Pearson Education Limited/Prentice Hall (Essex, England), 2^{nd} Edition (2001) pages: 253-302). It is possible to use several methods interchangeably.

5.2.3.4 Molecular Simulations

In certain embodiments, the method comprises the step of performing a molecular dynamics (MD) simulation of the plurality of homology models, and selecting one or more structures from the MD simulation. Accordingly, provided herein are molecular simulations that sample conformational space of the cardiac ion channel protein according to the methods described herein. In certain embodiments, the molecular simulation is a MD simulation.

Molecular simulations can be used to monitor time-dependent processes of the macromolecules and macromolecular complexes disclosed herein, in order to study their structural, dynamic, and thermodynamic properties by solving the equation of motion according to the laws of physics, e.g., the chemical bonds within proteins may be allowed to flex, rotate, bend, or vibrate as allowed by the laws of chemistry and physics. This equation of motion provides information about the time dependence and magnitude of fluctuations in both positions and velocities of the given molecule. Interactions such as electrostatic forces, hydrophobic forces, van der Waals interactions, interactions with solvent and others may also be modeled in MD simulations. The direct output of a MD simulation is a set of “snapshots” (coordinates and velocities) taken at equal time intervals, or sampling intervals. Depending on the desired level of accuracy, the equation of motion to be solved may be the classical (Newtonian) equation of motion, a stochastic equation of motion, a Brownian equation of motion, or even a combination (Becker et al., eds. Computational Biochemistry and Biophysics. New York 2001).

One of ordinary skill in the art will understand that direct output of a MD simulation, that is, the “snapshots” taken at sampling intervals of the MD simulation, will incorporate thermal fluctuations, for example, random deviations of a system from its average state, that occur in a system at equilibrium.

In certain embodiments, the molecular simulation is conducted using the CHARMM (Chemistry at Harvard for Macromolecular Modeling) simulation package (Brooks et al., 2009, “CHARMM: The Biomolecular Simulation Program,” J. Comput. Chem., 30(10):1545-614). In certain embodiments, the molecular simulation is conducted using the NAMD (Not (just) Another Molecular Dynamics program) simulation package (Phillips et al., 2005, “Scalable Molecular Dynamics with NAMD,” J. Comput. Chem., 26, 1781-1802; Kalé et al., 1999, “NAMD2: Greater Scalability for Parallel Molecular Dynamics,” J. Comp. Phys. 151, 283-312). One of skill in the art will understand that multiple packages may be used in combination. Any of the numerous methodologies known in the art to conduct MD simulations may be used in accordance with the methods disclosed herein. The following publications, which are incorporated herein by reference, describe multiple methodologies which may be employed: AMBER (Assisted Model Building with Energy Refinement) (Case et al., 2005, “The Amber Biomolecular Simulation Programs,” J. Comput. Chem. 26, 1668-1688; amber.scripps.edu); CHARMM (Brooks et al., 2009, J. Comput. Chem., 30(10):1545-614; charmm.org); GROMACS (GROningen MAchine for Chemical Simulations) (Van Der Spoel et al., 2005, “GROMACS: Fast, Flexible, and Free,” J. Comput. Chem., 26(16), 1701-18; gromacs.org); GROMOS (GROningen MOlecular Simulation) (Schuler et al., 2001, J. Comput. Chem., 22(11), 1205-1218; igc.ethz.ch/GROMOS/index); LAMMPS (Large-scale Atomic/Molecular Massively Parallel Simulator) (Plimpton et al., 1995, “Fast Parallel Algorithms for Short-Range Molecular Dynamics,” J. Comput. Chem., 117, 1-19; lammps.sandia.gov); and NAMD (Phillips et al., 2005, J. Comput. Chem., 26, 1781-1802; Kalé et al., 1999, J. Comp. Phys. 151, 283-312).

Wherein the methods call for a MD simulation, the simulation may be carried out using a simulation package chosen from the group comprising or consisting of AMBER, CHARMM, GROMACS, GROMOS, LAMMPS, and NAMD. In certain embodiments, the simulation package is the CHARMM simulation package. In certain embodiments, the simulation package is the NAMD simulation package.

Wherein the methods call for a MD simulation, the simulation may be of any duration. In certain embodiments, the duration of the MD simulation is greater than 100 ns. In certain embodiments, the duration of the MD simulation is greater than 200 ns. In certain embodiments, the duration of the MD simulation is greater than 300 ns. In certain embodiments, the duration of the MD simulation is greater than 150 ns. In certain embodiments, the duration of the MD simulation is greater than 100 ns. In certain embodiments, the duration of the MD simulation is greater than 50 ns. In certain embodiments, the duration of the MD simulation of step is about 50, 60, 70, 80, 90, 100, 110, 120, 130, 140, 150, 160, 170, 180, 190, 200, 210, 220, 230, 240, 250, 300, 350, 400, 450 or 500 ns.

In certain embodiments, the molecular simulation incorporates solvent molecules. In certain embodiments, the molecular simulation incorporates implicit or explicit solvent molecules. One of ordinary skill in the art will understand that implicit solvation (also known as continuum solvation) is a method of representing solvent as a continuous medium instead of individual “explicit” solvent molecules most often used in MD simulations and in other applications of molecular mechanics. In certain embodiments, the molecular simulation incorporates water molecules. In certain embodiments, the molecular simulation incorporates implicit or explicit water molecules. In certain embodiments, the molecular simulation incorporates explicit ion molecules. In certain embodiments, the molecular simulation incorporates a lipid bilayer. In certain embodiments, the lipid bilayer incorporates explicit lipid molecules. In certain embodiments, the lipid bilayer incorporates explicit phopholipid molecules. In certain embodiments, the lipid bilayer incorporates a solvated lipid bilayer. In certain embodiments, the lipid bilayer incorporates a hydrated lipid bilayer. In certain embodiments, the hydrated lipid bilayer incorporates explicit phospholipid molecules and explicit water molecules.

5.2.3.5 Steered Molecular Dynamics Simulation

In certain embodiments, the method comprises the step of performing a steered molecular dynamics (SMD) simulation of the structure of the cardiac ion channel protein. In certain embodiments, the method comprises the step of performing a SMD simulation of the one or more selected structures and an ion to identify key residues of the cardiac ion channel protein's ion permeation pathways. Accordingly, provided herein are steered molecular simulations that sample conformational space of the cardiac ion channel protein according to the methods described herein. In certain embodiments, the steered molecular simulation is a SMD simulation.

SMD simulations can be used to monitor time-dependent processes of the macromolecules and macromolecular complexes disclosed upon applying an external force to one of the macromolecule and/or macromolecular complex herein, in order to study their structural, dynamic, and thermodynamic properties by solving the equation of motion according to the laws of physics, e.g., the chemical bonds within proteins may be allowed to flex, rotate, bend, or vibrate as allowed by the laws of chemistry and physics. This equation of motion provides information about the time dependence and magnitude of fluctuations in both positions and velocities of the given molecule. Interactions such as electrostatic forces, hydrophobic forces, van der Waals interactions, interactions with solvent and others may also be modeled in SMD simulations. Accordingly, the parameters and preparation for the SMD simulation are the same as those employed in a regular MD simulation, except for the introduction of an artificial external force in SMD, e.g., to pull an ion through the cavity of an ion channel. In the SMD simulation, a dummy atom is attached to the center of mass of a pulled atom included in the set of interacting atoms, groups of atoms or molecules in the SMD simulation using a virtual spring (with a spring constant of k). The dummy atom is subsequently pulled with a constant velocity v and in a selected direction n. The numerical value of the pulling force at a given time F(t) can be estimated by the above equation (1).

In certain embodiments, t denotes the simulation time in pico seconds (ps), \vec{rₓ} is the current position of the pulled atom, e.g., the sodium ion within the central cavity of the hNaᵥ1.5 channel, and \vec{r₀} is the previous position of the pulled atom.

In certain embodiments, the constant velocity v and the spring constant k is v=0.25 Å/ps, 0.3 Å/ps, 0.35 Å/ps, 0.4 Å/ps, 0.45 Å/ps, or 0.5 Å/ps and k=1 kcal/mol, 2 kcal/mol, 3 kcal/mol, 4 kcal/mol, or 5 kcal/mol, respectively. In certain embodiments, values of constant velocity and spring constant are employed in the steered molecular simulation that do not induce any significant structural changes over shorter time intervals, e.g., 1-10 ps, in the system due to the external force.

The direct output of an SMD simulation is a set of “snapshots” (coordinates and velocities) taken at equal time intervals, or sampling intervals. Depending on the desired level of accuracy, the equation of motion to be solved may be the classical (Newtonian) equation of motion, a stochastic equation of motion, a Brownian equation of motion, or even a combination (Becker et al., eds. Computational Biochemistry and Biophysics. New York 2001). One of ordinary skill in the art will understand that direct output of an SMD simulation, that is, the “snapshots” taken at sampling intervals of the SMD simulation, will incorporate thermal fluctuations around a non-equilibrium state of the system.

In certain embodiments, the molecular simulation is conducted using the NAMD (Not (just) Another Molecular Dynamics program) simulation package (Phillips et al., 2005, “Scalable Molecular Dynamics with NAMD,” J. Comput. Chem., 26, 1781-1802; Kalé et al., 1999, “NAMD2: Greater Scalability for Parallel Molecular Dynamics,” J. Comp. Phys. 151, 283-312). One of a skill in the art will understand that multiple packages may be used in combination. Any of the numerous methodologies known in the art to conduct SMD simulations may be used in accordance with the methods disclosed herein. Wherein the methods call for an SMD simulation, the simulation may be carried out using a modified simulation package chosen from the group comprising or consisting of AMBER, CHARMM, GROMACS, GROMOS, LAMMPS, and NAMD, wherein the modification introduces an external force to the equations of motions. In certain embodiments, the simulation package is the NAMD simulation package.

Wherein the methods call for an SMD simulation, the simulation may be of any duration. In certain embodiments, the duration of the SMD simulation is greater than 100 ns. In certain embodiments, the duration of the SMD simulation is greater than 200 ns. In certain embodiments, the duration of the SMD simulation is greater than 300 ns. In certain embodiments, the duration of the SMD simulation is greater than 150 ns. In certain embodiments, the duration of the SMD simulation is greater than 100 ns. In certain embodiments, the duration of the SMD simulation is greater than 50 ns. In certain embodiments, the duration of the SMD simulation of step is about 50, 60, 70, 80, 90, 100, 110, 120, 130, 140, 150, 160, 170, 180, 190, 200, 210, 220, 230, 240, 250, 300, 350, 400, 450 or 500 ns.

In certain embodiments, the steered molecular simulation incorporates solvent molecules. In certain embodiments, the steered molecular simulation incorporates implicit or explicit solvent molecules. One of ordinary skill in the art will understand that implicit solvation (also known as continuum solvation) is a method of representing solvent as a continuous medium instead of individual “explicit” solvent molecules most often used in SMD simulations and in other applications of molecular mechanics. In certain embodiments, the steered molecular simulation incorporates water molecules. In certain embodiments, the steered molecular simulation incorporates implicit or explicit water molecules. In certain embodiments, the steered molecular simulation incorporates explicit ion molecules. In certain embodiments, the steered molecular simulation incorporates a lipid bilayer. In certain embodiments, the lipid bilayer incorporates explicit lipid molecules. In certain embodiments, the lipid bilayer incorporates explicit phopholipid molecules. In certain embodiments, the lipid bilayer incorporates a solvated lipid bilayer. In certain embodiments, the lipid bilayer incorporates a hydrated lipid bilayer. In certain embodiments, the hydrated lipid bilayer incorporates explicit phospholipid molecules and explicit water molecules.

In certain embodiments, one or more identified key residues of the hNav1.5 channel's ion permeation pathways are selected from the group consisting of Phe892, Phe934, Phe1418, Phe1459, Phe1465 and Phe1760. In certain embodiments, one or more identified key residues of the hNav1.5 channel's ion permeation pathways are selected from the group consisting of Phe892, Phe934, Phe1418, Phe1459, and Phe1465. In certain embodiments, one or more identified key residues of the hNav1.5 channel's ion permeation pathways are selected from the group consisting of Phe366, Trp374, Phe892, Trp899, Phe1418, Trp1421, Phe1705, and Trp1713. In certain embodiments, one or more identified key residues of the hNav1.5 channel's ion permeation pathways are selected from the group consisting of Phe892, Phe1418 and Trp1713. In certain embodiments, one or more identified key residues of the hNav1.5 channel's ion permeation pathways are selected from the group consisting of Leu938, Asn1463, Asn1472, and Asn1774. In certain embodiments, one or more identified key residues of the hNav1.5 channel's ion permeation pathways are selected from the group consisting of Phe389, Phe402, Tyr403, Tyr416, Asn932, Ser1333, Val1337, Cys1341, Phe1344, and Tyr1767. In certain embodiments, the identified key residues of the hNav1.5 channel's ion permeation pathways is Phe366, Trp374, Phe389, Phe402, Tyr403, Tyr416, Phe892, Trp899, Asn932, Phe934, Leu938, Ser1333, Val1337, Cys1341, Phe1344, Phe1418, Trp1421, Phe1459, Asn1463, Phe1465, Asn1472, Phe1705, Trp1713, Tyr1767, or Asn1774.

In some embodiments, intricate chemical mechanisms are not accessible within the affordable computational limits of classical MD simulations and in such cases, enhanced sampling MD approaches such as SMD simulations can be useful, which was developed to mimic the experimental single-molecule force spectroscopy techniques, such as atomic force microscopy (AFM), bio-membrane force probe and optical and magnetic tweezers, in the simulations. The SMD method has been successfully applied to understand several biological processes, including but not limited to, ligand association/dissociation to proteins and passage of ions from/to the ion channels (Mandavi and Kuyucak, 2015, “Mechanism of Ion Permeation in Mammalian Voltage-Gated Sodium Channels,” PLoS ONE 10: e0133000; Kona et al., 2007, “A gate mechanism indicated in the selectivity filter of the potassium channel KscA,” Theor. Chem.Acc. 117: 1121-1129). However, molecular details on the ion permeation from the central cavity of the Naᵥ1.5 ion channel into the intracellular environment are presently not known in the literature.

5.2.3.6 Calculation of RMSDs

In certain embodiments, the method optionally comprises the step of calculating the root mean square deviation (RMSD) of Ca atoms relative to a reference structure of the ion channel protein. In certain embodiments, calculation of RMSD is performed to observe the overall behavior of the MD or SMD trajectory, prior to identification of dominant conformations of the cardiac ion channel protein using clustering algorithms (see below).

5.2.3.7 Clustering Algorithms

In certain embodiments, the method comprises the steps of using a clustering algorithm to identify dominant conformations of the selected structure around the identified key residues from the SMD simulation, and selecting the dominant conformations identified from the clustering algorithm.

Clustering algorithms are well known by one of ordinary skill in the art (see, e.g., Shao et al., 2007, “Clustering Molecular Dynamics Trajectories: 1. Characterizing the Performance of Different Clustering Algorithms,” J. Chem. Theory & Computation. 3, 231).

In certain embodiments, one dominant conformation is selected. In certain embodiments, more than one dominant conformation is selected. In certain embodiments, 2 dominant conformations are selected. In certain embodiments, 3 dominant conformations are selected. In certain embodiments, 4 dominant conformations are selected. In certain embodiments, 5 dominant conformations are selected. In certain embodiments, 5 or more dominant conformations are selected. In certain embodiments, 10 or more dominant conformations are selected. In certain embodiments, 20 or more dominant conformations are selected. In certain embodiments, 50 or more dominant conformations are selected. In certain embodiments, 100 or more dominant conformations are selected. In certain embodiments, 150 or more dominant conformations are selected. In certain embodiments, 200 or more dominant conformations are selected. In certain embodiments, 250 or more dominant conformations are selected. In certain embodiments, 300 or more dominant conformations are selected.

5.2.3.8 Docking Algorithms

In certain embodiments, the method comprises the step of using a docking algorithm to dock the conformers of the compound to the dominant conformations of the structure of the cardiac ion channel protein determined from the clustering algorithm.

Various docking algorithms are well known to one of ordinary skill in the art. Examples of such algorithms that are readily available include: GLIDE (Friesner et al., 2004 “Glide: A New Approach for Rapid, Accurate Docking and Scoring. 1. Method and Assessment of Docking Accuracy,” J. Med. Chem. 47(7), 1739-49), GOLD (Jones et al., 1995, “Molecular Recognition of Receptor Sites using a Genetic Algorithm with a Description of Desolvation,” J. Mol. Biol., 245, 43), FRED (McGann et al., 2012, “FRED and HYBRID Docking Performance on Standardized Datasets,” Comp. Aid. Mol. Design, 26, 897-906), FlexX (Rarey et al., 1996, “A Fast Flexible Docking Method using an Incremental Construction Algorithm,” J. Mol. Biol., 261, 470), DOCK (Ewing et al., 1997, “Critical Evaluation of Search Algorithms for Automated Molecular Docking and Database Screening,” J. Comput. Chem., 18, 1175-1189), AutoDock (Morris et al., 2009, “Autodock4 and AutoDockTools4: Automated Docking with Selective Receptor Flexiblity,” J. Computational Chemistry, 16, 2785-91), IFREDA (Cavasotto et al., 2004, “Protein Flexibility in Ligand Docking and Virtual Screening to Protein Kinases,” J. Mol. Biol., 337(1), 209-225), and ICM (Abagyan et al., 1994, “ICM-A New Method for Protein Modeling and Design: Application to Docking and Structure Prediction from the Distorted Native Conformation,” J. Comput. Chem., 15, 488-506), among many others.

In certain embodiments, the docking algorithm is DOCK, AutoDock, or GLIDE-SP.

5.2.3.9 Identification of Preferred Binding Conformations

In certain embodiments, the method comprises the step of identifying a plurality of preferred binding conformations for each of the combinations of the selected structure of the cardiac ion channel protein (receptor) and the compound (ligand).

In certain embodiments, a clustering algorithm, as described above, is used to identify the preferred binding conformations for each of the combinations of compound and protein. In certain embodiments, the preferred binding conformations are those which have the largest cluster population and the lowest binding energy. In certain embodiments, the preferred binding conformations are the energetically preferred orientation of the compound (ligand) docked to the dominant conformations of the selected structure around the identified key residues from the SMD simulation, in particular, conformations that represent the closed state of the hNav1.5 channel, to form a stable complex. In certain embodiments, there is only one preferred binding conformation for the docked compound. In other embodiments, a compound has more than one preferred binding conformation of the docked compound with the receptor.

In certain embodiments, a compound that has a binding energy in the preferred binding conformations being equal or less than the binding energy of a known blocker of the cardiac ion channel protein is predicted to be cardiotoxic. In certain embodiments, a compound that has a binding energy in the preferred binding conformations exceeding the binding energy of the known blocker of the cardiac ion channel protein is predicted to have reduced risk of cardiotoxicity or not be cardiotoxic.

In certain embodiments, a compound that has a binding energy in the preferred binding conformations being equal or less than the binding energy of a known blocker of the cardiac ion channel protein is cardiotoxic. In certain embodiments, a compound that has a binding energy in the preferred binding conformations exceeding the binding energy of the known blocker of the cardiac ion channel protein has reduced risk of cardiotoxicity or is not cardiotoxic.

In certain embodiments, a compound that is predicted to be cardiotoxic blocks the channel in the preferred binding conformations. In certain embodiments, a compound that is predicted to have reduced risk or cardiotoxicity or to not be cardiotoxic does not block the channel in preferred binding conformations.

5.2.3.10 Optimizing Preferred Binding Conformations

In certain embodiments, the method comprises the step of optimizing the preferred binding conformations using MD or SMD, as described above. In certain embodiments, the MD or SMD is scalable MD or SMD, respectively. In certain embodiments, the MD or SMD uses NAMD software.

5.2.3.11 Calculation of Binding Energies, ΔG_{calc}

In certain embodiments, the method comprises the step of calculating a binding energy of the compound (ligand) using constrained MD simulations of the preferred binding conformations of the cardiac ion channel protein (receptor). Calculation of binding energies using a combination of molecular mechanics and solvation models are well known by one of ordinary skill in the art (see, e.g., Kollman et al., 2000, “Calculating Structures and Free Energies of Complex Molecules: Combining Molecular Mechanics and Continuum Models,” Acc. Chem. Res. 3B, 889-897).

In certain embodiments, the method further comprises outputting the selected calculated binding energies, ΔG_{calc}, and comparing them to the binding energy of a known blocker of the cardiac ion channel protein. In certain embodiments, the method further comprises the step of providing an IC₅₀ values for each of the combination of the cardiac ion channel protein and the compound using the calculated binding energy of the compound and a function correlating IC50 values and binding energies of compounds known to bind to the cardiac ion channel protein.

In this regard, the IC₅₀ (concentration at which 50% inhibition is observed) values measured from, for example, in vitro biological assays can be converted to the observed free energy change of binding, ΔG_{obs} (cal mol⁻¹) using the relation: ΔG_{calc}=RT lnKᵢ, where R is the gas constant, R=1.987 cal K⁻¹mol⁻¹, T is the absolute temperature, and Kᵢ is approximated to be the IC₅₀ measured for a particular compound, i. Accordingly, ΔG_{calc} may be compared to ΔG_{obs}, and physiologically relevant concentrations (IC₅₀) for each of the combinations of protein and compound.

5.2.3.12 Prediction of Cardiotoxicity and Selection of Compound

In certain embodiments, the method comprises prediction of cardiotoxicity and selection of a compound based on (i) classification of the compound based on comparison to known blockers of the cardiac ion protein channel and/or (ii) calculated binding energies.

5.2.3.12.1 Classification of Compound Based on Comparison to Known Blocker

In certain embodiments, where the calculated binding energy, ΔG_{calc}, of the compound in the preferred binding conformations exceeds the binding energy of the known blocker of the cardiac ion channel protein, the compound is predicted to have reduced risk of cardiotoxicity, thus identifying the compound as a “non-blocker.” Under such circumstances, the “non-blocking” compound is selected for further development or possible use in humans, or to be used as a compound for further drug design. In certain embodiments, further clinical development may comprise further testing for cardiotoxicity with other ion channels, for example, potassium or calcium ion channels, using the methods disclosed herein.

In certain embodiments, wherein the calculated binding energy, ΔG_{calc}, of the compound in the preferred binding conformations is equal or less than the binding energy of a known blocker of the cardiac ion channel protein, the compound is predicted to be cardiotoxic, thus identifying the compound as a “blocker.” Under such circumstances, the “blocking” compound is not selected for further clinical development or for use in humans. However, under such circumstances, the method may further comprise the step of using a molecular modeling algorithm to chemically modify or redesign the compound such that it does not block the ion channel in its preferred binding conformations and retains biological activity to its primary biological target, as described in Section 5.2.3.13 below. As a possible alternative to modification/redesign of the compound, a new compound may also be selected from the collections of a chemical or compound library, for example, a library of new drug candidates generated by organic or medicinal chemists as part of a drug discovery program, as described in Section 5.2.3.14 below.

5.2.3.12.2 Calculated Binding Energies

In certain embodiments, where the calculated binding energies, ΔG_{calc}, for the preferred binding conformations compare to physiologically relevant compound concentrations of greater than or equal to 100 μM, binding affinity is predicted to be weak. Under such circumstances, the compound is predicted to have reduced risk of cardiotoxicity at therapeutically relevant concentrations. The compound may be selected for further development or possible use in humans, or to be used as a compound for further drug design. In certain embodiments, further clinical development may comprise further testing for cardiotoxicity with other ion channels using the methods disclosed herein.

In certain embodiments, where the calculated binding energies, ΔG_{calc}, for the preferred binding conformations compare to physiologically relevant compound concentrations of less than or equal to 1 μM, binding affinity is predicted to be moderate to strong. The compound is predicted to be cardiotoxic at therapeutically relevant concentrations, and the compound is not selected for further clinical development or for use in humans. However, under such circumstances, as described above, the method may further comprise the step of using a molecular modeling algorithm to chemically modify or redesign the compound, or as a possible alternative, selecting a new compound from the collections of a chemical or compound library, as described in the sections below.

5.2.3.13 Modification/Redesign of Compounds

In certain embodiments, the method further comprises the step of using a molecular modeling algorithm to chemically modify or design the compound such that it does not block the cardiac ion protein channel in any of its preferred binding conformations.

In certain embodiments, the method comprises repeating steps g) through j) for the modified or redesigned compound. In certain embodiments, the method comprises repeating steps a) through j) for the modified or redesigned compound.

For example, if a chemical moiety of a compound identified as a “blocker” is found to be responsible for blocking, obstructing, or partially obstructing the hNav1.5 channel, that chemical moiety may be modified in silico using any one of the molecular modeling algorithms disclosed herein or known to one of ordinary skill in the art. The modified compound may then be retested by repeating steps g) through j) of the methods disclosed herein.

Following re-testing, if the modified compound does not block, obstruct, or partially obstruct the ion channel in any of its preferred binding conformations, the modified compound may now be identified as a “non-blocker.” The modified compound may now be characterized as having reduced risk of cardiotoxicity, and selected for further development or possible use in humans, or to be used as a compound for further drug design. By such modification/redesign, potentially cardiotoxic compounds at risk for QT interval prolongation may be salvaged for further clinical development.

In certain embodiments, the modified or redesigned compound does not block the ion channel in its preferred binding conformations, but retains selective binding to a desired biological target, as described in Section 5.2.3.14 below.

5.2.3.14 Selection of New Compound from a Chemical Library

As an alternative to modification/redesign of the compound, a new compound may also be selected from the collections of a chemical or compound library, for example, new drug candidates generated by organic or medicinal chemists as part of a drug discovery program.

For example, once the methods disclosed herein identify a chemical moiety of a original tested compound as a “blocker” that is responsible for blocking, obstructing, or partially obstructing the ion channel, a new compound from a chemical library may be selected wherein, for example, the new compound does not comprise the moiety found to be responsible for the blocking, obstructing, or partially obstructing of the ion channel.

The new compound may then be retested for cardiotoxicity by repeating steps g) through j) of the methods disclosed herein.

Following re-testing, if the new compound does not block, obstruct, or partially obstruct the ion channel in any of its preferred binding conformations, the new compound may be identified as a “non-blocker.” The new compound may be characterized as having reduced risk of cardiotoxicity, and selected for further development or possible use in humans, or to be used as a compound for further drug design. By such selection of a new compound from a chemical library, an entire drug discovery program with potentially cardiotoxic compounds at risk for QT interval prolongation may be salvaged by redirecting the program to safer lead compounds for further clinical development.

The new compound selected from the chemical library may also be tested for selective binding to a desired or primary biological target, which differs from the cardiac ion protein channel, for the modified/redesigned compound.

5.2.4 Biological Aspects

Optionally, the methods disclosed herein include checking in silico predicted cardiotoxicities with the results of an in vitro biological assay, or in vivo in an animal model. The methods disclosed herein may also include validating or confirming the in silico predicted cardiotoxicities with the results of an in vitro biological assay, or with the results of an in vivo study in an animal model.

Accordingly, in certain aspects, provided herein are biological methods for testing, checking, validating or confirming predicted cardiotoxicities.

In certain embodiments, the method comprises testing, checking, validating or confirming the predicted cardiotoxicity of the compound or modified compound using standard assaying techniques which are known to those of ordinary skill in the art.

In certain embodiments, the method comprises testing, checking, validating or confirming the predicted cardiotoxicity of the compound or modified compound in an in vitro biological assay.

In certain embodiments, the in vitro biological assay comprises high throughput screening of ion channel and transporter activities.

In certain embodiments, the in vitro biological assay is a cardiac ion channel protein inhibition assay, for example, the hNav1.5 channel inhibition assay, or electrophysiology measurements in single cells, as explained below.

In certain embodiments, the method comprises testing the cardiotoxicity of the compound or modified compound in vivo in an animal model.

In certain embodiments, the cardiotoxicity of the compound or modified compound is tested in vivo by measuring ECG in a wild type mouse or a transgenic mouse model expressing hNav1.5, as explained below.

5.2.4.1 Sodium Ion Channel Assay

In certain embodiments, the in vitro biological assay is a sodium ion channel assay (e.g., voltage-clamp/patch-clamp assays, ligand binding assays, radiolabeled ion flux assays, 86RB-flux assays, and high-throughput cell-based fluorescent dyes and stably transfected hNav1.5 ion channels), which allows high throughput screening of sodium ion channel and transporter activities.

The assay monitors the permeability of sodium channels to thallium (Tl⁺) ions as described by Hille (Hille, 1972, “The permeability of the sodium channel to metal cations in myelinated nerve,” J. Gen. Physiol. 59(6): 637-58).

The following examples are included to demonstrate preferred embodiments of the disclosure. It should be appreciated by those of ordinary skill in the art that the techniques disclosed in the examples which follow represent techniques discovered by the inventor to function well in the practice of the disclosure, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the disclosure.

### 6. EXAMPLES

Without limiting the scope of the present disclosure, the following examples illustrate the use of steered molecular dynamics (SMD) simulations to identify key residues of the hNaᵥ1.5 channel's ion permeation pathways and calculations of more accurate binding energies of compounds docked to dominant conformations of the channel structure around these identified key residues.

FIGS. 1A and 1B illustrate system block diagrams for selecting a compound that has reduced risk of cardiotoxicity, according to some embodiments of the present invention. Processes illustrated in the system block diagrams (1A) and (1B) are: Target Preparation (includes, e.g., combined de novo/homology protein modeling of hNaᵥ1.5, as exemplified in EXAMPLE 1, below), Ligand Collection Preparation (includes, e.g., translation of the 2D information of the ligand into a 3D representative structure, as exemplified in EXAMPLE 2, below), Ensemble Generation (includes, e.g., Molecular Dynamics simulations and clustering analysis, as exemplified in EXAMPLES 3, 4 and 6, below), Identification of Key Permeation Pathway Residues (includes, e.g., Steered Molecular Dynamics simulations, as exemplified in EXAMPLE 5, below), Docking (includes, e.g., docking and scoring of docked ligand poses, as exemplified in EXAMPLE 7, below), MD Simulations on Top Scoring Poses (includes, e.g., Molecular Dynamics simulations, as exemplified in EXAMPLE 8, below), Rescoring using MM-PBSA (includes, e.g., binding free energy calculation and rescoring of top scoring ligand poses, as exemplified in EXAMPLE 8, below), and Experimental Testing (includes, e.g., a hNaᵥ1.5 channel inhibition assay). The top scoring ligand poses from the Rescoring step can act as positive controls for the next phase screening. The Ensemble Generation, Identification of Key Permeation Pathway Residues, Docking, MD Simulations on Top Scoring Poses, and Rescoring using MM-PBSA steps may be performed on a supercomputer, for example, the “IBM Blue Gene/Q” supercomputer system at the Health Sciences Center for Computational Innovation, University of Rochester (e.g., as shown in the block diagram (FIG. 1B)). The Target Preparation and Ligand Collection Preparation steps may be performed on local machines (e.g., in a Molecular Operating Environment (MOE)).

In certain embodiments, the systems and methods disclosed herein bypass the Target Preparation and Ensemble Generation steps, and use the coordinates of hNaᵥ1.5 models, disclosed herein, as direct input for the Docking step. In certain embodiments, these hNaᵥ1.5 models correspond to the dominant conformations of MD simulations around the identified key residues identified by the SMD simulations, as described herein. In certain embodiments, these coordinates correspond to the closed state of the hNaᵥ1.5 channel. In certain embodiments, these coordinates are selected from the coordinates provided in Table A, disclosed herein.

In certain embodiments, the MD simulations disclosed herein comprise simulations of at least 200,000 atoms and their coordinates (protein, membrane, water and ions). In certain embodiments, the equilibration process of at least 200 ns is equivalent to taking 100 billion steps (10¹¹ steps) updating the position coordinates and velocities of each atom in the system in each of these steps. In certain embodiments, the MD simulations using a current state-of-the art supercomputer, for example, the “IBM Blue Gene/Q” supercomputer system, require an equivalent of 10 million CPU hours which scales approximately linearly with the size of the computational hardware available.

The methods disclosed herein as applied to cardiac ion protein channels, e.g., the hNaᵥ1.5 channel, may be performed as described in EXAMPLES 1-10.

### 6.1 Example 1: Homology Protein Modeling

Homology protein modeling of the α-subunit of the hNaᵥ1.5 channel was performed as follows.

The full-length amino acid sequence (2016 amino acid residues) of the α-subunit of the human Naᵥ1.5 (Uniprot accession code: Q14524-1) was downloaded from the Uniprot database (Magrane et al., 2011, “Uniprot Knowledgebase: A Hub of Integrated Protein Data,” Database 2011). Initially, the full Naᵥ1.5 sequence was dissected into nine sub-domains, four trans-membrane domains (TRM1-TRM4) and five cytoplasmic domains (CYT1-CYT5). Dissection was carried out based on the ProtParam tool (Wilkins et al., 1999, “Protein identification and analysis tools in the ExPASy server,” Methods Mol. Biol. 112: 531-552) on the ExPASy bioinformatics resource portal (Artimo et al., 2012, “ExPASy: SIB Bioinformatics Resource Portal,” Nucleic Acids Res. 40: W597-603). Following dissection, 10 full models for each sub-domains were separately generated using the Iterative Threading Assembly Refinement (I-Tasser) bioinformatics software (Roy et al., 2010, “I-TASSER: a unified platform for automated protein structure and function prediction,” Nat. Protoc. 5: 725-738) based on the NaᵥAB bacterial sodium channel (Payandeh et al., 2012, “Crystal Structure of a Voltage-Gated Sodium Channel in two Potentially Inactivated States,” Nature 486: 135-139) as the main template for the TRM domains. NaᵥAB crystal structures represent the closed-inactivated states of the channel (PDB codes: 3RVY, 3RVZ, 3RWO and 4EKW) (Payandeh et al., 2011). The resolved crystal structures of the two states are very similar with the exception of a very minor shift that is close to the intracellular end of the four S6 helices. These two states of VGSCs are responsible for the binding of common Naᵥ1.5 blockers, including the anti-anginal drug ranolazine (inactivated state) (Sokolov et al., 2013, “Proton-Dependent Inhibition of the Cardiac Sodium Channel Nav1.5 by Ranolazine,” Front Pharmacol 4: 78) and the antiarrhythmic drug mexiletine (closed state) (Undrovinas et al., 2006, Ranolazine Improves Abnormal Repolarization and Contraction in Left Ventricular Myocytes of Dogs with Heart Failure by Inhibiting Late Sodium Current,” J Cardiovasc Electrophysiol, 17 Suppl 1: S169-S177). Generally, common hNaᵥ1.5 blockers bind and stabilize a closed state of the channel. (Payandeh et al., 2012; Sokolov et al., 2013, “Proton-dependent inhibition of the cardiac sodium channel Nav1.5 by ranolazine,” Front. Pharmacol., 4: 78). Thus, only the closed crystal structures were employed for the modeling of TRM domains of hNaᵥ1.5, resulting in a model of a closed conformation of the hNaᵥ1.5 channel.

The amino acid sequences for each sub-domain selected from the main hNaᵥ1.5 sequence is given in Table 2, below.

TABLE 2
The Amino Acid Sequences for the Nine Sub-Domains Dissected
from the Main Naᵥ1.5 Sequence Together with the I-Tasser
Generated TM Scores for the Best I-Tasser Identified Models.

| Name of the | | TM score | |
|---|---|---|---|
| domain/subdomain | Residues | (1-Tasser) | Notes |
| Full hNav1.5 sequence | 1-2016 | — | Uniprot accession |
| | | | code: Q14524-1 |
| CYT1 (N-terminus) | 1-126 | 0.29 | — |
| TRM1 | 127-416 | 0.52 | — |
| CYT2 | 417-709 | 0.43 | Omitted from the |
| | | | final model |
| TRM2 | 710-940 | 0.78 | — |
| CYT3 | 941-1198 | 0.32 | Omitted from the |
| | | | final model |
| TRM3 | 199-1470 | 0.64 | — |
| CYT4 (inactivation gate) | 1471-1523 | 0.50 | — |
| TRM4 | 1524-1772 | 0.68 | — |
| CYTS (C-terminus) | 1773-2016 | 0.46 | — |

A full homology modeling cycle by iterative threading assembly refinement (I-Tasser) started with a multi-threading procedure using the software LOMET followed by alignment of the query protein on the selected templates from the pool of PDB resolved NMR or X-ray crystal structures. Following this extensive threading and alignment procedures, secondary structures of the query protein domain was predicted using the PSIPRED tool. The correctly predicted domains were then assembled and unaligned regions, such as loops, were predicted through ab initio modeling. For each subdomain, top generated models generated by I-Tasser were further refined by the ModRefiner algorithm (Xu et al., 2011, “Improving the physical realism and structural accuracy of protein models by a two-step atomic-level energy minimization,” Biophys. J., 101, 2525-2534). The refined models were checked for potential steric clashes and for proper Ramachandran statistics.

The independent models of the sub-domains were assembled by superimposition over the corresponding segments on the 4EKW crystal structure of the NaVAb using the Smith-Waterman local alignment algorithm (Smith and Waterman, 1981, “Identification of common molecular subsequences,” J. Mol. Biol. 147: 195-197), as implemented in the UCSF Chimera program (Pettersen et al., 2004, “UCSF Chimera—a visualization system for exploratory research and analysis,” J. Comput. Chem. 25: 1605-1612). The four TRM domains were assembled in a clockwise manner such that the four-fold symmetry is retained in the modeled structure. A 90% score for the secondary structure alignment and an iteration threshold of 0.2 Å was employed while assembling the sub-domains over the crystal structure of NaVAb. The cytoplasmic domains were linked to the trans-membrane domains using CHIMERA; three models were generated. These models were further refined through fragment-guided molecular dynamic simulation (or FG-MD) (Zhang et al., 2011, “Atomic-level protein structure refinement using fragment-guided molecular dynamics conformation sampling,” Structure 19: 1784-1795) based refinement. FG-MD aims at refining modeled structures to reach atomic resolution. FG-MD also helps to remove any potential steric clashes through properly guided conformational sampling of the side chains of modeled protein residues. Following the FG-MD refinement, the overall structural quality of these models were assessed by performing Ramachandran analysis using the RAMPAGE webserver and the best model was selected and validated as described below.

Table 2 also shows the I-Tasser calculated TM scores for the best model for each domain and all TRM domains had a high TM score (>0.5) (Zhang et al., 2004, “Scoring Function for Automated Assessment of Protein Structure Template Quality,” Proteins 57: 702-710). The relatively low TM score for TRM1 is believed to be due to the long loop (84 residues, Leu276-Ala359). Before incorporating this loop into the final model, it was first excised and then modeled separately with I-Tasser followed by a structural refinement using a short, all atoms solvated MD simulation (≈1 ns). Finally, the TRM domains were assembled by superposition on the NaᵥAb wild type crystal structure (PDB code: 4EKW) and the final models were again refined with fragment-guided molecular dynamic simulation FG-MD (Zhang et al., 2011, “Atomic-Level Protein Structure Refinement using Fragment-Guided Molecular Dynamics Conformation Sampling,” Structure 19: 1784-1795).

In the case of CYT1 sub-domain, the best structure selected by I-Tasser was subjected to a short explicit solvent MD simulation for 1 ns and was incorporated in the final model of the hNaᵥ1.5 structure. In addition, there are few hNaᵥ1.5 segments that were crystallized already and reported in the literature. (Wang et al., 2012, “Crystal structure of the ternary complex of a NaV C-terminal domain, a fibroblast growth factor homologous factor, and calmodulin,” Structure: 20, 1167-1176; Sarhan et al., 2012, “Crystallographic basis for calcium regulation of sodium channels,” Proc. Natl. Acad. Sci. USA: 109, 3558-3563). These segments were included in the homology model without modifications. One of these segments included residues 1773-1940, which was extracted from the crystal structure of the calmodulin-binding motif of the C terminus of hNaᵥ1.5 (PDB code: 4DCK) with a resolution of 2.2 Å. In the same way, the inactivation gate segment composed of residues 1491-1522 was adapted from the crystal structure of the hNaᵥ1.5 DIII-IV-Ca/CaM complex (PDB code: 4DJC) with an atomic resolution of 1.35 Å. The CYT2 (residue 417-709) and CYT3 (941-1198) sub-domains, lacking good templates for homology modelling, were omitted from the homology model of hNaᵥ1.5, resulting in decreased computational time during subsequent molecular dynamics simulation of the hNaᵥ1.5 model. Thus, the final models of Naᵥ1.5 included 1465 residues that are topologically subdivided into 7 subdomains, 4 transmembrane (TRM1, TRM2, TRM3 and TRM4) sub-domains, and three cytoplasmic domains (CYT1, CYT4 and CYT5).

To achieve the well established four-fold symmetry, the four domains of Naᵥ1.5 were assembled in a clockwise manner based on the resolved NaᵥAb crystal structure. Assembly was carried out by superposing the domains on the 4EKW crystal structure using the Smith-Waterman local alignment (Smith et al., 1981, “Identification of Common Molecular Subsequences,” J. Mol. Biol. 147: 195-197) algorithm with a 90% score for the secondary structure and an iteration threshold of 0.2 Å as implemented in UCSF Chimera (Pettersen et al., 2004, “UCSF Chimera—a Visualization System for Exploratory Research and Analysis,” J. Comput. Chem. 25: 1605-1612). As a final refinement steps and to remove potential severe steric clashes, the system was finally minimized using the protein preparation wizard in Schrodinger was heavy atoms not allowed to move beyond 0.3 Å.

6.1.1 Selection of hNaᵥ1.5 Model

Three full models for the hNaᵥ1.5 channel were assembled from the various sub-domains generated by I-Tasser. These models were inspected for properly reproducing the overall architecture for VGSC's and were further refined by FG-MD (as described above). One of three models (hereinafter, just referred to as the “hNaᵥ1.5 model”) was selected and used in the subsequent Examples based on the following two criteria.

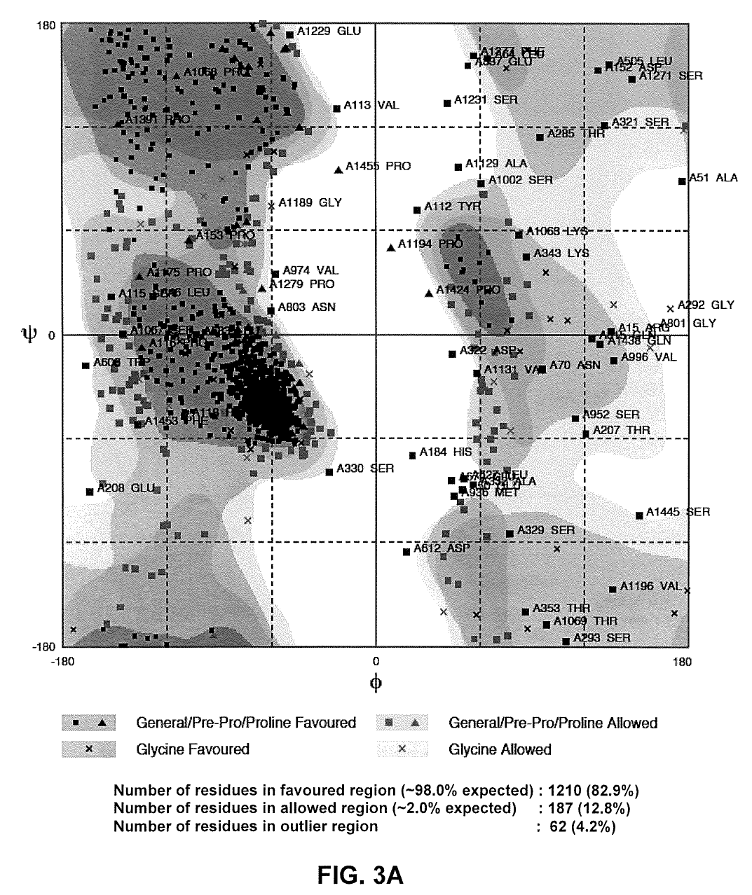

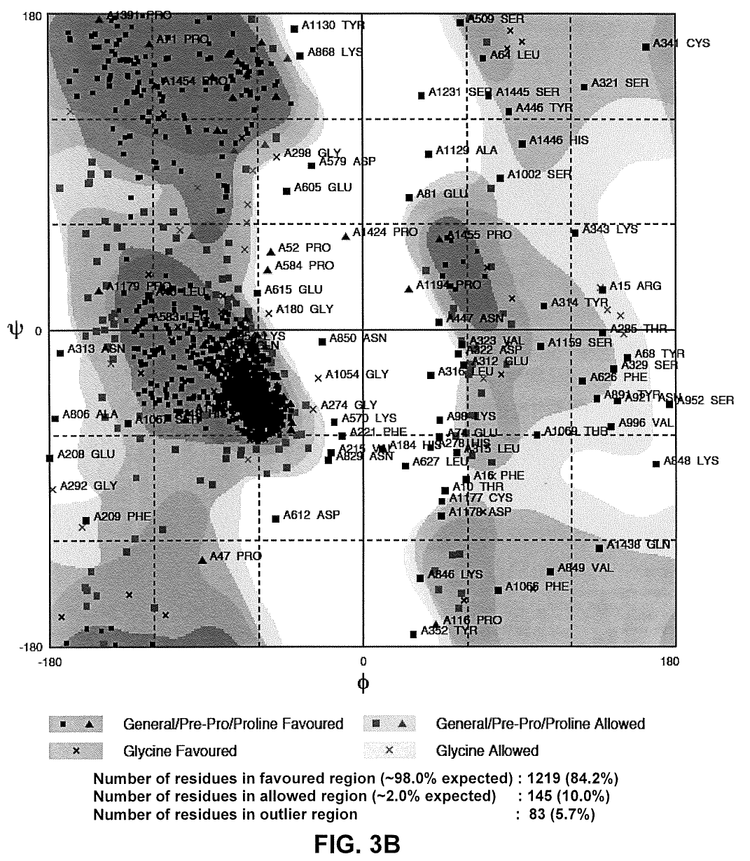

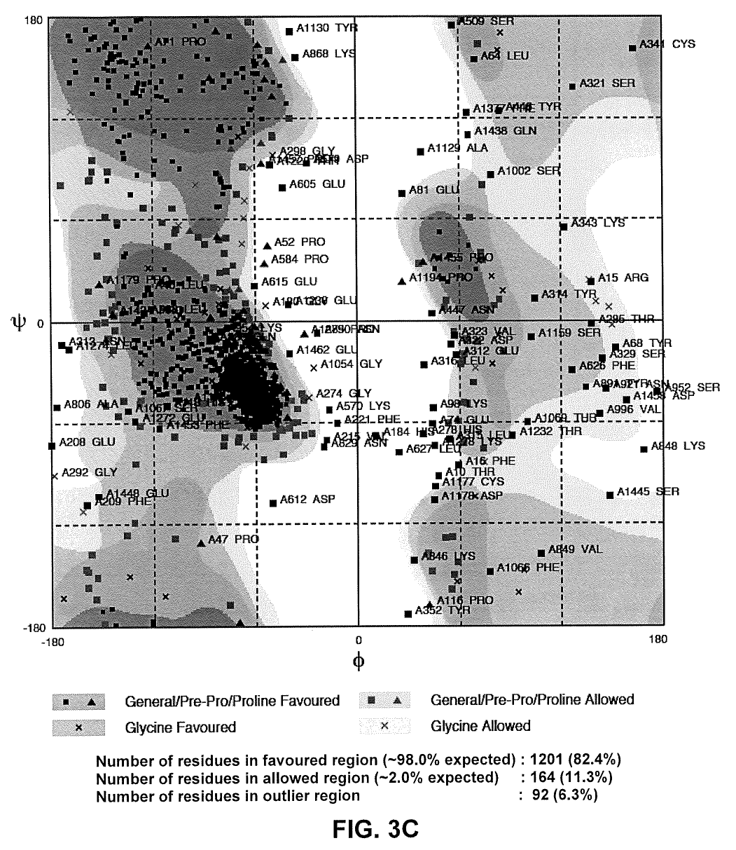

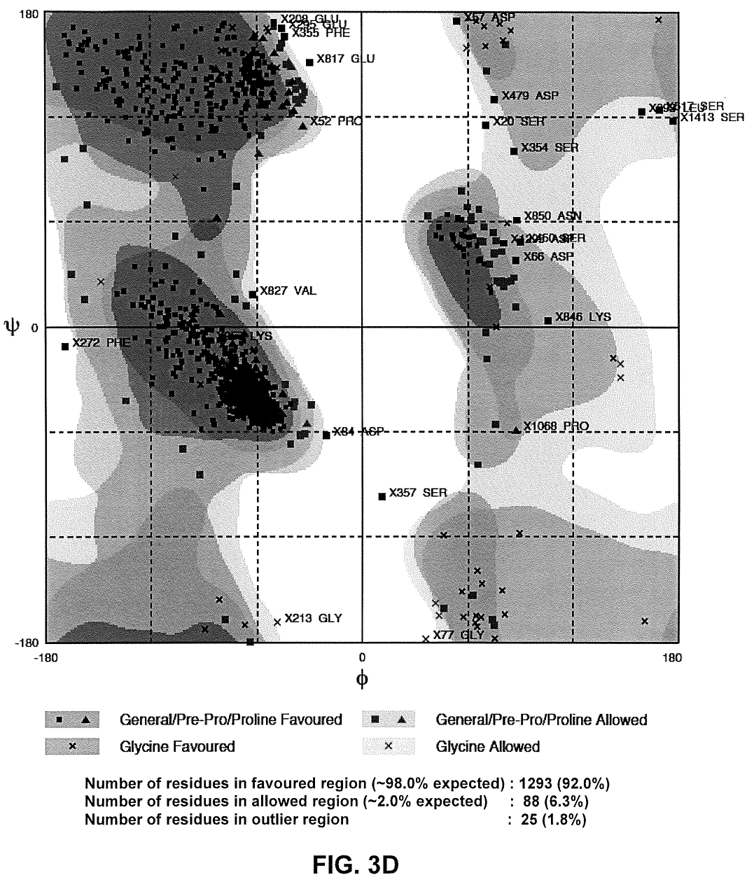

First, the selected model achieved the best Ramachandran plot statistics according to PROCHECK results. FIGS. 3A-3D illustrate the Ramachandran plots for three Naᵥ1.5 models, according to some embodiments of the present invention. The selected model exhibited 87.4% of the residues in the most favored region of the Ramachandran plot and 10.8% in the allowed regions (FIG. 3A). The other two models exhibited approximately 80% of the residues in the most favored region of the Ramachandran plots (FIGS. 3B and 3C).

Second, the selected model displayed excellent conservation of its secondary structure during subsequent MD simulations. By running MD for the three models in parallel their stability and the conservation of the secondary structure was investigated in the trans-membrane helical regions. After approximately 100 ns MD simulation, the non-selected models started to gradually loose the integrity of their helical structures, whereas the selected model exhibited good conservation of the helical structures throughout the simulation (FIG. 3D). The conservation of helical structures is generally used to assess the quality of the generated models and.

FIGS. 4A and 4B illustrate side and top views for a three-dimensional (3D) structure of the hNaᵥ1.5 model, according to some embodiments of the present invention. The figure shows the overall architecture of the channel, comprised of three regions: extracellular, intracellular and trans-membrane (TRM). The Naᵥ1.5 model included four domains (DI, DII, DIII and DIV), which formed the pore region at the middle of the channel as shown in FIG. 4B. Each domain is comprised of two sub-domains, a TRM segment and a cytoplasmic (CYT) sub-domain. The overall structure is assembled as follows: DI comprises CYT1 (N-terminus) and TRM1, DII comprises TRM2, DIII comprises TRM3 and CYT4 (the inactivation gate), and DIV comprises TRM4 and CYT5 (C-terminus). From the intracellular (cytoplasmic) side of the membrane, the trans-membrane sub-domains are connected through the cytoplasmic sub-domains. The four domains are wrapped against the selectivity filter region comprised from the four DEKA sequences that are splayed over the four domains. This DEKA sequence corresponds to the EEEE sequence in the homo-tetrameric bacterial NaᵥAb ion channel template.

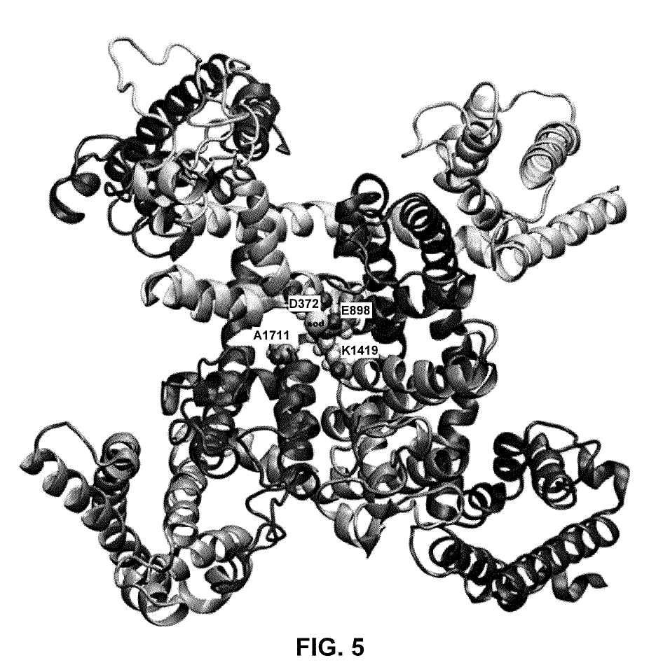

FIG. 5 shows a top view of a 3D structure of the inner region of the Nav1.5 model's selectivity filter (DEKA ring) with a freely sampled sodium ion, according to some embodiments of the present invention. The D372 and E898 residues interact directly with the cation, with E898 interacting with the two oxygens of the carboxylate group and D372 interacts with one oxygen atom from the carboxylate group with the DEKA residues shown in vdW representation. The sodium ion (labelled “sod”) was trapped within the inner selectivity filter in a region of negative potential (as tested and confirmed by a linearized Poisson-Boltzmann algorithm). A rigorous assessment for the Nav1.5 model was its ability to incorporate the selectivity filter residues in the correct place, namely, in the short turn region connecting the P1-P2 helices. In this regard, the assembled domains exhibit the characteristic clockwise arrangement of the four selectivity filter residues splayed over the four domains, Asp372 (DI), Glu898 (DII), Lys1419 (DIII) and Ala1711 (DIV).

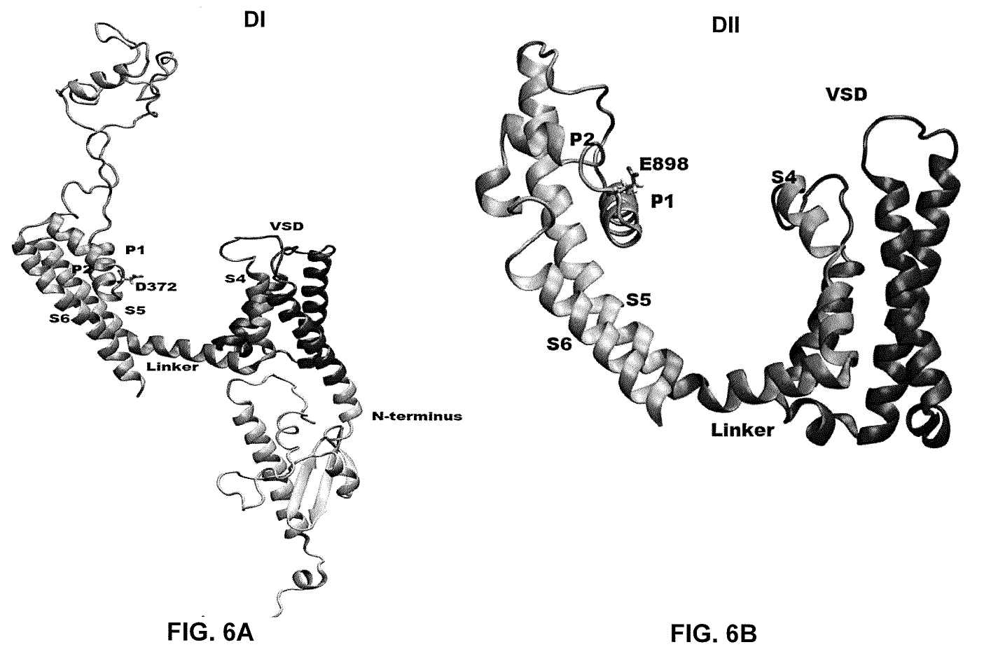

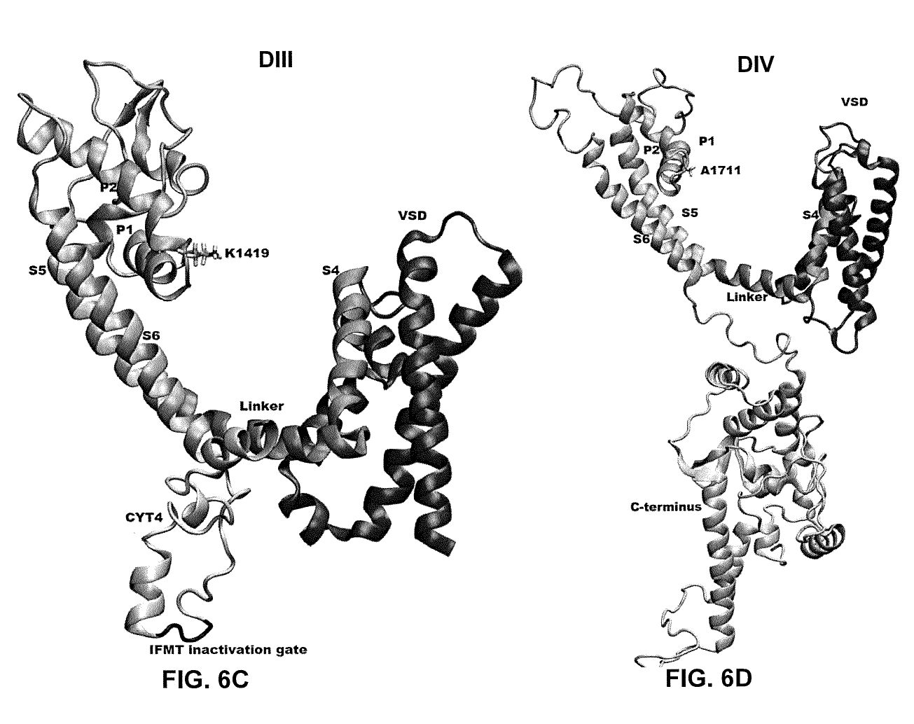

FIGS. 6A-6D illustrate the structures of all four domains (DI, DII, DIII and DIV) of the Nav1.5 model separately, displaying overall similar folding patterns, according to some embodiments of the present invention. Each TRM sub-domain is composed of six long helical segments (namely, S1-S6). The first four-helices (S1-S4) are bundled together in one side to form the voltage-sensing domain (VSD). VSD is an exceptionally powerful nano-scale bio-molecular machinery that responds to changes in the electrical potential across the cell membrane and is conserved among all voltage gated ion channels (Palovcak et al., 2014, “Evolutionary imprint of activation: the design principles of VSDs,” J. Gen. Physiol. 143: 145-156). The S4 segment is a 3₁₀ helix and is characterized by a highly conserved positively charged amino acid residues (LYS and ARG), usually called the “gating charges”. Some of these positively charged residues on S4 are held stable in the trans-membrane region by the salt bridges formed by the negatively charged residues of S1-S3 (ASP and GLU) (Tiwari-Woodruff et al., 2000, “Voltage-dependent structural interactions in the Shaker K(+) channel,” J. Gen. Physiol. 115: 123-138).

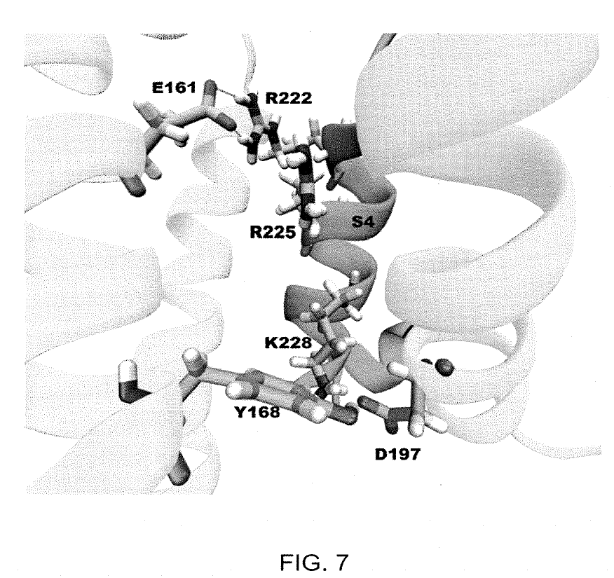

FIG. 7 illustrates a carton representation of the VSDI domain of the hNaᵥ1.5 model, including the S1-S4 helices, R222 forming a salt bridge with E161 and R225 pointing toward the central axis of the VSD (thereby likely effecting the gating current of hNaᵥ1.5), and the lower cluster of interacting residues, Tyr168, Asp179 and Lys225, which form a septum preventing the outward movement of waters and ions through the VSD pore, according to some embodiments of the present invention. FIG. 7 illustrates these interactions of the S4 helix in the VSD of the Naᵥ1.5 model. It is known that a conformational change and an outward movement of S4 helix, in response to the change in the membrane potential, activates the channel to facilitate the passage of the ions through the pore (Catterall, 2014, “Structure and function of voltage-gated sodium channels at atomic resolution,” Exp. Physiol. 99: 35-51). The last two helices, S5 and S6, (S5-S6, FIGS. 6A-6D) are the pore forming segments. The S5 helix is a long segment (of approximately 40 residues in length) that lies parallel to the S4 helix and, through a linker; then extends vertically to the trans-membrane region. A loop then connects S5 with two short helices (P1 and P2), which are named as the pore helices. The S6 segment connects to the P2 helix via a short turn and extends vertically towards the intracellular part of the channel. The short turns connecting P1 and P2 helices in all the domains contain the selectivity specific residues, Asp372 (DI), Glu898 (DII), Lys1419 (DIII) and Ala1711 (DIV) (FIGS. 6A-6D), which together form a filter that selectively captures the sodium ions into the channel.

Mutating any of the residues forming the selectivity filter not only affects the selectivity of the channel, but also the gating kinetics of the channel (Lipkind et al., 2008, “Voltage-gated Na channel selectivity: the role of the conserved domain III lysine residue,” J. Gen. Physiol. 131: 523-529; Hilber et al., 2005, “Selectivity filter residues contribute unequally to pore stabilization in voltage-gated sodium channels,” Biochemistry 44: 13874-13882). Thus, the orientations of these DEKA residues are considered to influence the activity of Naᵥ1.5 structure. As it can be seen in FIG. 4B these residues in the Naᵥ1.5 model are oriented inward of the channel, which is suitable for filtering sodium ions.

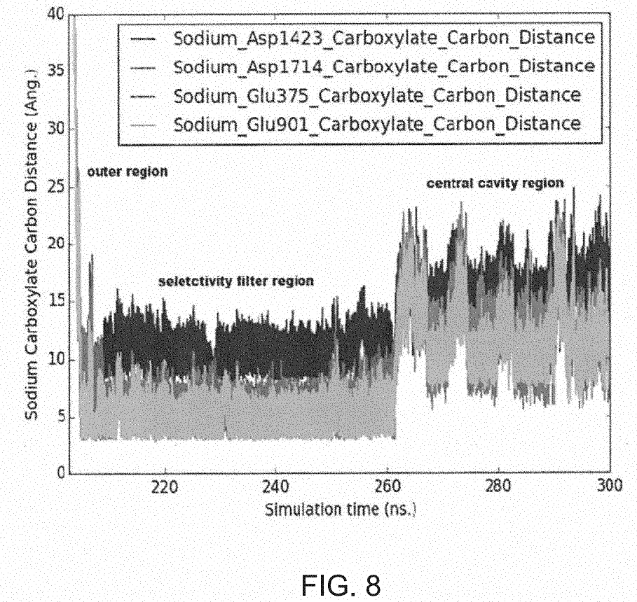

Another layer of the selectivity filter is located above the DEKA ring and is called the “outer selectivity filter”. This filter encompasses four amino acid residues, which are Glu375 (DI), Glu901 (DII), Asp1423 (DIII) and Asp1714 (DIV), which again are spread across the four domains; as listed. With the exception of Asp1423, the carboxylate groups of all other residues in the outer selectivity filter are close to the main axis of the inner selectivity filter. Mutational studies showed that Asp1423 has the least influence on the VGSCs sensitivity for blockade by tetrodotoxin (TTX) in TTX sensitive VGSCs, such as Naᵥ1.4 (Fozzard et al., 2010, “The tetrodotoxin binding site is within the outer vestibule of the sodium channel,” Mar. Drugs 8: 219-234). To explore the orientation of Asp1423 side chain in more details and to examine any potential influence on the entry of sodium, the time-evolution of distances between sodium ion and carboxylate carbon atoms of the outer selectivity filter residues Asp and Glu are given in the supplementary information (FIG. 8). As shown in FIG. 8, after approximately 200 ns, the ion got trapped into the selectivity filter and was later released into the central cavity at about 260 ns. For example, the ion reaches a distance of approximately 5 Å from residues Glu375, Glu901 and Asp1714, whereas it resides at approximately 10 Å from Asp1423.

FIG. 8 illustrates the fluctuations of the atomic distances between sodium ion and the carboxylate carbon atoms of the negatively charged residues (Asp and Glu) in the outer selectivity filters during the 200-300 ns of classical MD simulations. Three of the four outer selectivity filter residues, namely Glu375, Glu901 and Asp1714 first trapped sodium first, with the fourth residue, Asp1423 not interacting with sodium, due to its larger the distance from the sodium ion. The sodium then moved into the inner filter, where it interacted with Asp372 and Glu898.

The coordinates for hNaᵥ1.5 generated from the homology modeling described in EXAMPLE 1, above, are provided in the Table A attached hereto as an appendix is incorporated by reference herein and forms part of the present disclosure. These coordinates were used as input for the MD simulations, described in EXAMPLE 3 below.

### 6.2 Example 2: Compound (Ligand) Preparation

The software MOE (Molecular Operating Environment) from Chemical Computing Group (CCG) (http://www.chemcomp.com/press releases/2010-11-30.htm) was used to translate the 2D information of a compound (ligand) into a 3D representative structure. MOE also generated variants of the same ligand with different tautomeric, stereochemical, and ionization properties. All generated structures were conformationally relaxed using energy minimization protocols included in MOE.

Alternatively, or in addition, the software LigPrep from the Schrödinger package (Schrödinger Release 2013-2: LigPrep, version 2.7, Schrödinger, LLC, New York, N.Y., 2013) may be used to translate the 2D information of a compound (ligand) into a 3D representative structure. LigPrep may also be used to generate variants of the same ligand with different tautomeric, stereochemical, and ionization properties. All generated structures may be conformationally relaxed using energy minimization protocols included in LigPrep.

### 6.3 Example 3: Molecular Dynamics Simulations

Following the modeling of the hNaᵥ1.5 structure, extensive classical molecular dynamics (MD) simulations were performed on the hNaᵥ1.5 model. Initially, the system was prepared using the CHARMM-GUI routine for building membrane proteins (Wu et al., 2014, “CHARMM-GUI Membrane Builder toward realistic biological membrane simulations,” J. Comput. Chem. 35, 1997-2004).

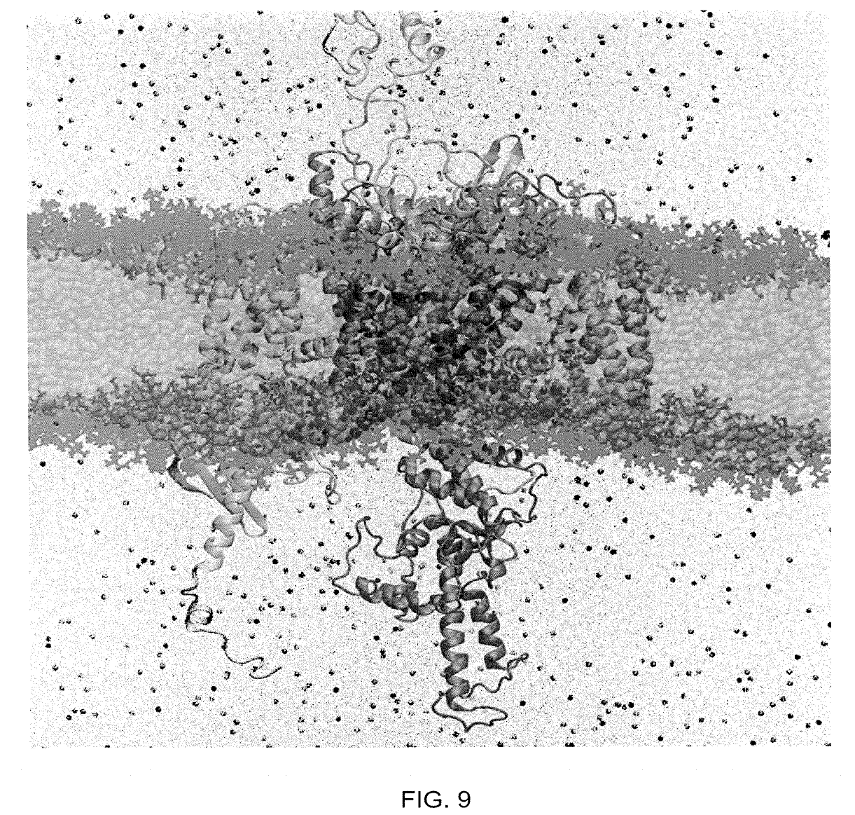

FIG. 9 illustrates a side view of a 3D structure of hNav1.5 channel model in unit cell of the MD simulation with phospholipid bilayer, waters of hydration, and ions, according to some embodiments of the present invention. The atomistic model of the hNaᵥ1.5 protein was sandwiched between two lipid bilayers of 400 l-palmitoyl-2-oleoyl-sn-glycero-3-phosphocholine (POPC) lipids in each layer. These layers were further topped up with a layer of TIP3P water molecules and an ionic concentration of 150 mM NaCl solution, both in the upper (15 Å thickness from the protein) and lower (20 Å thickness from the protein) regions. Thus, a well-packed hNaᵥ1.5 system with a total size of approximately 573,000 atoms was prepared and set-up for MD simulations.

All-atom MD simulations were carried out for the selected models using NAMD (Not (just) Another Molecular Dynamics program) (Phillips et al., 2005, “Scalable Molecular Dynamics with NAMD,” J. Comput. Chem., 26, 1781-1802; Kalé et al., 1999, “NAMD2: Greater Scalability for Parallel Molecular Dynamics,” J. Comp. Phys. 151, 283-312) on a Blue Gene\Q supercomputer. Atomic coordinates were saved to the trajectory every 10 ps. Atomic fluctuation (B-factors) and root mean deviations from the reference structures (RMSD) were calculated, according to the methodologies of EXAMPLE 4 below.

MD simulations were carried out at 300 K, and physiological pH (pH 7.4) and 1 atm using the all-hydrogen AMBER99SB force field for the protein (Homak et al., 2006, “Comparison of Multiple Amber Force Fields and Development of Improved Protein Backbone Parameters,” Proteins 65, 712-725) and the generalized AMBER force field (GAFF) for the ligands (Wang et al., 2004, “Development and Testing of a General Amber Force Field,” J. Comput. Chem. 25, 1157-1174).

The system was then exposed to multiple stages of minimization, heating and equilibration, before starting the production simulations. At the preliminary stage, two minimization phases were carried out to obtain reasonable starting structures. In the first phase, 50,000 steps of energy minimization were carried out, in which only the lipid tails, water molecules and ions were allowed to move freely and the rest of the system were heavily restrained to their original positions. This step ensured the removal of any severe steric clashes that may have resulted from the improper wrapping of the membrane around the protein.

In the next minimization phase, the entire system was constrained with 100 kcal/mol force constant and was energy minimized for 25,000 steps. The heavy constrains on the system were gradually reduced in the three subsequent minimization cycles, from 100 kcal/mol→450 kcal/mol→5 kcal/mol→1 kcal/mol, with each minimization cycle performed for 25,000 steps. Following that, each system was slowly heated to 310 K for 5 ns and using a 1 fs integration time step, with the restrains retained at 1 kcal/mol on the protein backbone. Next, the system was equilibrated with a much-reduced restraint of 0.5 kcal/mol on the protein backbone and in two 10 ns simulations; the first simulation was run with a 1 fs time step and the later with a 2 fs time step.

Additional refinement simulation was then carried out for 100 ns using 0.1 kcal/mol constrains on the Cα carbons of the TRM subdomains. Finally, the production simulation was performed without any constraints and for approximately 580 ns. The Langevin thermostat (Davidchack et al., 2009, “Langevin thermostat for rigid body dynamics,” J. Chem. Phys., 130, 234101) and an anisotropic pressure control were used to keep the temperature at 310 K and the pressure at 1 bar, respectively. A 12 Å cutoff was used to calculate the short-range electrostatic interactions and the Particle Mesh Ewald (PME) summation method was employed to account for the long-range electrostatic interactions. The NBFIX correction term was used for describing the interactions between the sodium ions and the charged carboxylate groups (Yoo et al., 2011, “Improved parametrization of Li+, Na+, K+, and Mg2+ ions for all-atom molecular dynamics simulations of nucleic acid systems,” J. Phys. Chem. Lett., 3, 45-50).

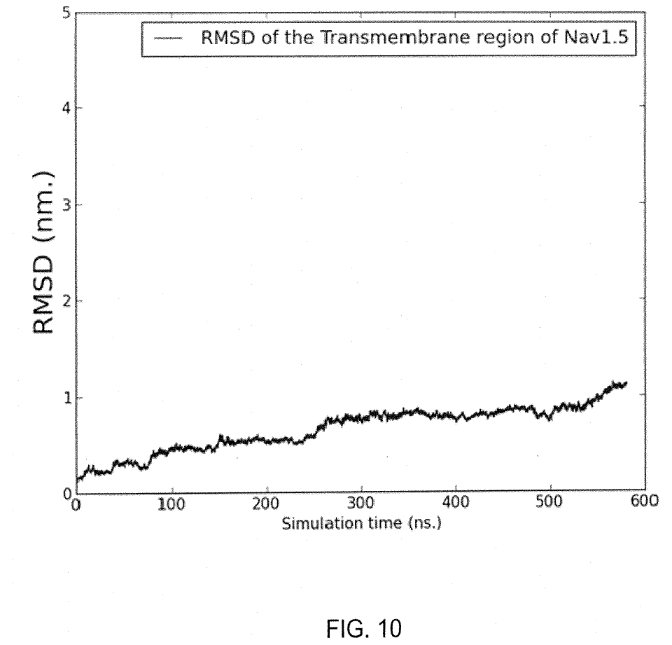

The production MD simulation of the whole system was carried out for approximately 580 ns. FIG. 10 illustrates the stability of the system during MD simulation was initially verified by evaluating the root mean square deviation (RMSD) for the Ca atoms of the trans-membrane region of the Naᵥ1.5 ion channel over the 580 ns MD simulation trajectory, according to some embodiments of the present invention. The systems of over 573,000 atoms showed great stability during the course of the MD simulations as revealed by the Ca fluctuations of the TRM segment being below 1.2 nm. Furthermore, we assessed the stability of the model by calculating the density distribution of the different components of the system across the lipid bilayers. For this purpose, we gathered 2000 snapshots at a regular interval from the last 50 ns of the MD trajectory and averaged them to produce density profiles of various counterparts, such as ions, water, lipid heads and lipid tails.

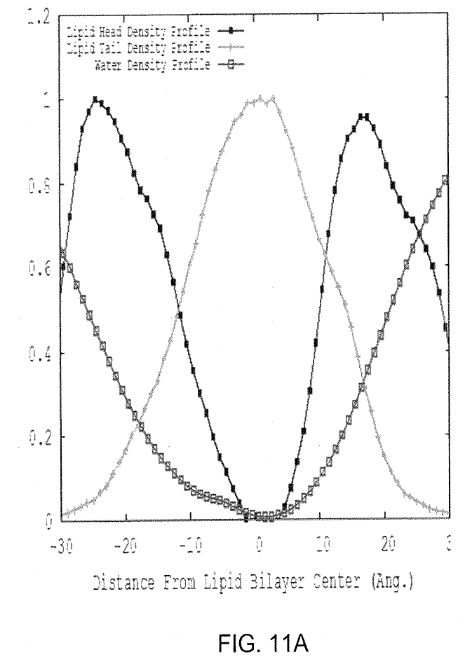

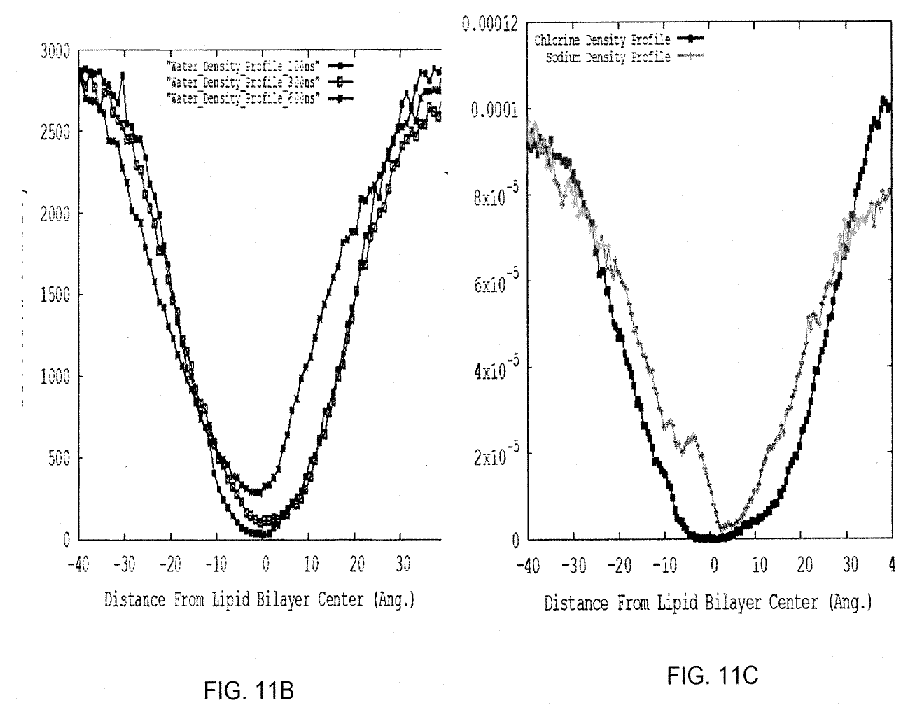

The density profiles of water molecules, lipid heads and lipid tails across the membrane (presented in FIG. 11A) show a uniform distribution across the membrane bilayer normal. The distribution of water molecules across the membrane as a function of the simulation time (shown in FIG. 11B) was also investigated in order to capture any significant reorganization that might have occurred during the MD simulations. Such details could shed some light on the role of solvation of the ion by water molecules during the passage through the ion channel. The profiles in FIGS. 11A and 11C was calculated by averaging over approximately 2000 snapshots of system coordinates gathered uniformly over the last 50 ns of the simulation. The distribution of water molecules across the membrane after 100 ns, 300 ns and the last snapshot was analyzed (shown in FIG. 11B). The distribution of water molecules displayed considerable changes, with an increased density of water molecules observed close to the center of the lipid membrane bilayer, as the simulation proceeded. By water molecules potentially penetrating through the selectivity filter along with the sodium ion, water underwent a noticeable reorganization as the simulation proceeded with a higher density close to the membrane center.

FIG. 11C displays the distribution of sodium and chloride ions around the membrane normal. The sodium ions exhibited a higher density than chloride ions in the region close to the center of the membrane, which reflects the selectivity towards sodium ions by the ion channel. Particularly, the density of sodium ions was significantly higher than that of chloride ion, which was almost zero, in the region close to the center, indicating the selective trapping of the sodium ions in the central cavity.

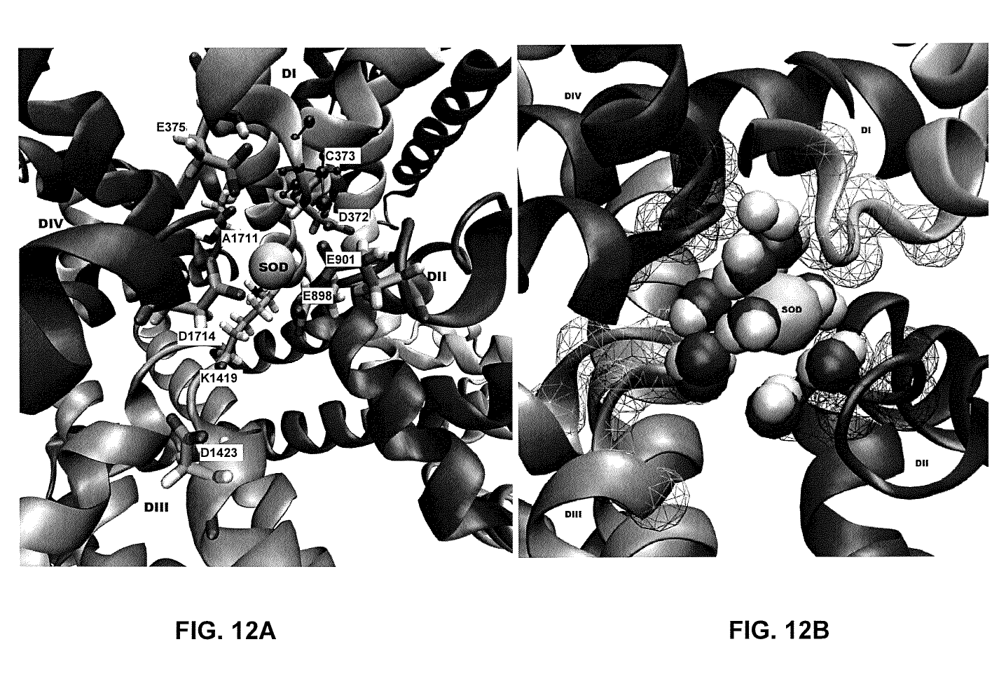

For example, a sodium ion was trapped in the selectivity filter at about 200 ns of the classical MD simulations. After being trapped within the selectivity filters, the sodium ion spent approximately 60 ns binding to the negatively charged residues at the selectivity filter vestibule. These negatively charged residues are found to be responsible for creating a deep electrostatic potential well that can selectively capture the sodium ions. The residues forming the outer selectivity filter, E375 (DI), E901 (DII), D1423 (DIII) and D1714, initially captured the sodium ion, with the ion later being translocated to Asp372 and Glu898 at the inner selectivity filter. FIG. 12A illustrates a top view of Naᵥ1.5 model, in which a sodium ion was trapped within the selectivity filter residues, displaying the residues of the two selectivity filter rings, the inner DEKA (D372, E898, K1419 and A1711) ring and the outer EEDD (E375, E901, D1423 and D1714) ring, are shown as stick representations.

A combination of positively charged, negatively charged and neutral amino acid residues in the selectivity filters were found to be significant for preferentially filtering sodium ions over other ions that are abundant in the physiological microenvironment. For example, a previous mutational study by Favre et al. showed the selectivity towards sodium (over potassium) was reduced by a factor of three when the Glu was mutated to Ala residue (Favre et al., 1996, “On the structural basis for ionic selectivity among Na+, K+, and Ca2+ in the voltage-gated sodium channel,” Biophys. J. 71, 3110-3125). Another study by Sun et al. demonstrated that the sodium/potassium selectivity is almost lost upon mutating the Lys residue of the third domain to Ala, such that the “DEKA” is mutated into “DEAA” (Sun et al., 1997, “On the structural basis for size-selective permeation of organic cations through the voltage-gated sodium channel. Effect of alanine mutations at the DEKA locus on selectivity, inhibition by Ca2+ and H+, and molecular sieving,” J. Gen. Physiol. 110: 693-715). Here, although Lys did not form a barrier to the entry of the sodium ion to the central cavity, Lys acted as a gatekeeper to prevent sodium from returning back to the reverse direction after it passed to the central cavity. In some cases, Lys may form a barrier for the entry of the bulkier potassium ions through the selectivity filter. After the ion moved into the central cavity, it did not move out of the channel, but stayed there until the end of classical MD simulations.

The MD simulations further demonstrate that, unlike potassium channels that dehydrate the ion before passage (Nimigean and Allen, 2011, “Origins of ion selectivity in potassium channels from the perspective of channel block,” J. Gen. Physiol. 137: 405-413), sodium was completely hydrated while entering into the Naᵥ1.5 channel (FIG. 12B). FIG. 12B shows a snapshot of the MD simulation, in which the sodium ion (solid sphere) was captured in the selectivity filter of the hNaᵥ1.5 model and solvated by water molecules (shown as vdW spheres). Thus, the solvation of sodium ion by the water molecules played a role in driving the ion into the ion channel. Previous crystallization experiments demonstrated that the sodium ion is fully hydrated inside the selectivity filter of the NaᵥAb bacterial channel (Payandeh et al., 2011). A possible role of these hydrating water molecules is to replace sodium for binding with the selectivity filter residues, which is supported by the MD simulation where water molecules engaged with Asp and Glu residues allowing the sodium to slide and escape toward the central cavity of the channel.

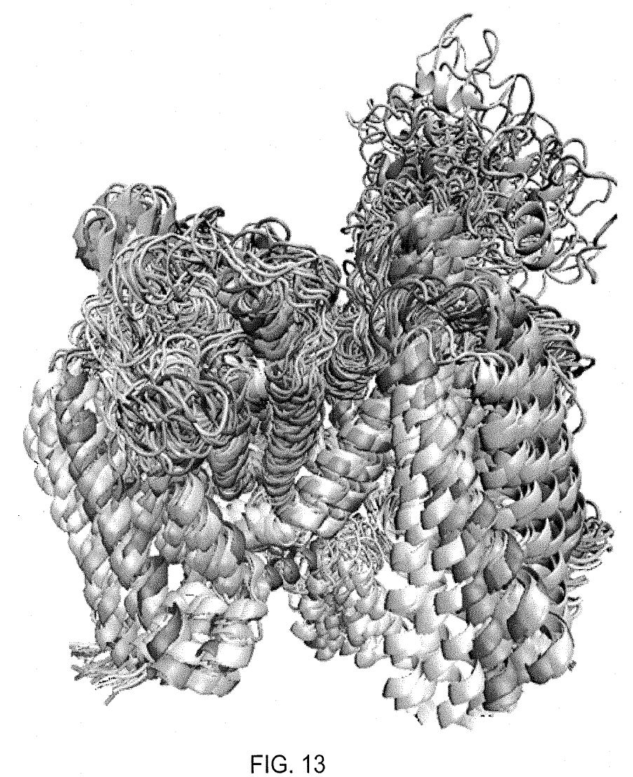

According to the methodologies of EXAMPLE 6 below, iterative clustering of the MD trajectory was then performed to extract dominant conformations of Naᵥ1.5 channel around key residues of the channel's ion permeation pathways identified in EXAMPLE 5. Using these methodologies, twenty (20) dominant conformations for the hNaᵥ1.5 channel were identified, selected through the RMSD coordinates clustering of the MD trajectory around these key permeation pathway residues, as illustrated in FIG. 13.

### 6.4 Example 4: RMSD Calculation

The root mean square deviation (RMSD) of C_{α} atoms relative to a reference structure were calculated as follows:

$$RMSD ( t ) = {[ \frac{1}{{N}^{2}} {\sum }_{i , j} {\vert {r}_{ij} ( t ) - {r}_{ij}^{ref} \vert}^{2} ]}^{1 / 2} , \qquad ( 2 )$$

where N is the number of atoms, and r^{ref} is a reference structure, and is presented in FIG. 10.

Each point in this graph represents a different set of coordinates for the hNaᵥ1.5 model. The separation between two points in the y-axis represents a deviation between the corresponding protein structures. As shown in FIG. 10, the hNaᵥ1.5 channel reached equilibrium almost after 20 ns of simulation where the RMSD points fluctuated around 0.3 Å, while reaching a prolonged equilibrium at 250 ns with an RMSD of about 0.9 Å. By observing the overall behavior of the hNaᵥ1.5 trajectory one can notice the tremendous amount of dynamical transitions of the channel, which can be attributed to the rearrangements of the flexible loops within the protein structure. This allowed the hNaᵥ1.5 structure to explore a wide conformational space, allowing for introducing protein flexibility within the docking procedure as described below.

### 6.5 Example 5: Steered Molecular Dynamics Simulations

Non-equilibrium steered molecular dynamics (SMD) simulations were initially performed on the final snapshot (referred to as “snap1”) from the MD trajectory of EXAMPLE 3. SMD simulations allowed studying the mechanisms and permeation pathways by which a sodium ion is released from the central cavity of the hNaᵥ1.5 channel, and thus identifying key residues of the hNaᵥ1.5 channel's ion permeation pathways. All the parameters and preparation for the SMD simulations were identical to those employed in the classical MD production runs described in EXAMPLE 3, except for the introduction of an artificial force in SMD to pull the ion off the cavity. In the SMD simulations, a dummy atom was attached to the center of mass of the sodium ion using a virtual spring (with a spring constant of k) that is subsequently pulled with a constant velocity v and in a selected direction n. The numerical value of the pulling force at a given time F (t) can be estimated by the above equation (1). All-atom SMD simulations were carried out for the selected models using the NAMD2.9 package (Phillips et al., 2005, “Scalable Molecular Dynamics with NAMD,” J. Comput. Chem., 26, 1781-1802; Kalé et al., 1999, “NAMD2: Greater Scalability for Parallel Molecular Dynamics,” J. Comp. Phys. 151, 283-312) on Blue Gene\Q supercomputer clusters.

In this example, the artificial force was applied on the ion in order to pull the ion out of the central cavity, in the direction \vec{n} of the four S6 helices of the domains (DI-DIV) into the bulk-water (where the intra-cellular environment is present in vivo). To select the optimal values for the constant velocity v and the spring constant k, several trial simulations were carried out with the following combinations force constants k and constant velocities v: (1) k=1 kcal/mol, 2 kcal/mol, 3 kcal/mol, 4 kcal/mol, and 5 kcal/mol, and (2) v=0.25 Å/ps, 0.3 Å/ps, 0.35 Å/ps, 0.4 Å/ps, 0.45 Å/ps, 0.5 Å/ps. From the resulting thirty different simulations, k=4 kcal/mol and 5 kcal/mol in combination with v=0.45 Å/ps were identified as preferred for any subsequent SMD simulations, since these parameter values did not cause significant structural changes in the system due to the external force on a time scale of 1-10 ps.

In addition to snap1, six additional snapshots (referred to as “snap2,” “snap3,” “snap4,” “snap5,” “snap6” and “snap7”) were used for starting points of the SMD simulations. These snapshots were collected from the last 350 ns of the classical MD trajectory at regular intervals of 50 ns per snapshot. The SMD simulations were then performed on the seven different structures of the hNaᵥ1.5 model to investigate the effects of time-dependent conformational changes in the structure of the hNaᵥ1.5 channel on the permeation of the sodium ion from the central cavity to the bulk water. For every structure (snapshot), ten SMD simulations (5 repeats for k=4 kcal/mol and 5 kcal/mol each, and v=0.45 Å/ps) were carried out for a simulation time of 200 ps to test reproducibility of the force profiles generated by the SMD simulations. A total of seventy SMD trajectories were generated and analyzed to develop mechanistic insights into ion permeation from the central cavity of a closed state for the hNaᵥ1.5 channel.

In EXAMPLE 3, a sodium ion, which was captured by the selectivity filter during the early stages of the classical MD simulations, entered into the central cavity of the Naᵥ1.5 channel and remained bound in this area for more than 350 ns timescale. A previous microseconds scale MD study on the bacterial NaᵥAB also found that the sodium ion that passed through the selectivity filter continued to be trapped in the cavity region, suggesting that this cavity in the sodium ion channels may be a binding site for sodium ions (Chakrabarti et al., 2013, “Catalysis of Na+ permeation in the bacterial sodium channel NaVAb” Proc. Nat. Acad. Sci. USA 110: 11331-11336). By employing non-equilibrium SMD simulations and thereby introducing an external force, the trapped ion was forced from the central cavity into the bulk water through the S6 helices of the four domains (DI-DIV) that form the walls of the transmembrane channel. By means of the SMD simulations, the key amino acid residues were identified that control the access to the ion-release pathways, thus providing mechanistic insights into the ion permeation into the cell.

The dissociation processes of the sodium ion and the corresponding force profiles were almost similar in all the ten simulations conducted for each structure, irrespective of the chosen force constant (4 kcal/mol or 5 kcal/mol), providing support that the generated results were mostly free from artifacts due to the initial conditions of the SMD simulations. Furthermore, the ions did not permeate through the same pathways in all the structures, but rather a first and a second permeation pathway, which indicates that the conformational changes occurred in the Naᵥ1.5 structure during the classical MD simulations had impacted the process of ion permeation in SMD simulations.

6.5.1 Ion Permeation Via the First Permeation Pathway (“Pathway 1”)

SMD simulations of the snap1 structure (or the final snapshot) of the MD simulation of EXAMPLE 3 indicated that the sodium ion, under the influence of an extra force, permeated into the intracellular solution (or bulk water) via the S6 helices of domains DII and DIII (hereinafter referred to as “pathway 1”).

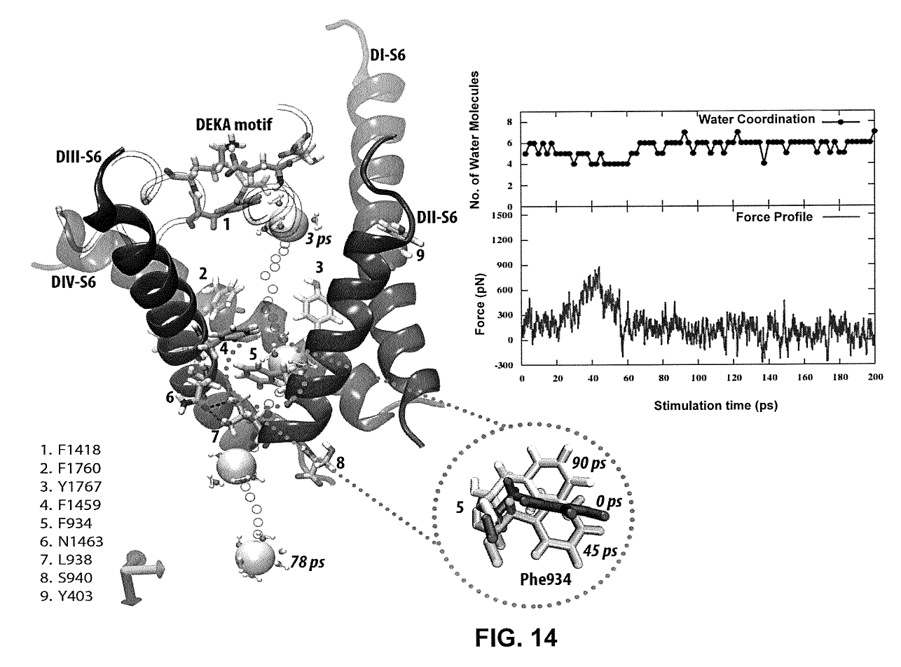

FIG. 14 presents the process of ion permeation from the cavity into the bulk water through pathway 1 and the corresponding force profile of the pulling for this trajectory (of snap1). The four S6 helices of various domains are represented as blue ribbons and the key residues involved in the ion permeation through pathway 1 are shown as stick models and their residue numbers are also listed in the figure. The positions of the sodium ion and the surrounding water molecules (i.e., within 3.5 Å of the ion) at different time durations (3 ps, 42 ps, 63 ps and 78 ps) on the course of SMD simulations are displayed as VDW spheres in yellow (as in the VMD program), while the movements of sodium ion at each step, until it left the S6 helices (or transmembrane segment), which are shown in the background as smaller bubble spheres (FIG. 14). Initially, the Na+ ion was trapped near the DEKA motif at the central cavity of the ion channel before exiting through the S6 helices (shown as ribbons) of the domains DII and DIII along pathway 1. The ion, under the influence of external force, passed through a number of hydrophobic residues (shown as sticks) and was finally released by the conformational changes of F934 (see within the circle) and the disruption of H-bonds between N1463 and L938 residues (shown as broken lines in black). The positions of the ion was surrounded by water molecules at different times (i.e., 3 ps, 42 ps, 63 ps and 78 ps) during the SMD simulation (shown as VDW spheres with the positions of the ion along each step until the release indicated by the smaller CPK spheres).

In this example, the sodium ion was bound close to the DEKA filter and between the P1/P2 turns of DIII (left) and DII (right) domains on either sides, illustrated as tube representations in FIG. 14. As the ion was pulled by the external force, it moved from its initial position when applying a force of approximately 500 pN and passed through the hydrophobic contacts of Phe1760 and Tyr1767 (located on the DIV-S6 helix) that protruded towards the cavity region. At about 20 ps, the forward movement of the sodium ion in this direction was found to be severely obstructed by: (1) the stacking arrangements of two Phe residues, Phe1459 (from DIII-S6 helix) and Phe934 (from DII-S6), and (2) followed by the strong hydrogen bonds (H-bonds) formed between the side chains of Asn1463 (from DIII-S6) and Leu938 (from DII-S6). As a result, the external force increased to approximately 900 pN to rupture these interactions, pulling the ion outside of these barriers.

FIG. 14 further illustrates the force profile for the pulling the ion and the numbers of water molecules surrounding the ion along the trajectory of pathway 1. The force profile of FIG. 14 illustrates a force of approximately 200 pN at 20 ps that gradually increased and reached a peak of approximately 900 pN at 45 ps. At this point, the Phe934 residue flipped its side-chain orientation (FIG. 14) to clear the route for ion permeation. Subsequently, the H-bonds between Asn1463 and Leu938 were also ruptured at a force of approximately 600 pN to release the ion from the cavity barricaded by the S6 helices.

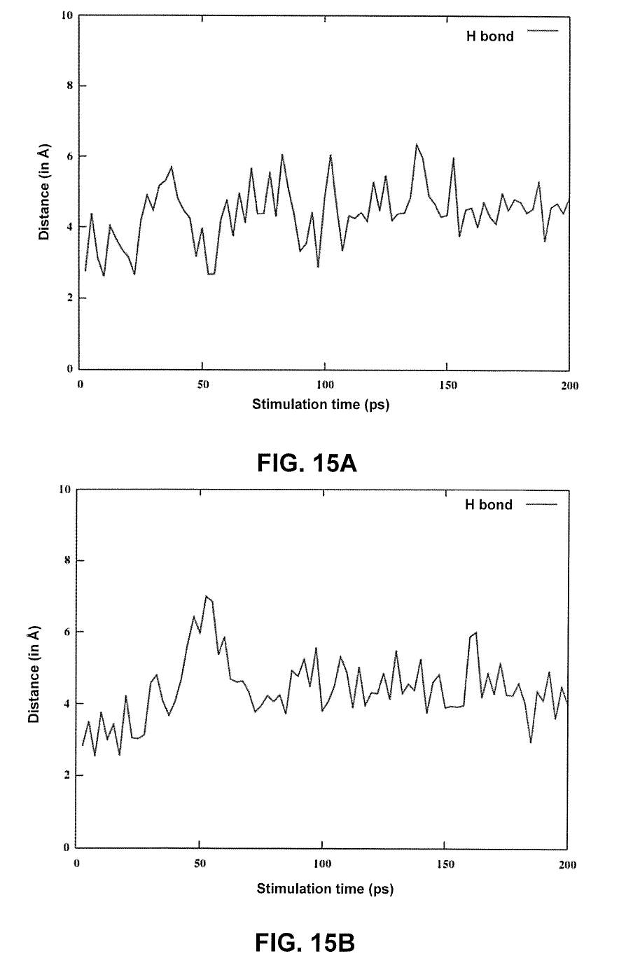

FIGS. 15A and 15B illustrates the time-evolution of H-bond distances between the side chain oxygen atom of Asn1463 and the two hydrogen atoms of the side chain methyl groups in Leu938, which describes that the H-bond reached maximum fluctuations of 5-6 Å during 35-55 ps of SMD trajectory, coinciding with the time frame of the maxima peak in the force profile (FIG. 14).

After completely releasing the ion from the influence of S6 helices (i.e., after 63 ps), the force profile mostly remained as a valley until the end of SMD simulation (200 ps). As shown in FIG. 14, the sodium ion continue to be hydrated by water molecules throughout the SMD trajectory, while the coordination number of water molecules with sodium dropped to four water molecules at the force peak, demonstrating that the ion had to drop water molecules from its solvation shell in order to permeate through the narrow-edge of S6 helices of DII and DIII that was secured by the Phe stacks and H-bond interactions. The coordination number returned to the initial value of about six to seven water molecules after the ion was released into the intra-cellular solution (i.e., bulk water).

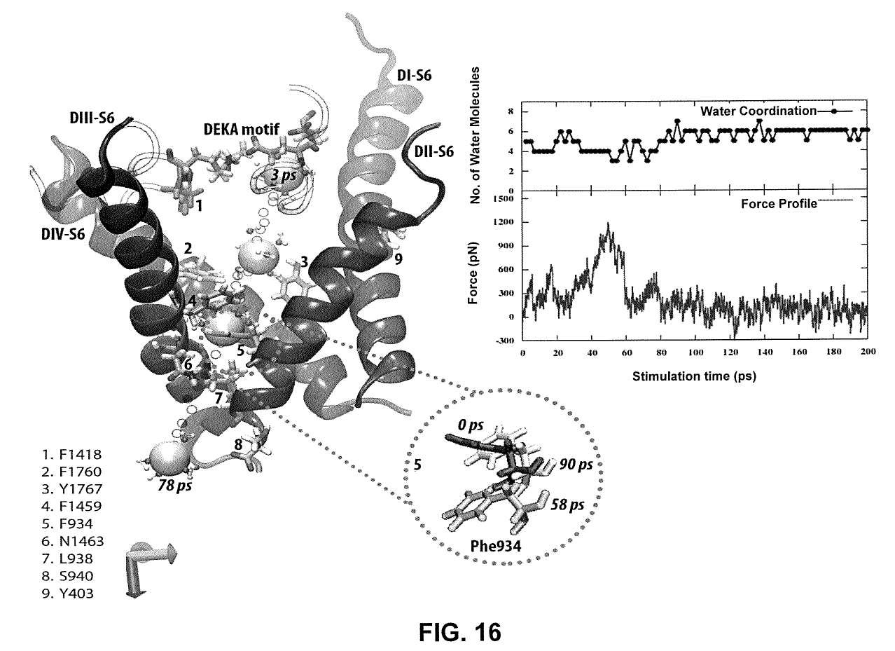

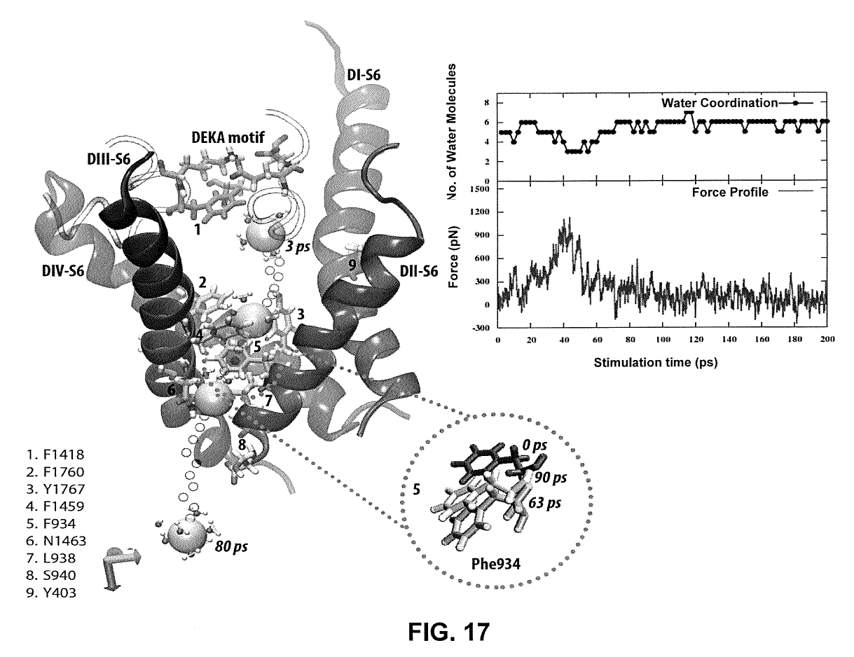

As illustrated in FIGS. 16 and 17, SMD simulations of the snap2 and snap 4 structures found that the sodium ion was also trapped at the central cavity in two other structures, before being released via pathway 1 according to similar mechanisms of permeation found in the SMD simulation of the snap1 structure. Nevertheless, the orientations of P1/P2 turns of DIII and P1 helix of DII based on the snap2 and snap4 structures were significantly different when compared to those using the snap1 starting structure. Especially, for the snap2 SMD simulation, the P1 helix of DII moved significantly towards the left-side, such that some of the residues in this helix (Phe892 and Cys896, for example) were oriented towards the central cavity, thereby constricting the space for the forward movement of the sodium ion in the direction of pathway 1. Hence, the force profile for snap2 SMD simulation displayed two short peaks reaching approximately 600 pN at less than 20 ps of simulation time, which corresponded to breaking of the interactions of sodium ion with the DEKA residues, moving across the P1 helix of DII and passing through the hydrophobic contacts of Phe1760 and Tyr1767. Subsequently, and similar to the snap1 SMD simulation, the force gradually increased to approximately 1200 pN between 25-60 ps, which resulted in breaking the stacking of Phe1459 and Phe934 and Asn1463-Leu938 H-bond interactions. In summary, the ion, under the influence of external force, passed through a number of hydrophobic residues (shown as sticks FIGS. 16 and 17) and was finally released by the conformational changes of Phe934 and the disruption of H-bonds between Asn1463 and Leu938 residues (shown as broken lines).

The number of water molecules coordinating with sodium ion (in its first solvation shell) dropped intermittently while breaking through these barriers, which later picked up and was stabilized between 6-7 water molecules. The ion was released completely from the influence of transmembrane at about 80 ps, after which the force profile remained flat until 200 ps. Similar processes were also found in the ion permeation of the snap4 SMD simulation (FIG. 17). Thus, the ion release along the pathway 1 (between S6 helices of DII and DIII) was controlled by four residues, Phe1459, Phe934, Asn1463 and Leu938, which acted as gatekeepers of the permeation tunnel.

6.5.2 Ion Permeation Via the Second Permeation Pathway (“Pathway 2”)

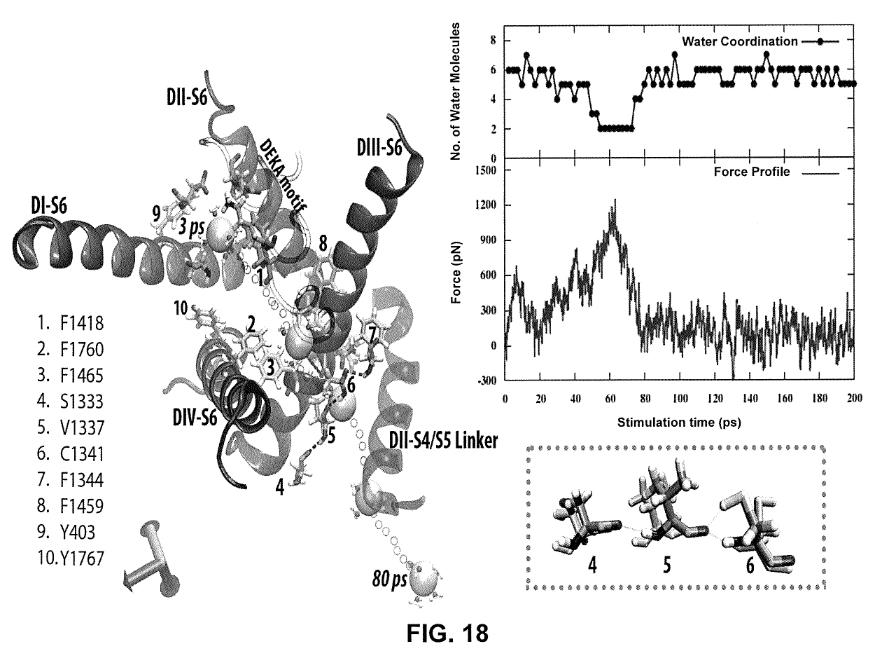

The ion permeation based on the snap3, snap6 and snap7 structure of the MD simulation in EXAMPLE 3 occurred through the S6 helices of domains DIII and DIV (hereinafter referred to as “pathway 2”). The change in the direction of permeation was likely triggered by the orientations of Phe1419 and Phe892 located on the P1/P2 turn of DIII and P1 helix of DII, respectively. FIGS. 18-20 illustrates the SMD simulations based on the snap3, snap6, and snap 7 structures, respectively. The positions of the ion surrounded by water molecules are illustrated at different simulation times as VDW spheres. FIGS. 18-20 further illustrates the ion positions along each step until the exit shown as smaller CPK spheres and the force profiles for the pulling of the ion and the numbers of water molecules surrounding the ions along the pathway 2.

FIG. 18 illustrates the mechanisms of ion expulsion from the cavity based on the snap6 structure of the Naᵥ1.5 model through pathway 2, along with the related force profile of the pulling for this trajectory. At the initial stage, the sodium ion was bound near to the DEKA filter and the P1/P2 turn of DIII and P1 helix of DII moved significantly, so that Phe1418 of domain DIII and Phe892 of domain DII tilted their conformations and blocked the route towards pathway 1. As a result, the ion explored an alternate direction to exit the Naᵥ1.5 channel. The permeation along pathway 2 started by releasing the ion from its initial contacts from the residues forming the DEKA filter, upon which the ion within 1-2 ps encountered a transient cation-π scenario with Phe1418 in its new orientation as compared to permeation along pathway 1. Breaking free the ion from this cation-π interaction then required a considerably higher force of approximately 620 pN at the early stage of SMD simulation, i.e., less than 20 ps. Subsequently, the ion passed through the hydrophobic contacts of Phe1760 and Tyr1767 (located on the DIV-S6 helix) that were facing the region of ion permeation, which resulted in a second force peak of approximately 580 pN as shown in the force profile at a simulation time between 20-40 ps. At about 35 ps of SMD simulation, the sodium ion was located near the surface between DIII and DIV helices, where the onward path was inaccessible due to the stacking of another pair of Phe residues Phe1760 and Phe1465 (similar to pathway 1). Increasing the force caused by this blockade to approximately 900 pN at about 40 ps resulted a tilt of Phe1465 which was not sufficient enough for the ion to move further past Phe1465. A subsequent force increase to approximately 1230 pN resulted in disturbing a chain of H-bond interactions between Ser1333-Val1337-Cys1341-Phe1344. Particularly, the SH group of Cys1341, which formed a strong H-bond with the carbonyl oxygen atom of Val1337, tilted its conformation by approximately 180° thereby rupturing the H-bond with the Val1337 residue, and allowing the ion move further towards the bulk water. This cleared a narrow route for the sodium ion to pass the H-bond fence (at nearly 60 ps) and permeate out of the S6 helices. Following expulsion from S6 helices and permeating via pathway 2, the ion was unable to freely migrate into the bulk water, since it faced the S4/S6 linker of DII in this direction that controlled further ion movement. As a consequence, the force stayed at 500-600 pN while forcing the release of the ion from the influence of this short helical linker. Thus, the force in force profile did not flatten until a simulation time of 80 ps, after which the ion permeated into the bulk water.

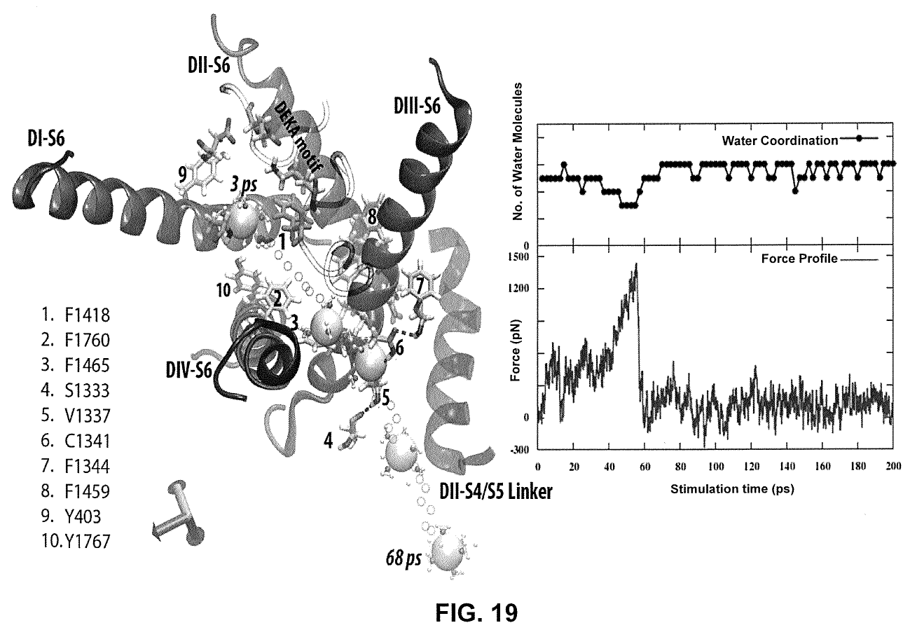

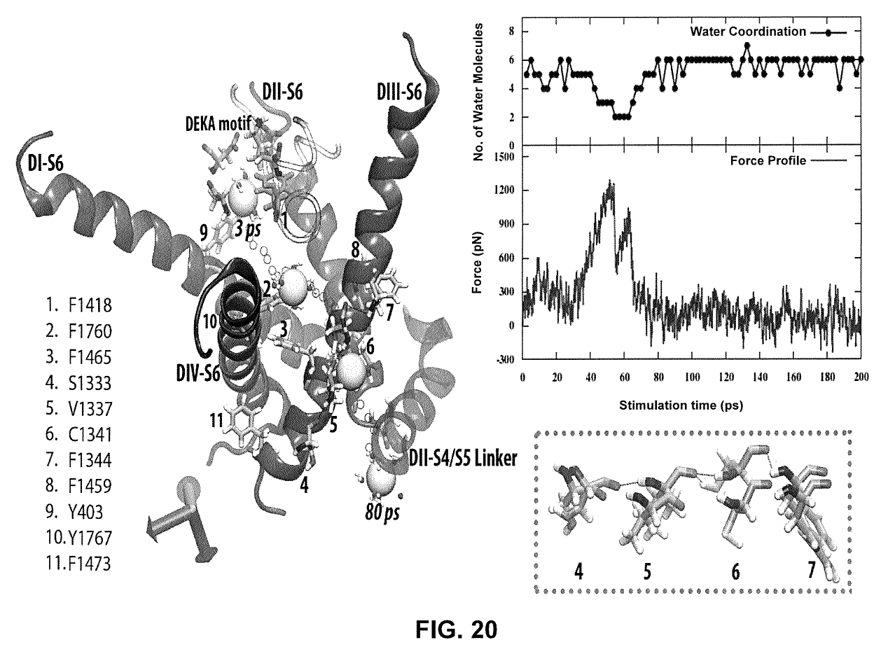

Since pathway 2 involved permeation along a very narrow cavity, the ion had to drop most of the water molecules from its solvation shell, which reduced the number to as low as two water molecules. Particularly, when the ion passed through the ruptured H-bond fence and traversed towards the S4/S5 linker (of DII), the ion was accompanied by only two water molecules. The ion regained its full solvation shell after it permeated in the bulk water. Similar mechanisms of ion permeation via pathway 2 were also observed in the SMD simulations based on the snap3 and snap7 structures, as illustrated in FIGS. 19 and 20. In these examples, the ion passage through the pathway 2 were influenced by aromatic residues, Phe1760 and Phe1465, a cascade of H-bonds formed by Ser1333-Val1337-Cys1341-Phe1344 interactions and the dynamics of S4/S5 helical linker of DII.

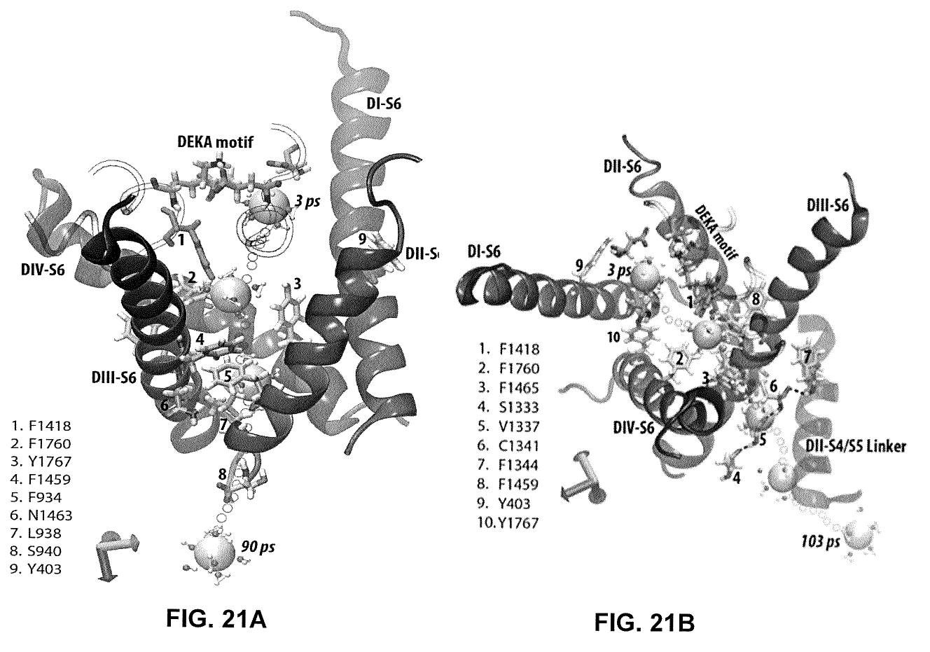

In comparison, the ion permeation based on the snap5 structure of the MD simulation in EXAMPLE 3 occurred via pathway 1 or pathway 2 during repeated simulations, as illustrated in FIGS. 21A and 21B. This result is likely caused by the orientation of Phe1418 residue. As illustrated in the FIGS. 21A and 21B, the aromatic side chain of Phe1418 is neither tilted towards pathway 1 nor on pathway 2. Instead Phe1418 is placed exactly in the center, leaving both, pathway 1 and pathway 2, sufficiently clear for ion permeation. Hence, the ion trapped in the central cavity of the snap5 structure permeated along both pathways during the SMD simulations with k=4 kcal/mol or 5 kcal/mol and v=0.45 Å/ps. The dissociation mechanisms, force profiles and water coordination for the snap 5 SMD trajectories via pathway 1 and pathway 2 were similar to those based on the SMD simulations based on the other snapshots. That is, the ion permeation through pathway 1 for the snap5 structure takes place by rupturing the stacking of Phe1459 and Phe934 and the H-bond interactions between Asn1463 and Leu938. In the case of pathway 2 for the snap5 structure, the ion breaks through the H-bond bridge formed by Ser1333-Val1337-Cys1341-Phe1344 interactions and the dynamics of S4/S5 helical linker of DII.

Permeating via pathway 2, however, is likely less probable under physiological conditions, since the solvation shell of the sodium ion drops significantly while permeating along this pathway. In addition, forces in force profiles for pathway 2 are higher than those for pathway 1, indicating that the energetic barriers of pathway 2 could potentially be larger than of pathway 1. Moreover, the ion permeation in pathway 2 changed its direction towards the Ser1333-Val1337-Cys1341-Phe1344 H-bonds only because the Phe1419 and Phe892 stacking did not clear the path for further movement of ion between the S6 helices of DIII and DIV. Thus, mutating one of these Phe residues could likely open up the cavity between the S6 helices of DIII and DIV for ion permeation.

6.5.3 Identification of Key Residues of Naᵥ1.5 Channel's Permeation Pathways

The SMD simulations identified a number of phenylalanine residues (Phe892, Phe934, Phe1418, Phe1459, Phe1465, Phe1760) to control the permeation of sodium ion through the S6 helices of the ion channel. In agreement with these findings, previous studies also reported that the small-molecule binding site in Naᵥ1.5 structure to be located around the Phe1760 residue (Chan et al., 2012 “Contribution of Local Anesthetic Binding Site Residues F1760 and Y1767 to Block of the Cardiac Na⁺ Channel, hNaᵥ1.5, by Ranolazine,” Biophys. J. 102: 324a; Beyder et al., 2012, “Ranolazine decreases mechanosensitivity of the voltage-gated sodium ion channel Na(v)1.5: a novel mechanism of drug action,” Circulation 125: 2698-2706; Wang et al., 2010, “Propranolol blocks cardiac and neuronal voltage-gated sodium channels,” Front. Pharmacol. 1: 144; Carboni et al., 2005, “Slow sodium channel inactivation and use-dependent block modulated by the same domain IV S6 residue,” J. Membr. Biol. 207: 107-117). Mutation of the Phe1760 residue has been shown to interfere with the binding and/or efficacy of several antiarrhythmic drugs, such as Ranolazine (Fredj et al., 2006, “Molecular basis of ranolazine block of LQT-3 mutant sodium channels: evidence for site of action,” Br. J. Pharmacol. 148: 16-24).

Other key residues based on the SMD simulations described herein include Phe366 (P1) and Trp374 (P2) of domain DI; Phe892 (P1) and Trp899 (P2) of DII; Phe1418 (P1) and Trp1421 (P2) of DIII; and Phe1705 (P1) and Trp1713 (P2) of DIV. In addition, sidechain conformational changes in Phe892 (DII), Phe1418 (DIII) and Trp1713 (DIV) were significant determinants of ion permeation passages via either the DII-DIII S6 helices or the DIII-DIV S6 helices as determined by the SMD simulations described herein. Furthermore, stacking arrangements of two oppositely facing Phe residues, Phe1459 (from DIII-S6 helix) and Phe934 (from DII-S6) and accompanying hydrogen bond interactions between Asn1463 (from DIII-S6) and Leu938 (from DII-S6) played an important role in the permeation pathways, indicating these residues to be key permeation residues. and accompanied by strong hydrogen bonds between Asn1774 and Asn1472 at the tip of DIII-DIV S6 helices.

Addition key residues identified in the SMD simulations described herein include: Ser1333, Val1337, Cys1341, Phe1344, Tyr403, Tyr416, Asn 932, Tyr1767, Phe 402, and Phe 389, wherein the cation-π interaction between Tyr416 (S6 of DI) and Asn932 (S6 of DII) guarded the passage between the S6 helices of DI and DII, and Tyr1767 (DIV), Phe402 and Phe389 (DI) severely blocked the path between DIV-DI S6 helices.

Atomistic RMSD-based clustering of the residues surrounding the key residues were performed in order to explore the conformational space of the small-molecule binding site of Naᵥ1.5 protein.

### 6.6 Example 6: Clustering Analysis and Protein Preparation for Docking Studies

Iterative clustering of the MD trajectory of EXAMPLE 3 was then performed to extract dominant conformations of hNaᵥ1.5 channel. The clustering procedure has been previously described (Barakat et al., 2010, “Ensemble-Based Virtual Screening Reveals Dual-Inhibitors for the P53-MDM2/MDMX Interactions,” J. Mol. Graph. & Model. 28, 555-568; Barakat et al., 2011, “Relaxed Complex Scheme Suggests Novel Inhibitors for the Lyase Activity Of DNA Polymerase Beta,” J. Mol. Graph. & Model. 29, 702-716).

The dominant binding site conformations of the hNaᵥ1.5 channel were selected by clustering the RMSDs of the 580 ns classical MD trajectory using the Davies-Bouldin index (DBI) and the elbow criterion defined by the ratio between Sum of Squares Regression (SSR) to the Total Sum of Squares (SST). This algorithm was used to group similar conformations in the 580 ns trajectory into clusters. The optimal number of clusters was estimated by observing the values of the Davies-Bouldin index (DBI) (see, e.g., Davies et al., 1979, “A Cluster Separation Measure,” IEEE Trans. Pattern Anal. Intelligence 1, 224) and the percentage of data explained by the data (SSR/SST) (see, e.g., Shao et al., 2007, “Clustering Molecular Dynamics Trajectories: 1. Characterizing the Performance of Different Clustering Algorithms,” J. Chem. Theory & Computation. 3, 231) for different cluster counts ranging from 5 to 600. At the optimal number of clusters, a plateau in the SSR/SST is expected to match a local minimum in the DBI (Shao et al., 2007). The optimal number is expected when a plateau in SSR/SST coincides with a local minimum for the DBI.

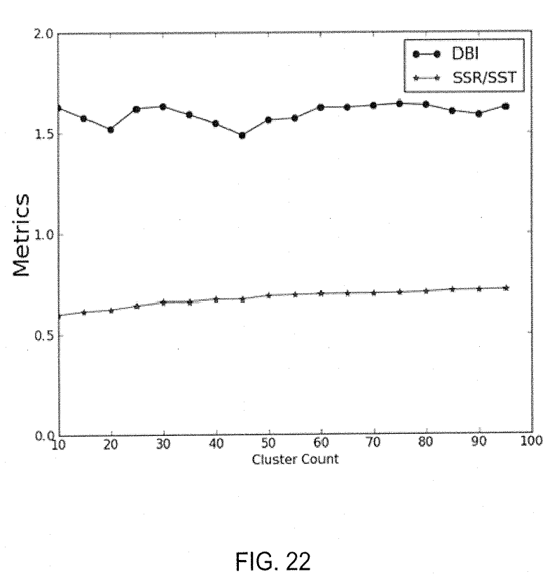

Convergence of the clustering was assessed by the elbow criterion of the DBI versus the SSR/SST plot and an ensemble of dominant Naᵥ1.5 protein conformations were obtained. As illustrated in FIG. 22, clustering converges at approximately 20 dominant protein conformations. The dominant conformations of the protein identified through cluster analysis were subsequently prepared and refined using the protein preparation wizard in Schrodinger software package. (Schrödinger LLC., Portland, USA; http://www.schrodinger.com). These dominant binding site conformations were used to test the capability of the Naᵥ1.5 model to efficiently rank the known Naᵥ1.5 blockers. All 20 dominant protein conformations are superimposed and given in FIG. 13.

### 6.7 Example 7: Docking Studies

Docking studies were performed next. The ability of the model to predict drug-mediated Naᵥ1.5 blockage has been tested against 35 ligands, which are known to block Naᵥ1.5 strongly (IC₅₀<1 μM, 24 compounds) or weakly (IC₅₀>25 μM, 8 compounds). Additionally, three marketed antiarrhythmic Naᵥ1.5 blockers, Ranolazine (IC₅₀ of 5.9 μM) (Antzelevitch et al., 2004, “Electrophysiological effects of ranolazine, a novel antianginal agent with antiarrhythmic properties,” Circulation 110: 904-910), Lidocaine (IC₅₀ of 10.8 μM) (Plouvier et al., 2007, “Synthesis and biological studies of novel 2-aminoalkylethers as potential antiarrhythmic agents for the conversion of atrial fibrillation,” J. Med. Chem. 50: 2818-2841), and Flecainide, have also been included. Flecainide has been shown to bind strongly to the open activated state of the channel (IC₅₀ 7 μM) and only very weakly to the closed/inactivated state (IC₅₀ 345 μM) (Ramos and O'Leary, 2004, “State-dependent trapping of flecainide in the cardiac sodium channel,” J. Physiol. 560: 37-49). The chemical structures of these 35 compounds are shown in Table 1.

All the ligands were prepared using the LigPrep module in Schrodinger drug discovery suite followed by ensemble-based docking against dominant hNaᵥ1.5 conformations obtained from RMSD clustering of EXAMPLE 6

Docking was performed using the Glide standard precision docking module (Glide SP) from Schrödinger (Schrödinger LLC., Portland, USA; http://www.schrodinger.com) and enhanced search parameters. A maximum of 20,000 poses were kept from the first phase of docking where the best 1000 poses were moved to refinement. The best 50 poses of these were subjected to 200 steps minimization. The best 500 scoring poses for each ligand were clustered using a 1 Å RMSD tolerance. A modified docking score based on the Ruvinsky algorithm were calculated for each ligand (Ruvinsky, 2007, “Role of binding entropy in the refinement of protein-ligand docking predictions: analysis based on the use of 11 scoring functions,”. J. Comp. Chem. 28: 1364-1372). This algorithm takes into account the number of poses within each cluster well and has been shown to be very successful in accounting for the entropy of the generated poses by different docking programs. The best seven poses for each ligand were selected for subsequent MD simulations and binding free energy calculations as described in EXAMPLE 6.

### 6.8 Example 8: Binding Free Energy Calculation and Rescoring of Top Hits

For each pose, the binding energies were computed using the popular MMGBSA module of AMBER to re-score the preliminary ranked docking hits (Kollman et al., 2000, “Calculating Structures and Free Energies of Complex Molecules: Combining Molecular Mechanics and Continuum Models,” Acc. Chem. Res. 3B, 889-897). This technique combines molecular mechanics with continuum solvation models. The total free energy is estimated as the sum of average molecular mechanical gas-phase energies (E_{MM}), solvation free energies (Gₛₒₗᵥ), and entropy contributions (−TSₛₒₗᵤₜₑ) of the binding reaction:

G=E_{MM}+Gₛₒₗᵥ−TSₛₒₗᵤₜₑ (3)

The molecular mechanical (E_{MM}) energy of each snapshot was calculated using the SANDER module of AMBER10 with all pair-wise interactions included using a dielectric constant (c) of 1.0. The solvation free energy (Gₛₒₗᵥ) was estimated as the sum of electrostatic solvation free energy, calculated by using the generalized Born (GB) model, and non-polar solvation free energy, calculated from the solvent-accessible surface area (SASA) algorithm. The solute entropy was approximated using the normal mode analysis. Applying the thermodynamic cycle for each protein-ligand complex, the binding free energy was calculated using the following equation:

ΔG_{calc}^{o}=ΔG_{gas}^{hNav1.5-ligand}+ΔGₛₒₗᵥ^{hNav1.5-ligand}−{ΔGₛₒₗᵥ^{ligand}+ΔGₛₒₗᵥ^{hNav1.5}}. (4)

Here, (ΔG_{gas}^{hNav1.5-ligand}) represents the free energy per mole for the non-covalent association of the ligand-protein complex in vacuum (gas phase) at a representative temperature, while (−ΔGₛₒₗᵥ) stands for the work required to transfer a molecule from its solution conformation to the same conformation in vacuum (assuming that the binding conformation of the ligand-protein complex is the same in solution and in vacuum).

The calculated binding energies, ΔG^{o}_{calc}, can be compared directly to the physiologically relevant concentrations. In this regard, the IC₅₀ (concentration at which 50% inhibition is observed) values measured from, for example, in vitro biological assays are converted to the observed free energy change of binding, ΔG_{obs} (cal mol⁻¹) using the equation:

ΔG^{o}_{obs}=RT lnKᵢ, (5)

where R is the gas constant, R=1.987 cal K⁻¹mol⁻¹, T is the absolute temperature, and Kᵢ is approximated to be the IC₅₀ measured for a particular test compound, i. Accordingly, the calculated binding energies in silico, ΔG^{o}_{calc}, are compared to the observed binding energy in vitro, ΔG_{obs} (e.g., from inhibition studies), and thus, also to the physiologically relevant concentrations (IC₅₀) for each of the combinations of compound and protein, for example, the hNaᵥ1.5 channel.

The calculated binding energy of a tested compound may also compared to that of a known control (a known hNaᵥ1.5 blocker from a standardized panel of drugs). The following equation is used:

$$Δ {G}_{1} - Δ {G}_{2} = RT ln ( \frac{{K}_{i 1}}{{K}_{i 2}} ) , \qquad ( 6 )$$

where Kᵢ₁ and Kᵢ₂ are the molar concentrations of the tested compound and the control, respectively.

The seven best-scoring poses for each ligand from EXAMPLE 7 were selected for carrying out short constrained MD simulations. In order to save the computational cost and to speed up the calculations, the lipid bilayers was not included in these simulations. This is acceptable as at this short time scale the Naᵥ1.5 channel likely preserve its overall conformation and only the orientation of the residues interacting with the bound molecule slightly changes to accommodate the bound ligand. To prepare these complexes, we followed a standard protein system preparation and setup using the AMBER software package (Salomon-Ferrer et al., 2013, “An overview of the Amber biomolecular simulation package,” Wiley Interdisciplinary Reviews: Computational Molecular Science 3: 198-210). Each protein-ligand complex was immersed in a 12 Å³ sized cubic box of TIP3P water molecules and the parameters for the amino acids and ligand molecules were assigned using the AMBER-FF99SB force field and the GAFF force field, respectively. Ligand charges were calculated and fitted to the atomic centers using the AM1-BCC method in antechamber. Each system was then subjected to four consecutive steps of constrained energy minimization, in which the constraints were gradually reduced in the following order: 100→50→5→zero kcal/mol.

In each system, the protein and ligand were constrained to their reference positions using a 5 kcal/mol of force and slowly heated to 300 K over 50,000 steps. The systems were then equilibrated for 20,000 steps, where the heavy atoms of the protein and ligand were constrained with a force constant of 1 kcal/mol in the first 10,000 steps and 0.1 kcal/mol in the last 10,000 steps. Final equilibrations were carried out for 25,000 steps while retaining the constraints only on the heavy atoms of protein, but with a much reduced force constant of 0.1 kcal/mol. Later, four 250 ps production simulations, thus a total of 1 ns simulation, were performed for each system, which has been shown to be an efficient approach in making reliable binding free energy predictions. (Su et al., 2015, “Comparison of radii sets, entropy, QM methods, and sampling on MM-PBSA, MM-GBSA, and QM/MM-GBSA ligand binding energies of F. tularensis enoyl-ACP reductase (FabI),”. J. Comp. Chem. 36: 1859-1873). All simulations were carried out using periodic boundary conditions and with an NPT ensemble. Short-range electrostatic interactions were truncated after 9 Å and long range electrostatic interactions were calculated using the Particle Mesh Ewald summation. An integration time step of 1 fs was used. For each system, the binding energies were computed using the popular MMGBSA module of AMBER over the 1 ns production simulation and using a 4 ps frame separation interval. (Miller et al., 2012, “MMPBSA.py: An Efficient Program for End-State Free Energy Calculations,” J. Chem. Theory Comp. 8: 3314-3321). The in vitro measured IC₅₀ values were then correlated against the calculated AMBER-MMGBSA binding energies to assess the success of the model to discriminate strong from weak Naᵥ1.5 blockers.

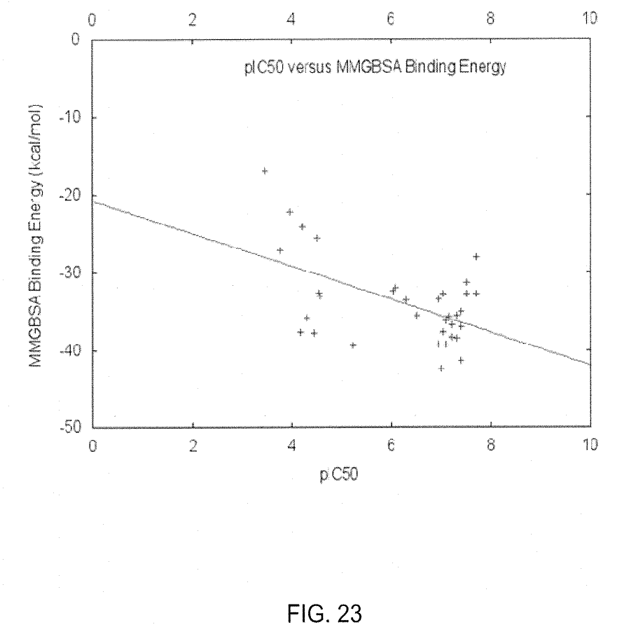

Table 3 lists the compound nos. of Table 1, the IC₅₀, the pIC₅₀ and the calculated AMBER-MMGBSA binding energies of the tested molecules. The Pearson correlation coefficients (rₚₑₐᵣₛₒₙ) between the IC₅₀ against the AMBER-MMGBSA binding energies showed a good correlation, rₚₑₐᵣₛₒₙ=0.7, of the measured IC₅₀ and the calculated binding energies. For example, Flecainide, which was previously shown to inhibit the closed state of Naᵥ1.5 very weakly (IC₅₀ 345 μM) (Ramos and O'Leary, 2004), indeed exhibited the highest binding energy (weakest affinity) among all the inhibitors under study (−16.79 kcal/mol). On the other hand, Ranolazine exhibited one of the lowest binding energies (strong affinity) among the tested inhibitors, with −39.35 kcal/mol. FIG. 23 illustrates a scatter plot of the pIC₅₀ values against the calculated binding energies, fitting data points according to the following equation: f(x)=ax+b, where a is given by −2.12 and b is given by −20.74. In general, the Naᵥ1.5 model displayed a good tendency to predict strong blockers correctly. For example, with the exception of CHEMBL2012259, none of the strong blockers (IC₅₀<1 μM) exhibited a binding energy above −30.00 kcal/mol. Calculating the AMBER-MMGBSA binding energies and rescoring the ligands based on these binding energies over the selected snapshots improved the ranking of the chosen compounds based on their corresponding IC₅₀ values, such that the experimentally observed activity trend is reproduced (Ranolazine>Dofetilide>Nadolol).

TABLE 3
Binding Energy Scores from Some Selected
Compounds Against hNaᵥ1.5.

| | | | AMBER- |
|---|---|---|---|
| | | | MMGBSA |
| | | | Binding |
| Compound | IC₅₀ | | Energy |
| No. | (nM) | pIC₅₀ | (kcal/mole) |
| 1 | 20 | 7.7 | −28.07 |
| 2 | 20 | 7.7 | −32.85 |
| 3 | 30 | 7.52 | −32.78 |
| 4 | 30 | 7.52 | −31.38 |
| 5 | 40 | 7.4 | −41.35 |
| 6 | 40 | 7.4 | −35.04 |
| 7 | 40 | 7.4 | −37.05 |
| 8 | 50 | 7.3 | −38.47 |
| 9 | 50 | 7.3 | −35.56 |
| 10 | 60 | 7.22 | −38.37 |
| 11 | 60 | 7.22 | −36.79 |
| 12 | 70 | 7.15 | −35.68 |
| 13 | 80 | 7.1 | −36.03 |
| 14 | 80 | 7.1 | −36.25 |
| 15 | 80 | 7.1 | −39.29 |
| 16 | 90 | 7.05 | −37.66 |
| 17 | 90 | 7.05 | −32.87 |
| 18 | 100 | 7.0 | −42.33 |
| 19 | 110 | 6.96 | −33.47 |
| 20 | 110 | 6.96 | −39.25 |
| 21 | 300 | 6.52 | −35.50 |
| 22 | 500 | 6.3 | −33.54 |
| 23 | 810 | 6.09 | −32.06 |
| 24 | 940 | 6.03 | −32.39 |
| 25 | 5900 | 5.23 | −39.35 |
| 26 | 27000 | 4.57 | −33.08 |
| 27 | 28000 | 4.55 | −32.65 |
| 28 | 31622.78 | 4.5 | −25.63 |
| 29 | 36000 | 4.44 | −37.85 |
| 30 | 50000 | 4.3 | −35.83 |
| 31 | 62000 | 4.21 | −24.18 |
| 32 | 69000 | 4.16 | −37.73 |
| 33 | 108000 | 3.97 | −22.24 |
| 34 | 174000 | 3.76 | −27.21 |
| 35 | 345000 | 3.46 | −16.79 |

### 6.9 Example 9: Classification of Channel Binding Mode

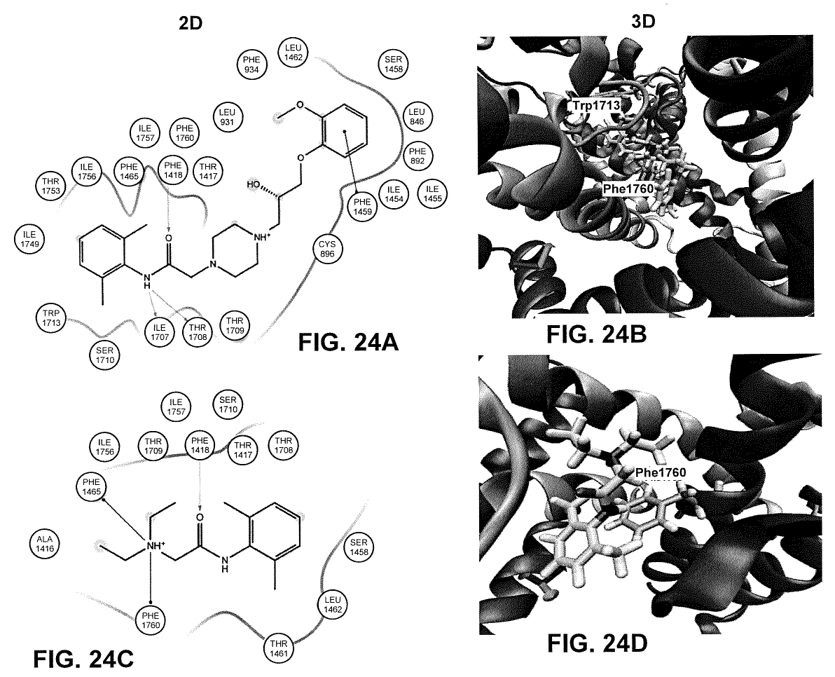

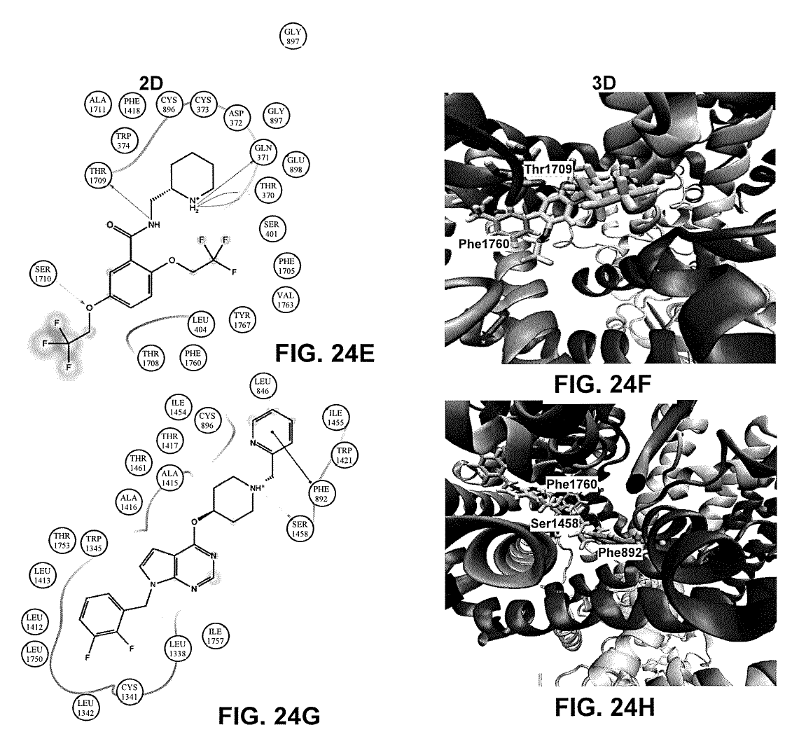

To gain more insights into the predicted binding modes of some of the tested compounds with the hNaᵥ1.5 model, the exact binding modes of the lowest energy complexes of Ranolazine, Lidocaine, Flecainide and CHEMBL2012299 (Compound No. 2), were analyzed. The first three compounds are known anti-arrhythmic drugs in the market and the last compound is presumably one of the strongest hNaᵥ1.5 blockers reported so far (IC₅₀=20 nM) (Chakka et al., 2012, “Discovery and hit-to-lead optimization of pyrrolopyrimidines as potent, state-dependent Na(v)1.7 antagonists,” Bioorg. Med. Chem. Lett. 22: 2052-2062). FIGS. 24A-24H illustrate the 2D and 3D ligand interaction diagrams of the four compounds, wherein Ranolazine, Lidocaine and Flecainide bind to the center of the channel, and CHEMBL2012299 binds slightly shifted to the fenestration sides of the hNaᵥ1.5 channel. VMD (Visual MD) (Humphrey et al., 1996, “Visual Molecular Dynamics,” J. Mol. Graphics, 14 (1), 33-38) was used to visually analyze the results of the MD trajectories of the these selected complexes of EXAMPLE 8.

A channel blocker binds within the cavity so that the passage of the sodium ions through the selection filter is blocked. On the other hand, a compound may bind to the channel in a way that it does not interfere with the sodium passage. With that in mind, and by visually inspecting the bound structures, one can classify the tested small molecules as “blockers,” e.g., compounds that blocked the hNaᵥ1.5 channel, or as “non-blockers,” e.g., compounds that did not block the hNaᵥ1.5 channel. FIGS. 24A-24H present examples of weak blockers (Ranolazine, Lidocaine, Flecainide) and a strong blocker (CHEMBL2012299) bound to the hNaᵥ1.5 model.

The benzamide aromatic head of Ranolazine was flanked between the two aromatic rings of Phe1760 and Trp1713 (FIGS. 24A and 24B). The benzamide nitrogen form strong H-bonds with the backbone carbonyl oxygens of Ile1707 and Thr1708 and the side chain hydroxyl of Ser1710. Consistent with the data, it was previously reported that Phe1760 mutation to Ala1760 significantly reduces Ranolzaine efficacy as a potent antiarrhythmic drug in vitro (Fredj et al., 2006, “Molecular basis of ranolazine block of LQT-3 mutant sodium channels: evidence for site of action,” Br. J. Pharmacol. 148: 16-24). This presumably occurred as a result of perturbing the cluster of lipophilic residues that surround the benzamide group of Ranolazine, such as Phe1418, Phe1465, Ile1756 and Ile1757 upon mutating the Phe1760 residue. It is important to note that the SMD simulations reported herein identified Phe1418 and Phe1465 to play important roles in the ion permeation from the central cavity into the intracellular solution. Furthermore, the predicted binding mode of Ranolzaine was located far from Tyr1767, excluding any direct interaction with this residue. As shown in FIGS. 24A and 24B, Ranolazine forms a direct, sandwich type π-π stacking interaction with Phe1760, and tilted T-shaped type π-π stacking interaction with Tyr1767. Again consistent with previously reported experimental data that showed that Tyr1767 does not interact with Ranolazine (Pless et al., 2011, “Molecular basis for class Ib anti-arrhythmic inhibition of cardiac sodium channels,” Nat. Commun. 2: 351). Data from the SMD simulations described herein further indicated that Trp1713 plays an important role as a target site for binding Ranolazine.

The second blocker, Lidocaine, exhibited a binding mode that is somehow different from Ranolazine. Lidocaine is presumably the most studied Naᵥ1.5 small molecule blocker (Pless et al., 2011; Hanck et al., 2009, “Using lidocaine and benzocaine to link sodium channel molecular conformations to state-dependent antiarrhythmic drug affinity,” Circ. Res. 105: 492-499). There is a debate in the literature regarding which ionization state of Lidocaine interacts with Naᵥ1.5 and whether the exact action of Lidocaine is due to a state dependent or independent block of Naᵥ1.5. Hanck et al. discussed the hypothesis of the Lidocaine's ability to exert the blocking action on Naᵥ1.5 through interaction with both an open and closed state of the channel, and the possibility of whether the Naᵥ1.5 blocking activity of Lidocaine can be related to its charged state, being a weak base (pKa=7.96). Hanck et al. concluded that Lidocaine can interact in its charged (+1) state with an open state of the channel, whereas it interacts with the closed state of the channel in a neutral form and that both types of interactions are important to achieve an optimum blocking effect. However, Pless et. al. more recently proven, through a combination of experiments and ab initio electrostatic potential calculations, that charged (+1) Lidocaine interacts with the closed state of Naᵥ1.5 through a strong cation-pi stacking interaction with Phe1760. Based on the SMD simulations described herein, the most favorable binding pose of Lidocaine involved a charged Lidocaine binding strongly through a cation-pi stacking interaction with Phe1760 while also interacting with Phe1465 and Phe1418 (FIGS. 24C and 24D)

The third blocker, Flecainide, exhibited the weakest binding energy among the whole list of inhibitors under study (binding energy=−16.79 kcal/mol) and also the lowest inhibition against hNaᵥ1.5 (I_{C50}=345 μM for the closed state). Flecainide is known to bind the open state of hNaᵥ1.5 (Ramos and O'Leary, 2004), which explains the calculated low binding energy against the closed hNaᵥ1.5 model. Unlike Lidocaine that interacted directly and strongly with Phe1760, Flecainide did not show such interaction, even though both drugs are representatives of class 1b antiarrhythmic drugs. The data of the SMD simulation is in accordance with Pless et. al (2011) that showed that Flecainide does not interact directly with Phe1760. Instead, as illustrated in FIGS. 24E and 24F, the benzamide nitrogen of Flecainide is H-bonded to the backbone carbonyl oxygen of Thr1709 and the di-triflouroethoxy aromatic ring forms a lipophilic interaction with Phe1760, Leu404, Tyr1767, Val1763 and Phe1705. None of these interactions, however, was strong enough to provide a stable interaction with this state of the hNaᵥ1.5 channel.

In contrast to Ranolazine, Lidocaine and Flecainide that bind directly in the vicinity of Phe1760, CHEMBL2012299 was slightly shifted toward the lipophilic fenestration sites of the hNaᵥ1.5 channel. These lipophilic fenestration sites are believed to be the route of entry of the small molecule VGSCs blockers to reach their designated binding site (Martin and Corry, 2014, “Locating the route of entry and binding sites of benzocaine and phenytoin in a bacterial voltage gated sodium channel,” PLoS Comput. Biol. 10: e1003688; Boiteux et al., 2014, “Local anesthetic and antiepileptic drug access and binding to a bacterial voltage-gated sodium channel,” Proc. Natl. Acad. Sci. USA 111: 13057-13062). As illustrated in the FIGS. 24G and 24H, the diflouro-phenyl ring of CHEMBL2012299 engaged with a cluster of lipophilic residues, such as Leu1338, Leu1342, Leu1750, Leu142 and Leu1413. The hydrophilic charged head at the other side of the molecule engaged with Ser145 forming a strong charge-assisted H-bond. The terminal pyridine ring formed a strong pi-pi stacking interaction with Phe892.

### 6.10 Example 10: Computations for Compound Selection

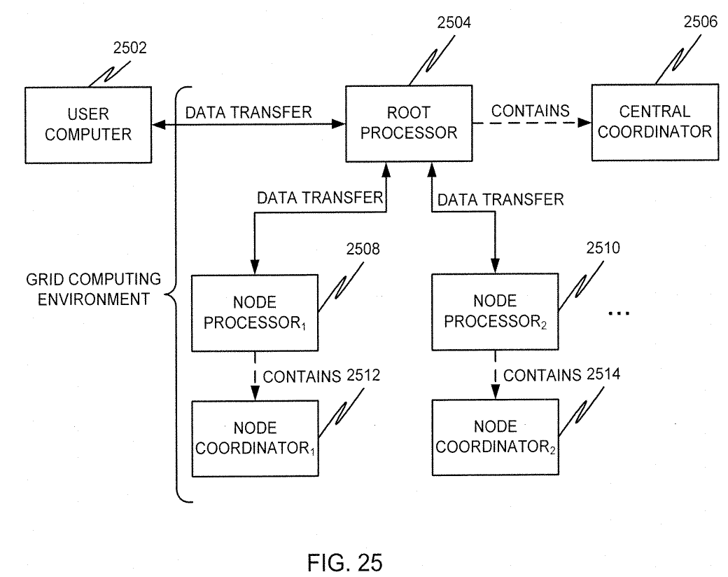

FIG. 25 depicts a grid computing environment for selecting a compound with reduced risk of cardiotoxicity. As shown in FIG. 25, user computers 2502 can interact with the grid computing environment 2506 through a number of ways, such as over one or more networks 2504. The grid computing environment 2506 can assist users to select a compound with reduced risk of cardiotoxicity.

One or more data stores 2508 can store the data to be analyzed by the grid computing environment 2506 as well as any intermediate or final data generated by the grid computing environment. However in certain embodiments, the configuration of the grid computing environment 2506 allows its operations to be performed such that intermediate and final data results can be stored solely in volatile memory (e.g., RAM), without a requirement that intermediate or final data results be stored to non-volatile types of memory (e.g., disk).

This can be useful in certain situations, such as when the grid computing environment 2506 receives ad hoc queries from a user and when responses, which are generated by processing large amounts of data, need to be generated on-the-fly. In this non-limiting situation, the grid computing environment 2506 is configured to retain the processed information within the grid memory so that responses can be generated for the user at different levels of detail as well as allow a user to interactively query against this information.

For example, the grid computing environment 2506 receives structural information describing the structure of the ion channel protein, and performs a molecular dynamics simulation of the protein structure. Then, the grid computing environment 2506 uses a clustering algorithm to identify dominant conformations of the protein structure from the molecular dynamics simulation, and select the dominant conformations of the protein structure identified from the clustering algorithm. In addition, the grid computing environment 2506 receives structural information describing conformers of one or more compounds, and uses a docking algorithm to dock the conformers of the one or more compounds to the dominant conformations. The grid computing environment 2506 further identifies a plurality of preferred binding conformations for each of the combinations of protein and compound, and optimizes the preferred binding conformations using molecular dynamics simulations so as to determine whether the compound blocks the ion channel of the protein in the preferred binding conformations.

Specifically, in response to user inquires about cardiotoxicity of a compound, the grid computing environment 2506, without an OLAP or relational database environment being required, aggregates protein structural information and compound structural information from the data stores 2508. Then the grid computing environment 2506 uses the received protein structural information to perform molecular dynamics simulations for determining configurations of target protein flexibility (e.g., over a simulation length of greater than 50 ns). The molecular dynamics simulations involve the grid computing environment 2506 determining forces acting on an atom based upon an empirical force field that approximates intramolecular forces, where numerical integration is performed to update positions and velocities of atoms. The grid computing environment 2506 clusters molecular dynamic trajectories formed based upon the updated positions and velocities of the atoms into dominant conformations of the protein, and executes a docking algorithm that uses the compound's structural information in order to dock the compound's conformers to the dominant conformations of the protein. Based on information related to the docked compound's conformers, the grid computing environment 2506 identifies a plurality of preferred binding conformations for each of the combinations of protein and compound. If the compound does not block the ion channel of the protein in the preferred binding conformations, the grid computing environment 2506 predicts the compound has reduced risk of cardiotoxicity. Otherwise, the grid computing environment 2506 predicts the compound is cardiotoxic, and redesigns the compound in order to reduce risk of cadiotoxicity.

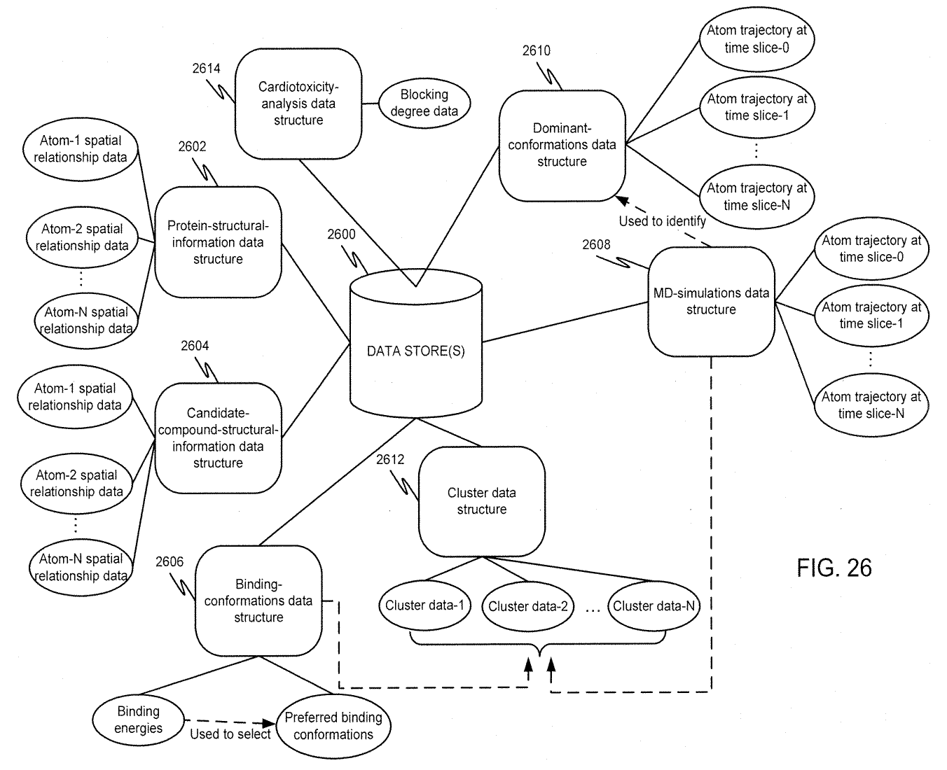

FIG. 26 illustrates hardware and software components for the grid computing environment 2506. As shown in FIG. 26, the grid computing environment 2506 includes a central coordinator software component 2606 which operates on a root data processor 2604. The central coordinator 2606 of the grid computing environment 2506 communicates with a user computer 2602 and with node coordinator software components (2612, 2614) which execute on their own separate data processors (2608, 2610) contained within the grid computing environment 1306.

As an example of an implementation environment, the grid computing environment 2506 can comprise a number of blade servers, and a central coordinator 2606 and the node coordinators (2612, 2614) are associated with their own blade server. In other words, a central coordinator 2606 and the node coordinators (2612, 2626) execute on their own respective blade server. In some embodiments, each blade server contains multiple cores and a thread is associated with and executes on a core belonging to a node processor (e.g., node processor 2608). A network connects each blade server together.

The central coordinator 2606 comprises a node on the grid. For example, there might be 100 nodes, with only 50 nodes specified to be run as node coordinators. The grid computing environment 2506 will run the central coordinator 2606 as a 51st node, and selects the central coordinator node randomly from within the grid. Accordingly, the central coordinator 2606 has the same hardware configuration as a node coordinator.

The central coordinator 2606 may receive information and provide information to a user regarding queries that the user has submitted to the grid. The central coordinator 2606 is also responsible for communicating with the 50 node coordinator nodes, such as by sending those instructions on what to do as well as receiving and processing information from the node coordinators. In one implementation, the central coordinator 2606 is the central point of contact for the client with respect to the grid, and a user never directly communicates with any of the node coordinators.

With respect to data transfers involving the central coordinator 2606, the central coordinator 2606 communicates with the client (or another source) to obtain the input data to be processed. The central coordinator 2606 divides up the input data and sends the correct portion of the input data for routing to the node coordinators. The central coordinator 2606 also may generate random numbers for use by the node coordinators in simulation operations as well as aggregate any processing results from the node coordinators. The central coordinator 2606 manages the node coordinators, and each node coordinator manages the threads which execute on their respective machines.

A node coordinator allocates memory for the threads with which it is associated. Associated threads are those that are in the same physical blade server as the node coordinator. However, it should be understood that other configurations could be used, such as multiple node coordinators being in the same blade server to manage different threads which operate on the server. Similar to a node coordinator managing and controlling operations within a blade server, the central coordinator 2606 manages and controls operations within a chassis.

In certain embodiments, a node processor includes shared memory for use for a node coordinator and its threads. The grid computing environment 2506 is structured to conduct its operations (e.g., matrix operations, etc.) such that as many data transfers as possible occur within a blade server (i.e., between threads via shared memory on their node) versus performing data transfers between threads which operate on different blades. Such data transfers via shared memory are more efficient than a data transfer involving a connection with another blade server.

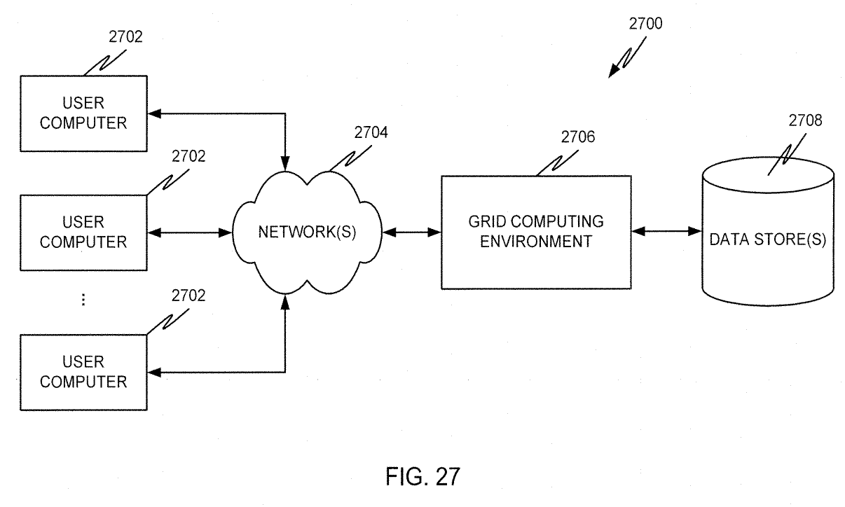

FIG. 27 depicts example schematics of data structures utilized by a compound-selection system. Multiple data structures are stored in a data store 2700, including a protein-structural-information data structure 2702, a candidate-compound-structural-information data structure 2704, a binding-conformations data structure 2706, a molecular-dynamics-simulations data structure 2708, a dominant-conformations data structure 2710, a cluster data structure 2712, and a cardiotoxicity-analysis data structure 2714. These interrelated data structures can be part of the central coordinator 2606 by aggregating data from individual nodes. However, portions of these data structures can be distributed as needed, so that the individual nodes can store the process data. The data store 2700 can be different types of storage devices and programming constructs (e.g., RAM, ROM, Flash memory, flat files, databases, programming data structures, programming variables, IF-THEN (or similar type) statement constructs, etc.). For example, the data store 2700 can be a single relational database or can be databases residing on a server in a distributed network.

Specifically, the protein-structural-information data structure 2702 is configured to store data related to the structure of the potassium ion channel protein, for example, special relationship data between different atoms. The data related to the structure of the potassium ion channel protein may be obtained from a homology model, an NMR solution structure, an X-ray crystal structure, a molecular model, etc. Molecular dynamics simulations can be performed on data stored in the protein-structural-information data structure 2702. For example, the molecular dynamics simulations involve solving the equation of motion according to the laws of physics, e.g., the chemical bonds within proteins being allowed to flex, rotate, bend, or vibrate. Information about the time dependence and magnitude of fluctuations in both positions and velocities of the given molecule/atoms is obtained from the molecular dynamics simulations. For example, data related to coordinates and velocities of molecules/atoms at equal time intervals or sampling intervals are obtained from the molecular dynamics simulations. Atomistic trajectory data (e.g., at different time slices) are formed based on the positions and velocities of molecules/atoms resulted from the molecular dynamics simulations and stored in the molecular-dynamics-simulations data structure 2708. The molecular dynamics simulations can be of any duration. In certain embodiments, the duration of the molecular dynamics simulation is greater than 50 ns, for example, preferably greater than 200 ns.

Data stored in the molecular-dynamics-simulations data structure 2708 are processed using a clustering algorithm, and associated cluster population data are stored in the cluster data structure 2712. Dominant conformations of the potassium ion channel protein are identified based at least in part on the data stored in the molecular-dynamics-simulations data structure 2708 and the associated cluster population data stored in the cluster data structure 2712. Atomistic trajectory data (e.g., at different time slices) related to the identified dominant conformations are stored in the dominant-conformations data structure 2710.

Data stored in the candidate-compound-structure-information data structure 2704 are processed together with data related to the dominant conformations of the potassium ion channel protein stored in the dominant-conformations data structure 2710. The conformers of the one or more compounds are docked to the dominant conformations of the structure of the potassium ion channel protein using a docking algorithm (e.g., DOCK, AutoDock, Glide-SP, etc.), so that data related to various combinations of potassium ion channel protein and compound is determined and stored in the binding-conformations data structure 2706. For example, the compound is an antiviral agent (e.g., hepatitis C inhibitor). As an example, the binding-conformations data structure includes data related to binding energies. 2D information of the compound may be translated into a 3D representative structure to be stored in the candidate-compound-structure-information data structure 2704 for docking. Data stored in the binding-conformations data structure 2706 are processed using a clustering algorithm, and associated cluster population data are stored in the cluster data structure 2712. One or more preferred binding conformations are identified based at least in part on the data stored in the binding-conformations data structure 2706 and the associated cluster population data stored in the cluster data structure 2712. For example, the preferred binding conformations include those with a largest cluster population and a lowest binding energy.

The identified preferred binding conformations are optimized using a scalable molecular dynamics simulations (e.g., through a NAMD software, etc.). In certain embodiments, binding energies are calculated (e.g., using salvation models, etc.) for each of the combinations of protein and compound (receptor and ligand) in the corresponding optimized preferred binding conformation(s). The calculated binding energies are output as the predicted binding energies for each of the combinations of protein and compound.

The cardiotoxicity-analysis data structure 2714 includes data related to a blocking degree of one or more compounds, e.g., in the preferred binding conformations. For example, the data stored in the cardiotoxicity-analysis data structure 2714 includes identification of blocking sites and non-blocking sites. The data stored in the cardiotoxicity-analysis data structure 2714 indicates a potential cardiac hazard when (i) a pocket within the hNaᵥ1.5 channel is classified as a blocking site and (ii) a ligand fits within the pocket and is within a predetermined binding affinity level. The data stored in the cardiotoxicity-analysis data structure 2714 does not indicate a potential cardiac hazard when a ligand binds to a pocket within the hNaᵥ1.5 channel that is classified as a non-blocking site. In some embodiments, if the compound does not block the ion channel (e.g., the blocking degree being lower than a threshold) in the preferred binding conformation(s), the compound is predicted to have reduced risk of cardiotoxicity, and the compound can be selected. In other embodiments, if the compound blocks the ion channel (e.g., the blocking degree being higher than the threshold) in the preferred binding conformation(s), the compound is predicted to be cardiotoxic. A molecular modeling algorithm can be used to chemically modify or redesign the compound so as to reduce the risk of cardiotoxicity (e.g., to reduce the blocking degree).

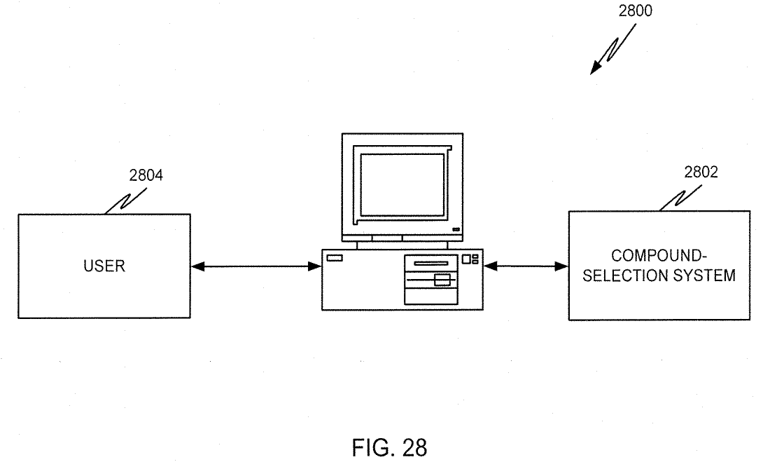

A system can be configured such that a compound-selection system 2802 can be provided on a stand-alone computer for access by a user 2804, such as shown at 2800 in FIG. 28.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform the methods and operations described herein. Other implementations may also be used, however, such as firmware or even appropriately designed hardware configured to carry out the methods and systems described herein.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The systems and methods may be provided on many different types of computer-readable media including computer storage mechanisms (e.g., CD-ROM, diskette, RAM, flash memory, computer's hard drive, etc.) that contain instructions (e.g., software) for use in execution by a processor to perform the methods' operations and implement the systems described herein.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope or of what may be claimed, but rather as descriptions of features specific to particular embodiments. Certain features that are described in this specification in the context or separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments have been described. Other embodiments are within the scope of the following claims. For example, the actions recited in the claims can be performed in a different order and still achieve desirable results.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Finally, as used in the description herein and throughout the claims that follow, the meanings of “and” and “or” include both the conjunctive and disjunctive and can be used interchangeably unless the context expressly dictates otherwise; the phrase “exclusive or” can be used to indicate situation where only the disjunctive meaning can apply.

While various illustrative embodiments are described above, it will be apparent to one skilled in the art that various changes and modifications may be made therein without departing from the invention. The appended claims are intended to cover all such changes and modifications.

All publications, patent publications and patent applications cited in this specification are herein incorporated by reference as if each individual publication or patent application were specifically and individually indicated to be incorporated by reference. Although the foregoing has been described in some detail by way of illustration and example for purposes of clarity of understanding, it will be readily apparent to those of ordinary skill in the art in light of the teachings of the specification that certain changes and modifications may be made thereto without departing from the spirit or scope of the appended claims.

Lengthy table referenced here
US20180068053A1-20180308-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20180068053A1). An electronic copy of the table will also be available from the USPTO upon request and payment of the fee set forth in 37 CFR 1.19(b)(3).

1. A method for selecting a compound with reduced risk of cardiotoxicity, comprising the steps of:
a) building a plurality of homology models of a closed state of a cardiac ion channel protein from structural information of the cardiac ion channel protein;
b) performing a molecular dynamics (MD) simulation of the plurality of homology models;
c) selecting one or more structures from the MD simulation;
d) performing a steered molecular dynamics (SMD) simulation of the one or more selected structures and an ion to identify one or more key residues of the cardiac ion channel protein's ion permeation pathways;
e) using a clustering algorithm to identify dominant conformations of the selected structure around the identified key residues of step d);
f) selecting the dominant conformations identified from the clustering algorithm;
g) providing structural information describing conformers of the compound;
h) using a docking algorithm to dock the conformers of the compound of step f) to the dominant conformations of step e);
i) identifying a plurality of preferred binding conformations for each of the combinations of the selected structure and the compound; and
j) calculating a binding energy of the compound using constrained MD simulations of the preferred binding conformations of step i);
wherein if the calculated binding energy of the compound in the preferred binding conformations is equal or less than the binding energy of a known blocker of the cardiac ion channel protein, the compound is predicted to be cardiotoxic; or
wherein if the calculated binding energy of the compound in the preferred binding conformations exceeds the binding energy of the known blocker of the cardiac ion channel protein, the compound is predicted to have reduced risk of cardiotoxicity; and
wherein based on the prediction that the compound has reduced risk of cardiotoxicity, the compound is selected;
wherein said steps a) through j) are executed on one or more processors.

2.-5. (canceled)

6. The method of claim 1, wherein the structural information of step a) is an X-ray crystal structure, an NMR solution structure, or a model determined using a protein structure prediction algorithm.

7.-13. (canceled)

14. The method of claim 1, wherein the cardiac ion channel protein is a sodium ion channel protein, and the sodium ion channel protein is hNaᵥ1.5.

15.-28. (canceled)

29. The method of claim 1, further comprising the step of providing an IC₅₀ values for each of the combinations of the cardiac, ion channel protein and the compound using the calculated binding energy of the compound and a function correlating IC₅₀ values and binding energies of compounds known to bind to the cardiac ion channel protein.

30. The method of claim 1, wherein if the compound is predicted to be cardiotoxic, the method further comprises the step of using a molecular modeling algorithm to chemically modify the compound such that the calculated binding energy of the modified compound in the preferred binding conformations exceeds the binding energy of the known blocker of the cardiac ion channel protein.

31. The method of claim 30, further comprising repeating steps a) through j) for the modified compound.

32. The method of claim 31, further comprising testing the cardiotoxicity of the compound or modified compound in an in vitro biological assay.

33. The method of claim 32, wherein the in vitro biological assay comprises high throughput screening of cardiac sodium ion channel and transporter activities.

34. The method of claim 32, wherein the in vitro biological assay is a hNaᵥ1.5 channel inhibition assay.

35. The method of claim 32, wherein the in vitro biological assay comprises electrophysiology measurements in single cells, whereas the electrophysiology measurements comprise patch clamp measurements.

36. The method of claim 14, wherein the one or more key residues are selected from the group consisting of Phe892, Phe934, Phe1418, Phe1459, Phe1465, and Phe1760.

37.-38. (canceled)

39. The method of claim 14, wherein the one or more key residues are selected from the group consisting of Phe366, Trp374, Phe892, Trp899, Phe1418, Trp1421, Phe1705, and Trp1713.

40. (canceled)

41. The method of claim 14, wherein the one or more key residues are selected from the group consisting of Phe389, Phe402, Tyr403, Tyr416, Asn932, Ser1333, Val1337, Cys1341, Phe1344, and Tyr1767.

42. The method of claim 14, wherein the one or more key residues are Phe366, Trp374, Phe389, Phe402, Tyr403, Tyr416, Phe892, Trp899, Asn932, Phe934, Leu938, Ser1333, Val1337, Cys1341, Phe1344, Phe1418, Trp1421, Phe1459, Asn1463, Phe1465, Asn1472, Phe1705, Trp1713, Tyr1767, or Asn1774.

43. (canceled)

44. The method of claim 1, wherein the one or more selected structures of step c) are selected from structures among a trajectory of the MD simulation.

45. The method of claim 1, wherein the building of a plurality of homology models of step a) comprises using the coordinates of Table A describing a structure of a closed state of a cardiac ion channel protein.

46. A computer-implemented system for selecting a compound with reduced risk of cardiotoxicity, the system comprising:
one or more data processors;
a computer-readable storage medium encoded with instructions for commanding the one or more data processors to execute operations performing the method of claim 1.

47. A non-transitory computer-readable storage medium storing a compound-selection program, the compound-selection program comprising instructions, which when executed by a processor of a data processing system, cause the data processing system to perform the method of claim 1.

48. A processor-implemented system for designing a compound in order to reduce risk of cardiotoxicity, comprising:
one or more computer-readable mediums for storing protein structural information representative of a closed state of a cardiac ion channel protein and for storing compound structural information describing conformers of the compound;
a grid computing system comprising a plurality of processor-implemented compute nodes and a processor-implemented central coordinator, said grid computing system receiving the stored protein structural information and the stored compound structural information from the one or more computer-readable mediums;
said grid computing system using the received protein structural information to build a plurality of homology models of the closed state of the cardiac ion channel protein, to perform molecular dynamics simulations for determining configurations of target protein flexibility over a simulation length of greater than 100 ns, and to perform steered molecular dynamics simulations for identifying one or more key residues of the cardiac ion channel protein's ion permeation pathways;
wherein the molecular dynamics and the steered molecular dynamics simulations involve each of the compute nodes determining forces acting on an atom based upon an empirical force field that approximates intramolecular forces; wherein numerical integration is performed to update positions and velocities of atoms;
wherein the central coordinator forms trajectories of the molecular dynamics or steered molecular dynamics simulations based upon the updated positions and velocities of the atoms as determined by each of the compute nodes;
said grid computing system configured to:
cluster the molecular dynamic trajectories into one or more dominant conformations of the cardiac ion channel protein around the identified key residues;
execute a docking algorithm that uses the compound's structural information in order to dock the compound's conformers to the one or more dominant conformations of the protein;
identify a plurality of preferred binding conformations for each of the combinations of protein and compound based on information related to the docked compound's conformers;
a data structure stored in memory which includes information about the one or more of the identified plurality of preferred binding conformations of the closed state of the cardiac ion channel protein;
whereby, based on a binding energy calculated from the information about the one or more of the identified plurality of preferred binding conformations, the compound is redesigned in order to reduce risk of cardiotoxicity.

49. A method for selecting a compound with reduced risk of cardiotoxicity, comprising the steps of:
a) using the coordinates of Table A describing a structure of a closed state of a cardiac ion channel protein;
b) performing a molecular dynamics (MD) simulation of the structure;
c) selecting one or more structure from the MD simulation;
d) performing a steered molecular dynamics (SMD) simulation of the one or more selected structures and an ion to identify one or more key residues of the cardiac ion channel protein's ion permeation pathways;
e) using a clustering algorithm to identify dominant conformations of the selected structure around the identified key residues of step d);
f) selecting the dominant conformations identified from the clustering algorithm;
g) providing structural information describing conformers of one or more compounds;
h) using a docking algorithm to dock the conformers of the one or more compounds of step f) to the dominant conformations of step e);
i) identifying a plurality of preferred binding conformations for each of the combinations of the selected structure and compound; and
j) calculating a binding energy of the compound using MD simulations of the preferred binding conformations of step i);
wherein if the calculated binding energy of the compound in the preferred binding conformations is equal or less than the binding energy of a known blocker of the cardiac ion channel protein, the compound is predicted to be cardiotoxic; or
wherein if the calculated binding energy of the compound in the preferred binding conformations exceeds the binding energy of the known blocker of the cardiac ion channel protein, the compound is predicted to have reduced risk of cardiotoxicity; and
wherein based on the prediction that the compound has reduced risk of cardiotoxicity, the compound is selected;
wherein said steps a) through j) are executed on one or more processors.

50. (canceled)
